(19) United States
(12) Patent Application Publication
(10) Pub. No.: US 2023/0339927 A1
(43) Pub. Date: Oct. 26, 2023

(54) ATR INHIBITORS AND USES THEREOF

(71) Applicant: ANTENGENE DISCOVERY LIMITED, Shanghai (CN)

(72) Inventors: Bo SHAN, Shanghai (CN); Bing HOU, Shanghai (CN); Hui YUWEN, Shanghai (CN); Zhongyang SHI, Shanghai (CN); Peng CHEN, Shanghai (CN); Jay MEI, Shanghai (CN)

(21) Appl. No.: 18/014,178

(22) PCT Filed: Jul. 2, 2021
(86) PCT No.: PCT/CN2021/104232

§ 371 Date: Jan. 3, 2023

(30) Foreign Application Priority Data
Jul. 3, 2020 (WO) PCT/CN2020/100088
Aug. 21, 2020 (WO) PCT/CN2020/110396
Dec. 9, 2020 (WO) PCT/CN2020/134732
Dec. 11, 2020 (WO) PCT/CN2020/135604

(51) Int. Cl. C07D 413/14

(52) U.S. Cl. CPC C07D 413/14

(57) ABSTRACT

The present disclosure relates to novel compounds useful as inhibitors of ATR kinase, as well as pharmaceutical compositions comprising these compounds and methods of treatment by administration of these compounds or the pharmaceutical compositions.

### FIELD OF THE DISCLOSURE

The present disclosure generally relates to novel compounds useful as ATR inhibitors, as well as pharmaceutical compositions comprising these compounds and methods of treatment by administration of these compounds or the pharmaceutical compositions.

### BACKGROUND OF THE DISCLOSURE

ATR (also known as FRAP-Related Protein 1; FRP1, MEC1, SCKL, SECKL1) protein kinase is a member of the PI3-Kinase like kinase (PIKK) family of proteins involved in repair and maintenance of the genome and its stability. It is essential to the viability of replicating cells and is activated during S-phase to regulate firing of replication origins and to repair damaged replication forks. Therefore, ATR inhibitors have the potential to be an efficient way in cancer treatment.

While progress has been made for ATR inhibitors, there is still a strong need in the art to develop improved pharmaceutics having inhibitory activity against ATR.

### SUMMARY OF THE DISCLOSURE

The present disclosure provides compounds, including stereoisomers, pharmaceutically acceptable salts, tautomers and prodrugs thereof, which are capable of inhibiting ATR protein kinase. Methods for use of such compounds for treatment of various diseases or conditions, such as cancer, are also provided.

In one aspect, the present disclosure provides a compound having Formula (I):

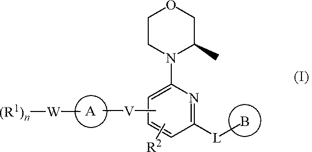

or a pharmaceutically acceptable salt thereof, wherein
Ring A is absent, 3- to 6-membered cycloalkyl, 5- to 6-membered heterocyclyl or 5- to 6-membered heteroaryl;
V is a direct bond, carbonyl or alkyl optionally substituted with one or more R^{c};
W and L are each independently a direct bond, —O—, —S—, or —N(R^{a})—;
R¹ is alkyl, cyano, —S(O)₂CH₃, or —S(O)(NH)CH₃;
R² is hydrogen, halogen or alkyl optionally substituted with one or more R^{b};
Ring B is

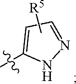

R⁵ is selected from the group consisting of hydrogen, halogen, hydroxyl, cyano, alkyl, alkenyl, alkynyl, heteroalkyl, heteroalkenyl, heteroalkynyl and haloalkyl;
R^{a} is hydrogen or alkyl;
R^{b} is hydroxyl or halogen;
R^{c} is hydroxyl, halogen or alkyl;
n is 0, 1, 2, or 3.

In some embodiments, the present disclosure provides compound having a Formula (II) or Formula (III):

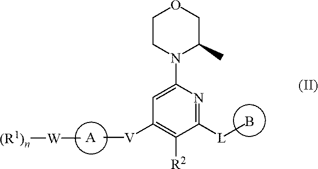

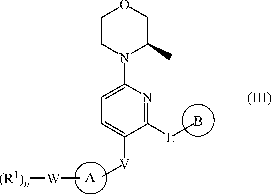

In some embodiments, the present disclosure provides compound having a formula selected from the group consisting of:

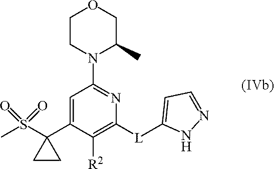

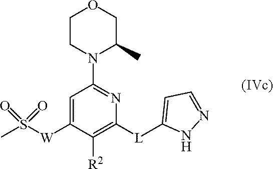

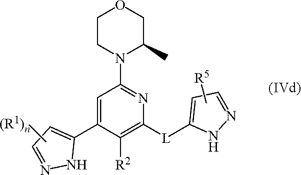

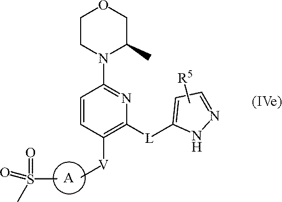

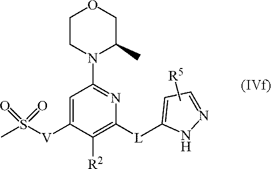

and

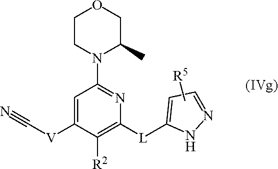

wherein
U is O or NH;
V is a direct bond, carbonyl or alkyl optionally substituted with one or more R^{c};
W and L are each independently -N(R^{a})-;
R¹ is alkyl;
R² is hydrogen, halogen or alkyl substituted with one or more R^{b};
R⁵ is hydrogen or alkyl;
R^{a} is hydrogen or alkyl;
R^{b} is hydroxyl or halogen; and
R^{c} is hydroxyl, halogen or alkyl.

In some embodiments, the present disclosure provides compound having Formula (V):

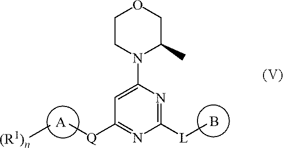

or a pharmaceutically acceptable salt thereof, wherein
Ring A is absent, 3- to 6-membered cycloalkyl, 5- to 6-membered heterocyclyl or 5- to 6-membered heteroaryl;
Q is a direct bond or alkyl optionally substituted with one or more R^{d};
L is —O—, —S—, or —N(R^{a})—;
Ring B is

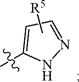

R^{a} is hydrogen or alkyl;
R^{d} is hydroxyl, halogen or alkyl;
R¹ is selected from the group consisting of cyano, hydroxyl, halogen, —S(O)₂CH₃, and —S(O)(NH)CH₃;
R⁵ is selected from the group consisting of hydrogen, halogen, hydroxyl, cyano, alkyl, alkenyl, alkynyl, heteroalkyl, heteroalkenyl, heteroalkynyl and haloalkyl;
n is 0, 1, 2, or 3.

In another aspect, the present disclosure provides a pharmaceutical composition comprising the compound of the present disclosure or a pharmaceutically acceptable salt thereof, and a pharmaceutically acceptable excipient.

In a further aspect, the present disclosure provides a method for treating cancer, comprising administering an effective amount of a compound of the present disclosure or a pharmaceutically acceptable salt thereof or the pharmaceutical composition of the present disclosure to a subject in need thereof.

In a further aspect, the present disclosure provides use of the compound of the present disclosure or a pharmaceutically acceptable salt thereof or the pharmaceutical composition of the present disclosure in the manufacture of a medicament in the prevention or treatment of cancer.

In a further aspect, the present disclosure provides compounds of the present disclosure or a pharmaceutically acceptable salt thereof or the pharmaceutical composition of the present disclosure, for use in the treatment of cancer.

In a further aspect, the present disclosure provides a method for inhibiting ATR kinase in a subject in need thereof, comprising administering an effective amount of a compound of the present disclosure or a pharmaceutically acceptable salt thereof or the pharmaceutical composition of the present disclosure to the subject.

### DETAILED DESCRIPTION OF THE DISCLOSURE

Reference will now be made in detail to certain embodiments of the present disclosure, examples of which are illustrated in the accompanying structures and formulas. While the present disclosure will be described in conjunction with the enumerated embodiments, it will be understood that they are not intended to limit the present disclosure to those embodiments. On the contrary, the present disclosure is intended to cover all alternatives, modifications, and equivalents, which may be included within the scope of the present disclosure as defined by the claims. One skilled in the art will recognize many methods and materials similar or equivalent to those described herein, which could be used in the practice of the present disclosure. The present disclosure is in no way limited to the methods and materials described. In the event that one or more of the incorporated references and similar materials differs from or contradicts this application, including but not limited to defined terms, term usage, described techniques, or the like, the present disclosure controls. All references, patents, patent applications cited in the present disclosure are hereby incorporated by reference in their entireties.

It is appreciated that certain features of the present disclosure, which are, for clarity, described in the context of separate embodiments, can also be provided in combination in a single embodiment. Conversely, various features of the present disclosure, which are, for brevity, described in the context of a single embodiment, can also be provided separately or in any suitable sub-combination. It must be noted that, as used in the specification and the appended claims, the singular forms “a,” “an,” and “the” include plural forms of the same unless the context clearly dictates otherwise. Thus, for example, reference to “a compound” includes a plurality of compounds.

### Definitions

Definitions of specific functional groups and chemical terms are described in more detail below. For purposes of this disclosure, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, 2^{nd} Edition, University Science Books, Sausalito, 2006; Smith and March March’s Advanced Organic Chemistry, 6^{th} Edition, John Wiley & Sons, Inc., New York, 2007; Larock, Comprehensive Organic Transformations, 3^{rd} Edition, VCH Publishers, Inc., New York, 2018; Carruthers, Some Modern Methods of Organic Synthesis, 4^{th} Edition, Cambridge University Press, Cambridge, 2004; the entire contents of each of which are incorporated herein by reference.

At various places in the present disclosure, linking substituents are described. Where the structure clearly requires a linking group, the Markush variables listed for that group are understood to be linking groups. For example, if the structure requires a linking group and the Markush group definition for that variable lists “alkyl”, then it is understood that the “alkyl” represents a linking alkylene group.

When a bond to a substituent is shown to cross a bond connecting two atoms in a ring, then such substituent may be bonded to any atom in the ring. When a substituent is listed without indicating the atom via which such substituent is bonded to the rest of the compound of a given formula, then such substituent may be bonded via any atom in such formula. Combinations of substituents and/or variables are permissible, but only if such combinations result in stable compounds.

When any variable (e.g., Rⁱ) occurs more than one time in any constituent or formula for a compound, its definition at each occurrence is independent of its definition at every other occurrence. Thus, for example, if a group is shown to be substituted with 0-2 Rⁱ moieties, then the group may optionally be substituted with up to two Rⁱ moieties and Rⁱ at each occurrence is selected independently from the definition of Rⁱ. Also, combinations of substituents and/or variables are permissible, but only if such combinations result in stable compounds.

As used herein, the term “Cᵢ₋ⱼ” indicates a range of the carbon atoms numbers, wherein i and j are integers and the range of the carbon atoms numbers includes the endpoints (i.e. i and j) and each integer point in between, and wherein j is greater than i. For examples, C₁₋₆ indicates a range of one to six carbon atoms, including one carbon atom, two carbon atoms, three carbon atoms, four carbon atoms, five carbon atoms and six carbon atoms. In some embodiments, the term “C₁₋₁₂” indicates 1 to 12, particularly 1 to 10, particularly 1 to 8, particularly 1 to 6, particularly 1 to 5, particularly 1 to 4, particularly 1 to 3 or particularly 1 to 2 carbon atoms.

As used herein, the term “alkyl”, whether as part of another term or used independently, refers to a saturated linear or branched-chain hydrocarbon radical, which may be optionally substituted independently with one or more substituents described below. The term “Cᵢ₋ⱼ alkyl” refers to an alkyl having i to j carbon atoms. In some embodiments, alkyl groups contain 1 to 10 carbon atoms. In some embodiments, alkyl groups contain 1 to 9 carbon atoms. In some embodiments, alkyl groups contain 1 to 8 carbon atoms, 1 to 7 carbon atoms, 1 to 6 carbon atoms, 1 to 5 carbon atoms, 1 to 4 carbon atoms, 1 to 3 carbon atoms, or 1 to 2 carbon atoms. Examples of “C₁₋₁₀ alkyl” include, but are not limited to, methyl, ethyl, propyl, butyl, pentyl, hexyl, heptyl, octyl, nonyl, and decyl. Examples of “C₁₋₆ alkyl” are methyl, ethyl, propyl, isopropyl, n-butyl, i-butyl, s-butyl, t-butyl, n-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, and the like.

As used herein, the term “alkenyl”, whether as part of another term or used independently, refers to linear or branched-chain hydrocarbon radical having at least one carbon-carbon double bond, which may be optionally substituted independently with one or more substituents described herein, and includes radicals having “cis” and “trans” orientations, or alternatively, “E” and “Z” orientations. In some embodiments, alkenyl groups contain 2 to 12 carbon atoms. In some embodiments, alkenyl groups contain 2 to 11 carbon atoms. In some embodiments, alkenyl groups contain 2 to 11 carbon atoms, 2 to 10 carbon atoms, 2 to 9 carbon atoms, 2 to 8 carbon atoms, 2 to 7 carbon atoms, 2 to 6 carbon atoms, 2 to 5 carbon atoms, 2 to 4 carbon atoms, 2 to 3 carbon atoms, and in some embodiments, alkenyl groups contain 2 carbon atoms. Examples of alkenyl group include, but are not limited to, ethylenyl (or vinyl), propenyl (allyl), butenyl, pentenyl, 1-methyl-2 buten-1-yl, 5-hexenyl, and the like.

As used herein, the term “alkynyl”, whether as part of another term or used independently, refers to a linear or branched hydrocarbon radical having at least one carbon-carbon triple bond, which may be optionally substituted independently with one or more substituents described herein. In some embodiments, alkenyl groups contain 2 to 12 carbon atoms. In some embodiments, alkynyl groups contain 2 to 11 carbon atoms. In some embodiments, alkynyl groups contain 2 to 11 carbon atoms, 2 to 10 carbon atoms, 2 to 9 carbon atoms, 2 to 8 carbon atoms, 2 to 7 carbon atoms, 2 to 6 carbon atoms, 2 to 5 carbon atoms, 2 to 4 carbon atoms, 2 to 3 carbon atoms, and in some embodiments, alkynyl groups contain 2 carbon atoms. Examples of alkynyl group include, but are not limited to, ethynyl, 1-propynyl, 2-propynyl, and the like.

As used herein, the term “cycloalkyl”, whether as part of another term or used independently, refer to a monovalent non-aromatic, saturated or partially unsaturated monocyclic and polycyclic ring system, in which all the ring atoms are carbon and which contains at least three ring forming carbon atoms. In some embodiments, the cycloalkyl may contain 3 to 12 ring forming carbon atoms, 3 to 10 ring forming carbon atoms, 3 to 9 ring forming carbon atoms, 3 to 8 ring forming carbon atoms, 3 to 7 ring forming carbon atoms, 3 to 6 ring forming carbon atoms, 3 to 5 ring forming carbon atoms, 4 to 12 ring forming carbon atoms, 4 to 10 ring forming carbon atoms, 4 to 9 ring forming carbon atoms, 4 to 8 ring forming carbon atoms, 4 to 7 ring forming carbon atoms, 4 to 6 ring forming carbon atoms, 4 to 5 ring forming carbon atoms. Cycloalkyl groups may be saturated or partially unsaturated. Cycloalkyl groups may be substituted. In some embodiments, the cycloalkyl group may be a saturated cyclic alkyl group. In some embodiments, the cycloalkyl group may be a partially unsaturated cyclic alkyl group that contains at least one double bond or triple bond in its ring system. In some embodiments, the cycloalkyl group may be monocyclic or polycyclic. Examples of monocyclic cycloalkyl group include, but are not limited to, cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, cyclohexadienyl, cycloheptyl, cyclooctyl, cyclononyl, cyclodecyl, cycloundecyl and cyclododecyl. Examples of polycyclic cycloalkyl group include, but are not limited to, adamantyl, norbornyl, fluorenyl, spiro-pentadienyl, spiro[3.6]-decanyl, bicyclo[1,1,1]pentenyl, bicyclo[2,2,1]heptenyl, and the like.

As used herein, the term “cyano” refers to -CN.

As used herein, the term “halogen” refers to an atom selected from fluorine (or fluoro), chlorine (or chloro), bromine (or bromo) and iodine (or iodo).

As used herein, the term “haloalkyl” refers to an alkyl, as defined above, that is substituted by one or more halogens, as defined above. Examples of haloalkyl include, but are not limited to, trifluoromethyl, difluoromethyl, trichloromethyl, 2,2,2-trifluoroethyl, 1,2-difluoroethyl, 3-bromo-2-fluoropropyl, 1,2-dibromoethyl, and the like.

As used herein, the term “heteroatom” refers to nitrogen, oxygen, sulfur or phosphorus, and includes any oxidized form of nitrogen or sulfur, and any quaternized form of a basic nitrogen (including N-oxides).

As used herein, the term “heteroaryl”, whether as part of another term or used independently, refers to an aryl group having, in addition to carbon atoms, one or more heteroatoms. The heteroaryl group can be monocyclic. Examples of monocyclic heteroaryl include, but are not limited to, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, benzofuranyl and pteridinyl. The heteroaryl group also includes polycyclic groups in which a heteroaromatic ring is fused to one or more aryl, cycloaliphatic, or heterocyclyl rings, where the radical or point of attachment is on the heteroaromatic ring. Examples of polycyclic heteroaryl include, but are not limited to, indolyl, isoindolyl, benzothienyl, benzofuranyl, benzo[1,3]dioxolyl, dibenzofuranyl, indazolyl, benzimidazolyl, benzthiazolyl, quinolyl, isoquinolyl, dihydroquinolinyl, dihydroisoquinolinyl, tetrahydroquinolinyl, tetrahydroisoquinolinyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and the like.

As used herein, the term “heterocyclyl” refers to a saturated or partially unsaturated carbocyclyl group in which one or more ring atoms are heteroatoms independently selected from oxygen, sulfur, nitrogen, phosphorus, and the like, the remaining ring atoms being carbon, wherein one or more ring atoms may be optionally substituted independently with one or more substituents. In some embodiments, the heterocyclyl is a saturated heterocyclyl. In some embodiments, the heterocyclyl is a partially unsaturated heterocyclyl having one or more double bonds in its ring system. In some embodiments, the heterocyclyl may contains any oxidized form of carbon, nitrogen or sulfur, and any quaternized form of a basic nitrogen. “Heterocyclyl” also includes radicals wherein the heterocyclyl radicals are fused with a saturated, partially unsaturated, or fully unsaturated (i.e., aromatic) carbocyclic or heterocyclic ring. The heterocyclyl radical may be carbon linked or nitrogen linked where such is possible. In some embodiments, the heterocycle is carbon linked. In some embodiments, the heterocycle is nitrogen linked. For example, a group derived from pyrrole may be pyrrol-1-yl (nitrogen linked) or pyrrol-3-yl (carbon linked). Further, a group derived from imidazole may be imidazol-1-yl (nitrogen linked) or imidazol-3-yl (carbon linked).

In some embodiments, the term “3- to 12-membered heterocyclyl” refers to a 3- to 12-membered saturated or partially unsaturated monocyclic or polycyclic heterocyclic ring system having 1 to 3 heteroatoms independently selected from nitrogen, oxygen, or sulfur. The fused, spiro and bridged ring systems are also included within the scope of this definition. Examples of monocyclic heterocyclyl include, but are not limited to oxetanyl, 1,1-dioxothietanylpyrrolidyl, tetrahydrofuryl, tetrahydrothienyl, tetrahydropyranyl, pyrrolyl, furanyl, thienyl, pyrazolyl, imidazolyl, triazolyl, oxazolyl, thiazolyl, piperidyl, piperazinyl, piperidinyl, morpholinyl, pyridinyl, pyrazinyl, pyrimidinyl, pyridazinyl, triazinyl, pyridonyl, pyrimidonyl, pyrazinonyl, pyrimidonyl, pyridazonyl, pyrrolidinyl, triazinonyl, and the like. Examples of fused heterocyclyl include, but are not limited to, phenyl fused ring or pyridinyl fused ring, such as quinolinyl, isoquinolinyl, tetrahydroquinolinyl, tetrahydroisoquinolinyl, quinoxalinyl, quinolizinyl, quinazolinyl, azaindolizinyl, pteridinyl, chromenyl, isochromenyl, indolyl, isoindolyl, indolizinyl, indazolyl, purinyl, benzofuranyl, isobenzofuranyl, benzimidazolyl, benzothienyl, benzothiazolyl, carbazolyl, phenazinyl, phenothiazinyl, phenanthridinyl, imidazo[1,2-a]pyridinyl, [1,2,4]triazolo[4,3-a]pyridinyl, [1,2,3]triazolo[4,3-a]pyridinyl groups, and the like. Examples of spiro heterocyclyl include, but are not limited to, spiropyranyl, spirooxazinyl, and the like. Examples of bridged heterocyclyl include, but are not limited to, morphanyl, hexamethylenetetraminyl, 3-aza-bicyclo[3.1.0]hexane, 8-aza-bicyclo[3.2.1]octane, 1-aza-bicyclo[2.2.2]octane, 1,4-diazabicyclo[2.2.2]octane (DABCO), and the like.

As used herein, the term “hydroxyl” refers to —OH.

As used herein, the term “partially unsaturated” refers to a radical that includes at least one double or triple bond. The term “partially unsaturated” is intended to encompass rings having multiple sites of unsaturation, but is not intended to include aromatic (i.e., fully unsaturated) moieties.

As used herein, the term “substituted”, whether preceded by the term “optionally” or not, means that one or more hydrogens of the designated moiety are replaced with a suitable substituent. It will be understood that “substitution” or “substituted with” includes the implicit proviso that such substitution is in accordance with permitted valence of the substituted atom and that the substitution results in a stable or chemically feasible compound, e.g., which does not spontaneously undergo transformation such as by rearrangement, cyclization, elimination, etc. Unless otherwise indicated, an “optionally substituted” group may have a suitable substituent at each substitutable position of the group, and when more than one position in any given structure may be substituted with more than one substituent selected from a specified group, the substituent may be either the same or different at every position. It will be understood by those skilled in the art that substituents can themselves be substituted, if appropriate. Unless specifically stated as “unsubstituted”, references to chemical moieties herein are understood to include substituted variants. For example, reference to an “aryl” group or moiety implicitly includes both substituted and unsubstituted variants.

### Compounds

The present disclosure provides novel compounds of Formula (I) and pharmaceutically acceptable salts thereof, synthetic methods for making the compounds, pharmaceutical compositions containing them and various uses of the disclosed compounds.

In one aspect, the present disclosure provides a compound having Formula (I):

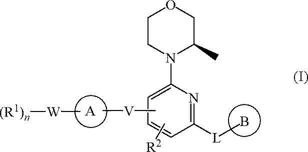

or a pharmaceutically acceptable salt thereof, wherein
Ring A is absent, 3- to 6-membered cycloalkyl, 5- to 6-membered heterocyclyl or 5-to 6-membered heteroaryl;
V is a direct bond, carbonyl or alkyl optionally substituted with one or more R^{c};
W and L are each independently a direct bond, —O—, —S—, or —N(R^{a})-;
R¹ is alkyl, cyano, —S(O)₂CH₃, or —S(O)(NH)CH₃;
R² is hydrogen, halogen or alkyl optionally substituted with one or more R^{b};
Ring B is

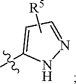

R⁵ is selected from the group consisting of hydrogen, halogen, hydroxyl, cyano, alkyl, alkenyl, alkynyl, heteroalkyl, heteroalkenyl, heteroalkynyl and haloalkyl;
R^{a} is hydrogen or alkyl;
R^{b} is hydroxyl or halogen;
R^{c} is hydroxyl, halogen or alkyl;
n is 0, 1, 2, or 3.

In some embodiments, V is a direct bond.

In some embodiments, V is carbonyl.

In some embodiments, V is alkyl optionally substituted with one or more R^{c}. In certain embodiments, V is C₁₋₆ alkyl, C₁₋₅ alkyl, C₁₋₄ alkyl or C₁₋₃ alkyl.

In some embodiments, Ring A is absent.

In some embodiments, Ring A is 3- to 6-membered cycloalkyl.

In certain embodiments, Ring A is cyclopropyl. In certain embodiments, Ring A is

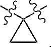

In certain embodiments, Ring A is cyclopentyl. In certain embodiments, Ring A is

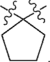

In certain embodiments, Ring A is cyclohexyl. In certain embodimens, Ring A is

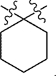

In some embodiments, Ring A is 5- to 6-membered heterocyclyl.

In certain embodiments, Ring A is 5-membered heterocyclyl containing at least one nitrogen atom. In certain embodiments, Ring A is 5-membered heterocyclyl containing at least two nitrogen atoms. In certain embodiments, Ring A is 5-membered heterocyclyl containing two nitrogen atoms.

In some embodiments, Ring A is pyrazolyl.

In certain embodiments, Ring A is 6-membered heterocyclyl.

In some embodiments, Ring A is tetrahydropyranyl.

In some embodiments, Ring A is 5- to 6-membered heteroaryl.

In certain embodiments, Ring A is 5- to 6-membered heteroaryl containing at least one nitrogen atom.

In certain embodiments, Ring A is 5-membered heteroaryl containing at least one nitrogen atom. In certain embodiments, Ring A is 5-membered heteroaryl containing at least one nitrogen atom and additional heteroatom(s) selected from O, N or S. In certain embodiments, Ring A is thiazolyl, triazolyl or isoxazolyl.

In certain embodiments, Ring A is 6-membered heteroaryl containing at least one nitrogen atom. In certain embodiments, Ring A is 6-membered heteroaryl containing at least one nitrogen atom and additional heteroatom(s) selected from O, N or S. In certain embodiments, Ring A is pyridyl.

In some embodiments, W is a direct bond.

In some embodiments, W is —N(R^{a})—.

In certain embodiments, W is —N(R^{a})—, and R^{a} is hydrogen.

In certain embodiments, W is —N(R^{a})—, and R^{a} is alkyl. In certain embodiments, W is —N(R^{a})—, and R^{a} is C₁₋₃ alkyl. In certain embodiments, W is —N(R^{a})—, and R^{a} is methyl.

In some embodiments, Ring A is 3- to 6-membered cycloalkyl, 5- to 6-membered heterocyclyl or 5- to 6-membered heteroaryl, and W is a direct bond.

In some embodiments, Ring A is absent, and W is -N(R^{a})-.

In some embodiments, Ring A is absent, W is —N(R^{a})—, and R^{a} is hydrogen.

In certain embodiments, Ring A is absent, W is —N(R^{a})—, and R^{a} is alkyl. In certain embodiments, Ring A is absent, W is —N(R^{a})—, and R^{a} is C₁₋₃ alkyl. In certain embodiments, Ring A is absent, W is —N(R^{a})—, and R^{a} is methyl.

In some embodiments, Ring A is absent, and W is a direct bond.

In some embodiments, R¹ is alkyl.

In some embodiments, R¹ is C₁₋₃ alkyl.

In some embodiments, R¹ is cyano.

In some embodiments, R¹ is hydroxyl.

In some embodiments, R¹ is —S(O)₂CH₃.

In some embodiments, R¹ is —S(O)(NH)CH₃.

In some embodiments, Ring A is absent, and R¹ is cyano or —S(O)₂CH₃.

In some embodiments, Ring A is 3- to 6-membered cycloalkyl, 5- to 6-membered heterocyclyl or 5- to 6-membered heteroaryl, and R¹ is alkyl, hydroxyl, —S(O)₂CH₃ or —S(O)(NH)CH₃.

In some embodiments, Ring A is cyclopropyl, cyclohexyl, tetrahydropyranyl, thiazolyl, pyridyl or isoxazolyl, and R¹ is —S(O)₂CH₃ or —S(O)(NH)CH₃.

In some embodiments, Ring A is cyclopropyl, R¹ is —S(O)₂CH₃ or —S(O)(NH)CH₃. In some embodiments, Ring A is

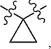

R¹ is —S(O)₂CH₃ or —S(O)(NH)CH₃.

In certain embodiments, Ring A is cyclopropyl, R¹ is —S(O)₂CH₃ or —S(O)(NH)CH₃, and n is 1. In certain embodiments, Ring A is

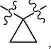

R¹ is —S(O)₂CH₃ or —S(O)(NH)CH₃, and n is 1.

In certain embodiments, Ring A is cyclopropyl, W is a direct bond, R¹ is —S(O)₂CH₃ or —S(O)(NH)CH₃, and n is 1. In certain embodiments, Ring A is

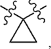

W is a direct bond, R¹ is —S(O)₂CH₃ or —S(O)(NH)CH₃, and n is 1.

In some embodiments,

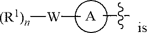

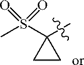

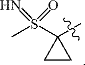

In some embodiments, Ring A is cyclopentyl, and R¹ is cyano. In certain embodiments, Ring A is

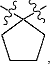

and R¹ is cyano. In certain embodiments, Ring A is

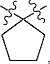

R¹ is cyano, and n is 1.

In certain embodiments, Ring A is cyclopentyl, W is a direct bond, R¹ is cyano, and n is 1. In certain embodiments, Ring A is

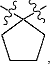

W is a direct bond, R¹ is cyano, and n is 1.

In some embodiments,

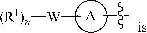

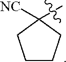

In some embodiments, Ring A is cyclohexyl, and R¹ is cyano. In certain embodiments, Ring A is

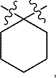

and R¹ is cyano. In certain embodiments, Ring A is

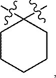

R¹ is cyano, and n is 1.

In certain embodiments, Ring A is cyclopentyl, W is a direct bond, R¹ is cyano, and n is 1. In certain embodiments, Ring A is

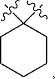

W is a direct bond, R¹ is cyano, and n is 1.

In some embodiments,

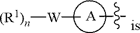

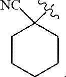

In some embodiments, Ring A is 5-membered heterocyclyl, and R¹ is alkyl.

In some embodiments, Ring A is pyrazolyl, isoxazolyl or triazolyl, and R¹ is C₁₋₃ alkyl.

In some embodiments, Ring A is pyrazolyl, isoxazolyl or triazolyl, and R¹ is methyl. In certain embodiments, Ring A is pyrazolyl, isoxazolyl or triazolyl, R¹ is methyl, and n is 2.

In some embodiments, Ring A is pyrazolyl, isoxazolyl or triazolyl, W is a direct bond, and R¹ is methyl. In certain embodiments, Ring A is pyrazolyl, isoxazolyl or triazolyl, W is a direct bond, R¹ is methyl, and n is 2.

In some embodiments,

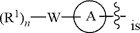

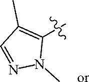

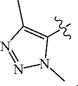

In some embodiments, Ring A is 5- to 6-membered heteroaryl, and R¹ is -S(O)₂CH₃.

In certain embodiments, Ring A is thiazolyl or pyridyl, and R¹ is —S(O)₂CH₃. In certain embodiments, Ring A is thiazolyl or pyridyl, R¹ is —S(O)₂CH₃, and n is 1.

In certain embodiments, Ring A is thiazolyl or pyridyl, W is a direct bond, and R¹ is —S(O)₂CH₃. In certain embodiments, Ring A is thiazolyl or pyridyl, W is a direct bond, R¹ is —S(O)₂CH₃, and n is 1.

In some embodiments,

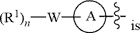

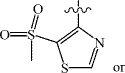

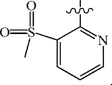

In some embodiments, L is a bond.

In some embodiments, L is —O—.

In some embodiments, L is —S—.

In some embodiments, L is —N(R^{a})—.

In certain embodiments, R^{a} is hydrogen.

In certain embodiments, R^{a} is C₁₋₃ alkyl.

In some embodiments, Ring B is

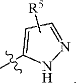

In some embodiments, L is —O—, —S— or —N(R^{a})—, and Ring B is

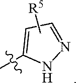

In certain embodiments, L is —O—, and Ring B is

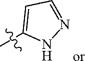

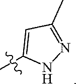

In certain embodiments, L is —S—, and Ring B is

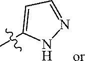

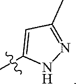

In certain embodiments, L is —N(R^{a})—, R^{a} is hydrogen, and Ring B is

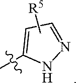

In certain embodiments, L is —N(R^{a})—, R^{a} is hydrogen, and Ring B is

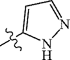

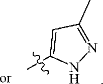

In some embodiments, R² is hydrogen.

In some embodiments, R² is halogen. In certain embodiments, R² is fluoro, chloro or bromo. In certain embodiments, R² is fluoro.

In some embodiments, R² is alkyl substituted with one or more R^{b}. In certain embodiments, R² is C₁₋₃ alkyl substituted with one or more R^{b}.

In some embodiments, R² is alkyl substituted with one or more R^{b}, and R^{b} is hydroxyl or fluoro. In certain embodiments, R² is C₁₋₃ alkyl substituted with one or more R^{b}, and R^{b} is hydroxyl or fluoro.

In some embodiments, R² is —CH₂OH or —CH₂F.

In some embodiments, the present disclosure provides compound having a Formula (II) or Formula (III):

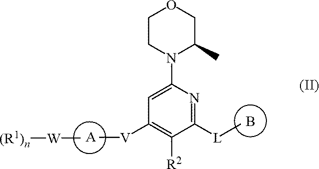

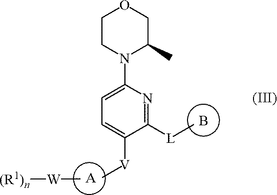

wherein V, W, L, Ring A, Ring B, R¹ and R² are as defined supra.

In some embodiments, the present disclosure provides a compound having a formula selected from the group consisting of:

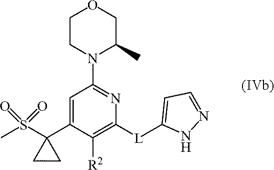

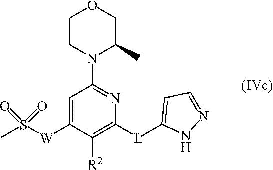

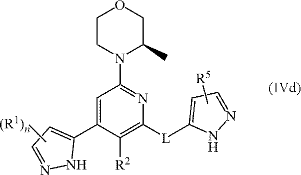

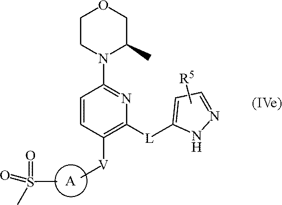

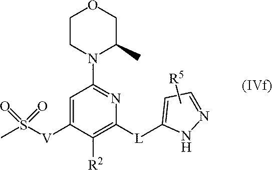

and

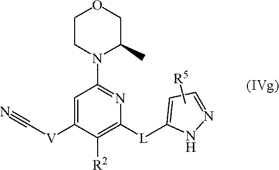

or a pharmaceutically acceptable salt thereof, wherein
U is O or NH;
V is a direct bond, carbonyl or alkyl optionally substituted with one or more R^{c};
W and L are each independently —O—, —S— or —N(R^{a})—;
R¹ is alkyl;
R² is hydrogen, halogen or alkyl substituted with one or more R^{b};
R³ is halogen;
R⁵ is hydrogen or alkyl;
R^{a} is hydrogen or alkyl;
R^{b} is hydroxyl or halogen; and
R^{c} is hydroxyl, halogen or alkyl.

In some embodiments, the present disclosure provides a compound having a formula selected from the group consisting of:

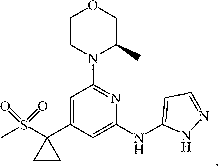

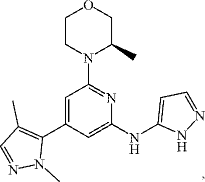

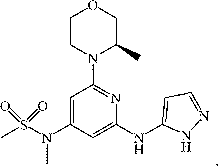

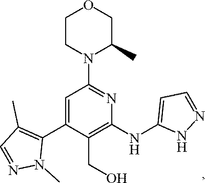

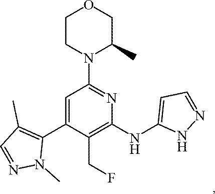

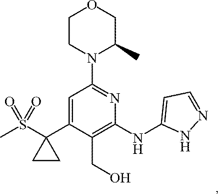

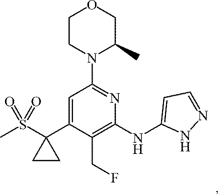

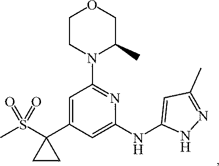

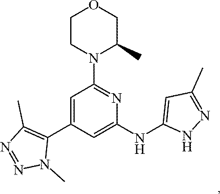

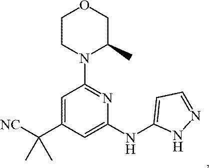

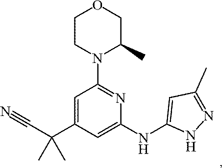

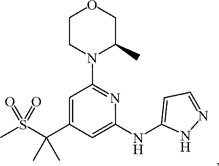

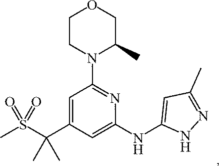

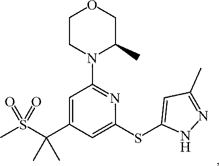

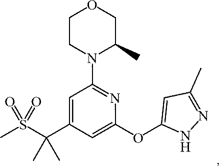

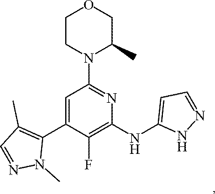

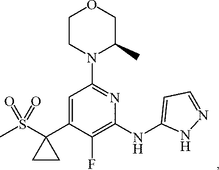

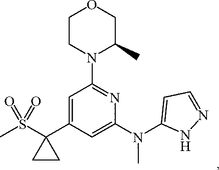

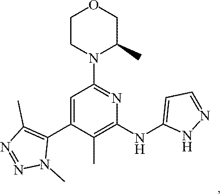

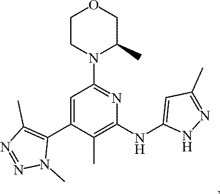

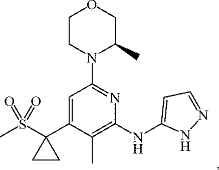

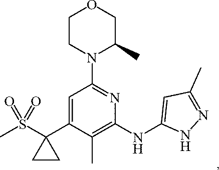

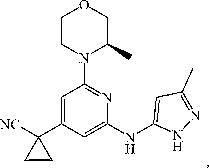

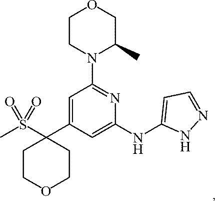

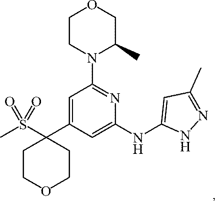

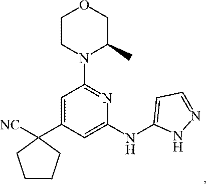

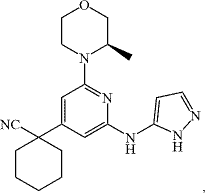

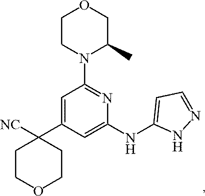

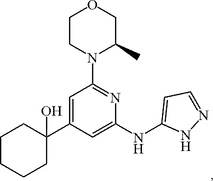

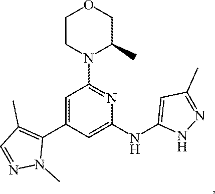

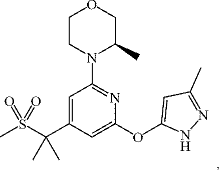

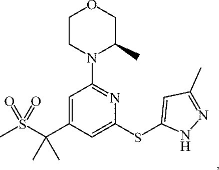

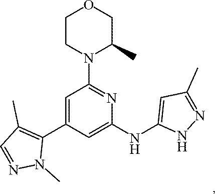

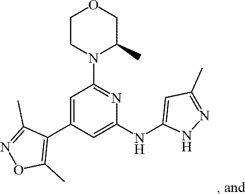

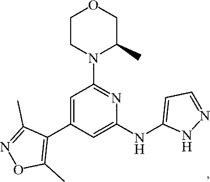

or a pharmaceutically acceptable salt thereof.

In a further aspect, the present disclosure provides a compound having Formula (V):

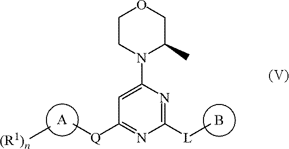

or a pharmaceutically acceptable salt thereof, wherein
Ring A is absent, 3- to 6-membered cycloalkyl, 5- to 6-membered heterocyclyl or 5- to 6-membered heteroaryl;
Q is a direct bond or alkyl optionally substituted with one or more R^{d};
L is —O—, —S— or —N(R^{a})—;
Ring B is

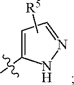

R^{d} is hydrogen or alkyl;
R^{d} is hydroxyl, halogen or alkyl;
R¹ is selected from the group consisting of cyano, hydroxyl, halogen, —S(O)₂CH₃, and —S(O)(NH)CH₃;
R⁵ is selected from the group consisting of hydrogen, halogen, hydroxyl, cyano, alkyl, alkenyl, alkynyl, heteroalkyl, heteroalkenyl, heteroalkynyl and haloalkyl;
n is 0, 1, 2, or 3.

In some embodiments, Q is a direct bond.

In some embodiments, Q is alkyl. In certain embodiments, Q is C₁₋₆ alkyl, C₁₋₅ alkyl, C₁₋₄ alkyl or C₁₋₃ alkyl.

In some embodiments, Ring A is 3- to 6-membered cycloalkyl. In certain embodiments, Ring A is cyclopropyl. In certain embodiments, Ring A is

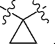

In some embodiments, Ring A is absent.

In some embodiments, Ring A is 5- to 6-membered heterocyclyl. In certain embodiments, Ring A is tetrahydropyranyl. In certain embodiments, Ring A is

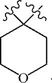

In some embodiments, Q is alkyl and Ring A is absent.

In some embodiments, Q is a direct bond and Ring A is 3- to 6-membered cycloalkyl or 5- to 6-membered heterocyclyl.

In some embodiments, R¹ is -S(O)₂CH₃ or —S(O)(NH)CH₃.

In some embodiments, R¹ is cyano, hydroxyl or halogen.

In some embodiments, Ring A is absent, 3- to 6-membered cycloalkyl or 5- to 6-membered heterocyclyl, and R¹ is —S(O)₂CH₃ or —S(O)(NH)CH₃.

In some embodiments, Ring A is absent or 3- to 6-membered cycloalkyl, and R¹ is cyano, hydroxyl or halogen.

In some embodiments, L is —O—.

In some embodiments, L is —S—.

In some embodiments, L is -N(R^{a})- and R^{a} is hydrogen.

In some embodiments, Ring B is

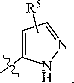

In some embodiments, R⁵ is hydrogen or alkyl.

In some embodiments, the present disclosure provides a compound having a formula selected from the group consisting of:

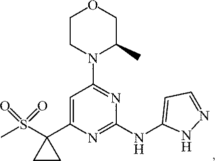

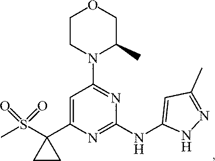

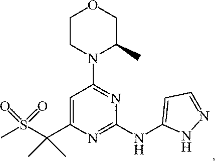

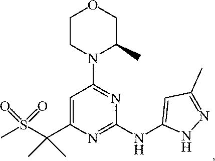

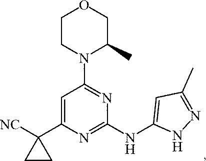

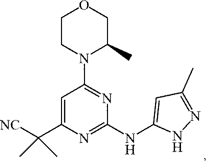

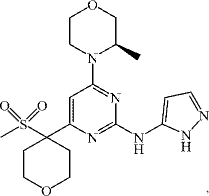

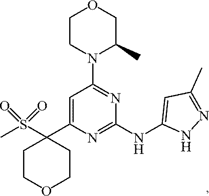

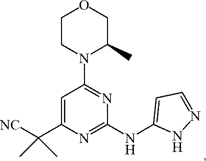

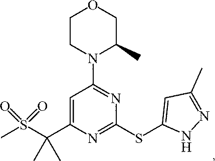

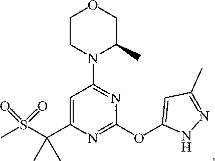

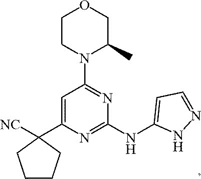

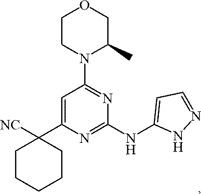

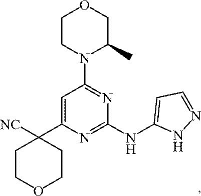

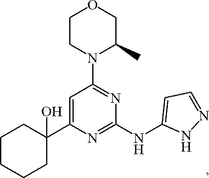

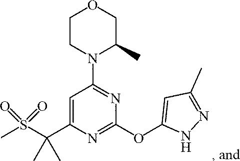

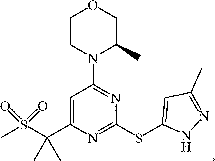

or a pharmaceutically acceptable salt thereof.

Exemplary compounds of the present disclosure are set forth in Table 1 below.

TABLE 1

| Compound No. | Compound structure |
|---|---|
| 1 | 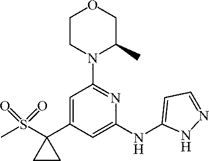 [SMILES: C[C@@H]1COCCN1c1cc(C2(S(C)(=O)=O)CC2)cc(Nc2ccn[nH]2)n1] |
| 2 | 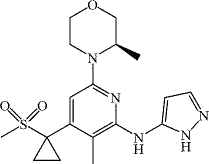 [SMILES: Cc1c(C2(S(C)(=O)=O)CC2)cc(N2CCOC[C@H]2C)nc1Nc1ccn[nH]1] |
| 3 | 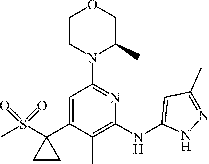 [SMILES: Cc1cc(Nc2nc(N3CCOC[C@H]3C)cc(C3(S(C)(=O)=O)CC3)c2C)[nH]n1] |
| 4 | 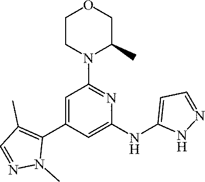 [SMILES: Cc1cnn(C)c1-c1cc(Nc2ccn[nH]2)nc(N2CCOC[C@H]2C)c1] |
| 5 | 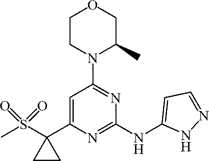 [SMILES: C[C@@H]1COCCN1c1cc(C2(S(C)(=O)=O)CC2)nc(Nc2ccn[nH]2)n1] |
| 6 | 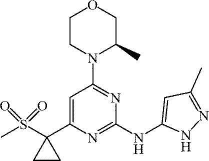 [SMILES: Cc1cc(Nc2nc(N3CCOC[C@H]3C)cc(C3(S(C)(=O)=O)CC3)n2)[nH]n1] |
| 7 | 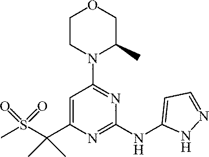 [SMILES: C[C@@H]1COCCN1c1cc(C(C)(C)S(C)(=O)=O)nc(Nc2ccn[nH]2)n1] |
| 8 | 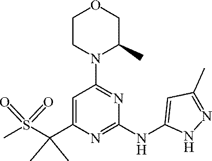 [SMILES: Cc1cc(Nc2nc(N3CCOC[C@H]3C)cc(C(C)(C)S(C)(=O)=O)n2)[nH]n1] |
| 9 | 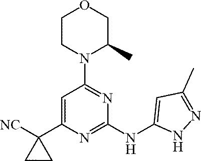 |
| 10 | 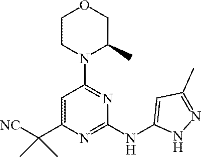 |
| 11 | 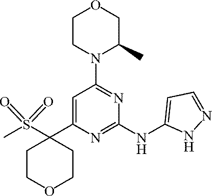 [SMILES: C[C@@H]1COCCN1c1cc(C2(S(C)(=O)=O)CCOCC2)nc(Nc2ccn[nH]2)n1] |
| 12 | 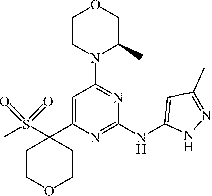 [SMILES: Cc1cc(Nc2nc(N3CCOC[C@H]3C)cc(C3(S(C)(=O)=O)CCOCC3)n2)[nH]n1] |
| 13 | 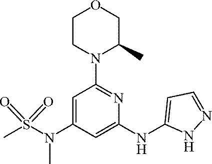 [SMILES: C[C@@H]1COCCN1c1cc(N(C)S(C)(=O)=O)cc(Nc2ccn[nH]2)n1] |
| 14 | 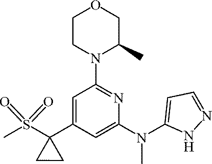 [SMILES: C[C@@H]1COCCN1c1cc(C2(S(C)(=O)=O)CC2)cc(N(C)c2ccn[nH]2)n1] |
| 15 | 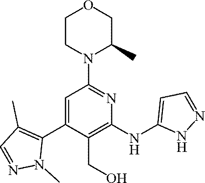 [SMILES: Cc1cnn(C)c1-c1cc(N2CCOC[C@H]2C)nc(Nc2ccn[nH]2)c1CO] |
| 16 | 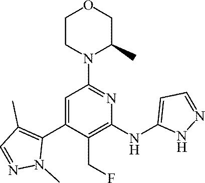 [SMILES: Cc1cnn(C)c1-c1cc(N2CCOC[C@H]2C)nc(Nc2ccn[nH]2)c1CF] |
| 17 | 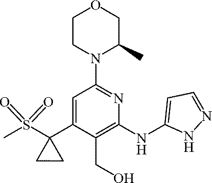 [SMILES: C[C@@H]1COCCN1c1cc(C2(S(C)(=O)=O)CC2)c(CO)c(Nc2ccn[nH]2)n1] |
| 18 | 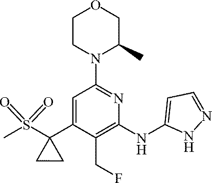 [SMILES: C[C@@H]1COCCN1c1cc(C2(S(C)(=O)=O)CC2)c(CF)c(Nc2ccn[nH]2)n1] |
| 19 | 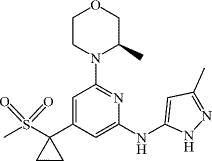 [SMILES: Cc1cc(Nc2cc(C3(S(C)(=O)=O)CC3)cc(N3CCOC[C@H]3C)n2)[nH]n1] |
| 20 | 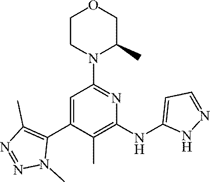 [SMILES: Cc1nnn(C)c1-c1cc(N2CCOC[C@H]2C)nc(Nc2ccn[nH]2)c1C] |
| 21 | 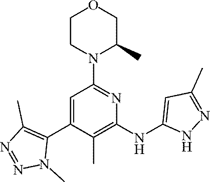 [SMILES: Cc1cc(Nc2nc(N3CCOC[C@H]3C)cc(-c3c(C)nnn3C)c2C)[nH]n1] |
| 22 | 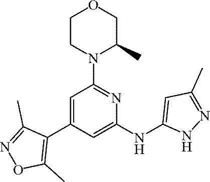 [SMILES: Cc1cc(Nc2cc(-c3c(C)noc3C)cc(N3CCOC[C@H]3C)n2)[nH]n1] |
| 23 | 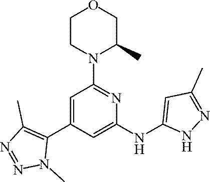 [SMILES: Cc1cc(Nc2cc(-c3c(C)nnn3C)cc(N3CCOC[C@H]3C)n2)[nH]n1] |
| 24 | 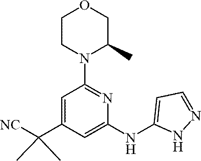 |
| 25 | 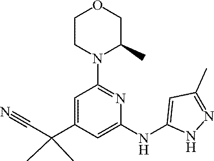 [SMILES: Cc1cc(Nc2cc(C(C)(C)C#N)cc(N3CCOC[C@H]3C)n2)[nH]n1] |
| 26 | 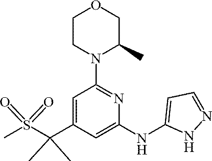 [SMILES: C[C@@H]1COCCN1c1cc(C(C)(C)S(C)(=O)=O)cc(Nc2ccn[nH]2)n1] |
| 27 | 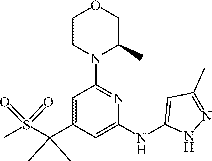 [SMILES: Cc1cc(Nc2cc(C(C)(C)S(C)(=O)=O)cc(N3CCOC[C@H]3C)n2)[nH]n1] |
| 28 | 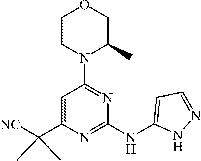 |
| 29 | 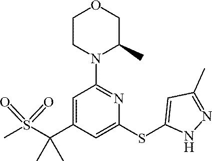 [SMILES: Cc1cc(Sc2cc(C(C)(C)S(C)(=O)=O)cc(N3CCOC[C@H]3C)n2)[nH]n1] |
| 30 | 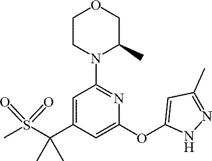 [SMILES: Cc1cc(Oc2cc(C(C)(C)S(C)(=O)=O)cc(N3CCOC[C@H]3C)n2)[nH]n1] |
| 31 | 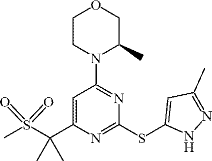 [SMILES: Cc1cc(Sc2nc(N3CCOC[C@H]3C)cc(C(C)(C)S(C)(=O)=O)n2)[nH]n1] |
| 32 | 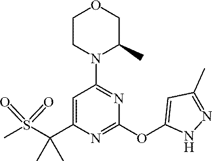 [SMILES: Cc1cc(Oc2nc(N3CCOC[C@H]3C)cc(C(C)(C)S(C)(=O)=O)n2)[nH]n1] |
| 33 | 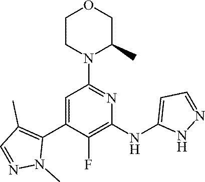 [SMILES: Cc1cnn(C)c1-c1cc(N2CCOC[C@H]2C)nc(Nc2ccn[nH]2)c1F] |
| 34 | 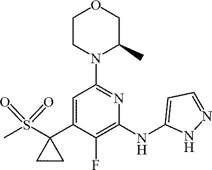 [SMILES: C[C@@H]1COCCN1c1cc(C2(S(C)(=O)=O)CC2)c(F)c(Nc2ccn[nH]2)n1] |
| 35 | 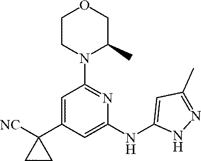 |
| 36 | 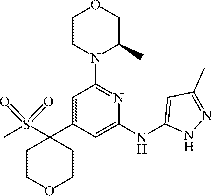 [SMILES: Cc1cc(Nc2cc(C3(S(C)(=O)=O)CCOCC3)cc(N3CCOC[C@H]3C)n2)[nH]n1] |
| 37 | 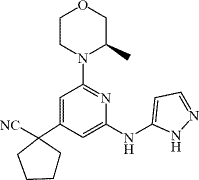 |
| 38 | 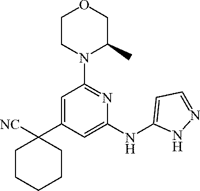 [SMILES: C[C@@H]1COCCN1c1cc(C2(C#N)CCCCC2)cc(Nc2ccn[nH]2)n1] |
| 39 | 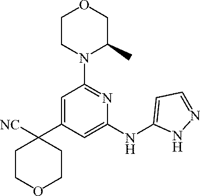 [SMILES: C[C@@H]1COCCN1c1cc(C2(C#N)CCOCC2)cc(Nc2ccn[nH]2)n1] |
| 40 | 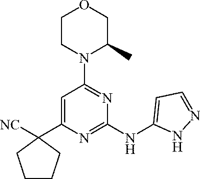 [SMILES: C[C@@H]1COCCN1c1cc(C2(C#N)CCCC2)nc(Nc2ccn[nH]2)n1] |
| 41 | 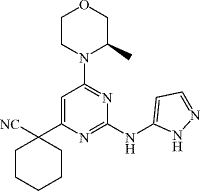 [SMILES: C[C@@H]1COCCN1c1cc(C2(C#N)CCCCC2)nc(Nc2ccn[nH]2)n1] |
| 42 | 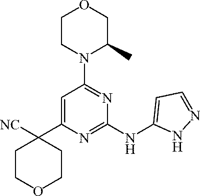 [SMILES: C[C@@H]1COCCN1c1cc(C2(C#N)CCOCC2)nc(Nc2ccn[nH]2)n1] |
| 43 | 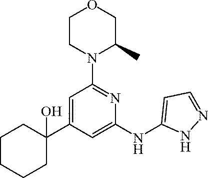 [SMILES: C[C@@H]1COCCN1c1cc(C2(O)CCCCC2)cc(Nc2ccn[nH]2)n1] |
| 44 | 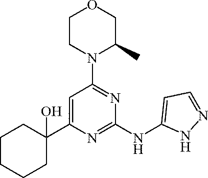 [SMILES: C[C@@H]1COCCN1c1cc(C2(O)CCCCC2)nc(Nc2ccn[nH]2)n1] |
| 45 | 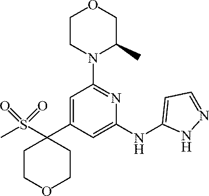 [SMILES: C[C@@H]1COCCN1c1cc(C2(S(C)(=O)=O)CCOCC2)cc(Nc2ccn[nH]2)n1] |
| 46 | 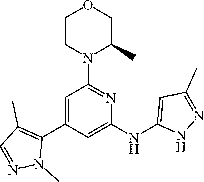 [SMILES: Cc1cc(Nc2cc(-c3c(C)cnn3C)cc(N3CCOC[C@H]3C)n2)[nH]n1] |
| 47 | 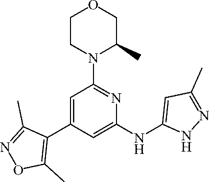 [SMILES: Cc1cc(Nc2cc(-c3c(C)noc3C)cc(N3CCOC[C@H]3C)n2)[nH]n1] |

Compounds provided herein are described with reference to both generic formulae and specific compounds. In addition, the compounds of the present disclosure may exist in a number of different forms or derivatives, including but not limited to prodrugs, soft drugs, active metabolic derivatives (active metabolites), and their pharmaceutically acceptable salts, all within the scope of the present disclosure.

As used herein, the term “prodrugs” refers to compounds or pharmaceutically acceptable salts thereof which, when metabolized under physiological conditions or when converted by solvolysis, yield the desired active compound. Prodrugs include, without limitation, esters, amides, carbamates, carbonates, ureides, solvates, or hydrates of the active compound. Typically, the prodrug is inactive, or less active than the active compound, but may provide one or more advantageous handling, administration, and/or metabolic properties. For example, some prodrugs are esters of the active compound; during metabolysis, the ester group is cleaved to yield the active drug. Also, some prodrugs are activated enzymatically to yield the active compound, or a compound which, upon further chemical reaction, yields the active compound. Prodrugs may proceed from prodrug form to active form in a single step or may have one or more intermediate forms which may themselves have activity or may be inactive. Preparation and use of prodrugs is discussed in T. Higuchi and V. Stella, “Pro-drugs as Novel Delivery Systems”, Vol. 14 of the A.C.S. Symposium Series, in Bioreversible Carriers in Drug Design, ed. Edward B. Roche, American Pharmaceutical Association and Pergamon Press, 1987; in Prodrugs: Challenges and Rewards, ed. V. Stella, R. Borchardt, M. Hageman, R. Oliyai, H. Maag, J. Tilley, Springer-Verlag New York, 2007, all of which are hereby incorporated by reference in their entirety.

As used herein, the term “soft drug” refers to compounds that exert a pharmacological effect but break down to inactive metabolites degradants so that the activity is of limited time. See, for example, “Soft drugs: Principles and methods for the design of safe drugs”, Nicholas Bodor, Medicinal Research Reviews, Vol. 4, No. 4, 449-469, 1984, which is hereby incorporated by reference in its entirety.

As used herein, the term “metabolite”, e.g., active metabolite overlaps with prodrug as described above. Thus, such metabolites are pharmacologically active compounds or compounds that further metabolize to pharmacologically active compounds that are derivatives resulting from metabolic process in the body of a subject. For example, such metabolites may result from oxidation, reduction, hydrolysis, amidation, deamidation, esterification, deesterification, enzymatic cleavage, and the like, of the administered compound or salt or prodrug. Of these, active metabolites are such pharmacologically active derivative compounds. For prodrugs, the prodrug compound is generally inactive or of lower activity than the metabolic product. For active metabolites, the parent compound may be either an active compound or may be an inactive prodrug.

Prodrugs and active metabolites may be identified using routine techniques know in the art. See, e.g., Bertolini et al, 1997, J Med Chem 40:2011-2016; Shan et al., J Pharm Sci 86:756-757; Bagshawe, 1995, DrugDev Res 34:220-230; Wermuth, supra.

As used herein, the term “pharmaceutically acceptable” indicates that the substance or composition is compatible chemically and/or toxicologically, with the other ingredients comprising a formulation, and/or the subjects being treated therewith.

As used herein, the term “pharmaceutically acceptable salt”, unless otherwise indicated, includes salts that retain the biological effectiveness of the free acids and bases of the specified compound and that are not biologically or otherwise undesirable. Contemplated pharmaceutically acceptable salt forms include, but are not limited to, mono, bis, tris, tetrakis, and so on. Pharmaceutically acceptable salts are non-toxic in the amounts and concentrations at which they are administered. The preparation of such salts can facilitate the pharmacological use by altering the physical characteristics of a compound without preventing it from exerting its physiological effect. Useful alterations in physical properties include lowering the melting point to facilitate transmucosal administration and increasing the solubility to facilitate administering higher concentrations of the drug.

Pharmaceutically acceptable salts include acid addition salts such as those containing sulfate, chloride, hydrochloride, fumarate, maleate, phosphate, sulfamate, acetate, citrate, lactate, tartrate, methanesulfonate, ethanesulfonate, benzenesulfonate, p-toluenesulfonate, cyclohexylsulfamate and quinate. Pharmaceutically acceptable salts can be obtained from acids such as hydrochloric acid, maleic acid, sulfuric acid, phosphoric acid, sulfamic acid, acetic acid, citric acid, lactic acid, tartaric acid, malonic acid, methanesulfonic acid, ethanesulfonic acid, benzenesulfonic acid, p-toluenesulfonic acid, cyclohexylsulfamic acid, fumaric acid, and quinic acid.

Pharmaceutically acceptable salts also include basic addition salts such as those containing benzathine, chloroprocaine, choline, diethanolamine, ethanolamine, t-butylamine, ethylenediamine, meglumine, procaine, aluminum, calcium, lithium, magnesium, potassium, sodium, ammonium, alkylamine, and zinc, when acidic functional groups, such as carboxylic acid or phenol are present. For example, see Remington’s Pharmaceutical Sciences, 19^{th}ed., Mack Publishing Co., Easton, PA, Vol. 2, p. 1457, 1995; “Handbook of Pharmaceutical Salts: Properties, Selection, and Use” by Stahl and Wermuth, Wiley-VCH, Weinheim, Germany, 2002. Such salts can be prepared using the appropriate corresponding bases.

Pharmaceutically acceptable salts can be prepared by standard techniques. For example, the free-base form of a compound can be dissolved in a suitable solvent, such as an aqueous or aqueous-alcohol solution containing the appropriate acid and then isolated by evaporating the solution. Thus, if the particular compound is a base, the desired pharmaceutically acceptable salt may be prepared by any suitable method available in the art, for example, treatment of the free base with an inorganic acid, such as hydrochloric acid, hydrobromic acid, sulfuric acid, nitric acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha-hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like.

Similarly, if the particular compound is an acid, the desired pharmaceutically acceptable salt may be prepared by any suitable method, for example, treatment of the free acid with an inorganic or organic base, such as an amine (primary, secondary or tertiary), an alkali metal hydroxide or alkaline earth metal hydroxide, or the like. Illustrative examples of suitable salts include organic salts derived from amino acids, such as L-glycine, L-lysine, and L-arginine, ammonia, primary, secondary, and tertiary amines, and cyclic amines, such as hydroxyethylpyrrolidine, piperidine, morpholine or piperazine, and inorganic salts derived from sodium, calcium, potassium, magnesium, manganese, iron, copper, zinc, aluminum and lithium.

It is also to be understood that the compounds of present disclosure can exist in unsolvated forms, solvated forms (e.g., hydrated forms), and solid forms (e.g., crystal or polymorphic forms), and the present disclosure is intended to encompass all such forms.

As used herein, the term “solvate” or “solvated form” refers to solvent addition forms that contain either stoichiometric or non-stoichiometric amounts of solvent. Some compounds have a tendency to trap a fixed molar ratio of solvent molecules in the crystalline solid state, thus forming a solvate. If the solvent is water the solvate formed is a hydrate; and if the solvent is alcohol, the solvate formed is an alcoholate. Hydrates are formed by the combination of one or more molecules of water with one molecule of the substance in which the water retains its molecular state as H₂O. Examples of solvents that form solvates include, but are not limited to, water, isopropanol, ethanol, methanol, DMSO, ethyl acetate, acetic acid, and ethanolamine.

As used herein, the terms “crystal form”, “crystalline form”, “polymorphic forms” and “polymorphs” can be used interchangeably, and mean crystal structures in which a compound (or a salt or solvate thereof) can crystallize in different crystal packing arrangements, all of which have the same elemental composition. Different crystal forms usually have different X-ray diffraction patterns, infrared spectral, melting points, density hardness, crystal shape, optical and electrical properties, stability and solubility. Recrystallization solvent, rate of crystallization, storage temperature, and other factors may cause one crystal form to dominate. Crystal polymorphs of the compounds can be prepared by crystallization under different conditions.

The compounds of present disclosure can comprise one or more asymmetric centers depending on substituent selection, and thus can exist in various stereoisomeric forms, e.g., enantiomers and/or diastereomers. For example, the compounds provided herein may have an asymmetric carbon center, and thus compounds provided herein may have either the (R) or (S) stereo-configuration at a carbon asymmetric center. Therefore, compounds of the present disclosure may be in the form of an individual enantiomer, diastereomer or geometric isomer, or may be in the form of a mixture of stereoisomers.

As used herein, the term “enantiomer” refers to two stereoisomers of a compound which are non-superimposable mirror images of one another. The term “diastereomer” refers to a pair of optical isomers which are not mirror images of one another. Diastereomers have different physical properties, e.g. melting points, boiling points, spectral properties, and reactivities.

Where a particular enantiomer is preferred, it may, in some embodiments be provided substantially free of the opposite enantiomer, and may also be referred to as “optically enriched”. “Optically enriched”, as used herein, means that the compound is made up of a significantly greater proportion of one enantiomer. In certain embodiments, the compound is made up of at least about 90% by weight of a preferred enantiomer. In other embodiments, the compound is made up of at least about 95%, 98%, or 99% by weight of a preferred enantiomer. Preferred enantiomers may be isolated from racemic mixtures by any method known to those skilled in the art, for example by chromatography or crystallization, by the use of stereochemically uniform starting materials for the synthesis or by stereoselective synthesis. Optionally a derivatization can be carried out before a separation of stereoisomers. The separation of a mixture of stereoisomers can be carried out at an intermediate step during the synthesis of a compound provided herein or it can be done on a final racemic product. Absolute stereochemistry may be determined by X-ray crystallography of crystalline products or crystalline intermediates which are derivatized, if necessary, with a reagent containing a stereogenic center of known configuration. Alternatively, absolute stereochemistry may be determined by Vibrational Circular Dichroism (VCD) spectroscopy analysis. See, for example, Jacques, et al., Enantiomers, Racemates and Resolutions (Wiley Interscience, New York, 1981); Wilen, S.H., et al., Tetrahedron 33:2725 (1977); Eliel, E.L. Stereochemistry of Carbon Compounds (McGraw-Hill, NY, 1962); Wilen, S.H. Tables of Resolving Agents and Optical Resolutions p. 268 (E.L. Eliel, Ed., Univ. of Notre Dame Press, Notre Dame, IN 1972).

In some embodiments, mixtures of diastereomers, for example mixtures of diastereomers enriched with 51% or more of one of the diastereomers, including for example 60% or more, 70% or more, 80% or more, or 90% or more of one of the diastereomers are provided.

In some embodiments, compounds provided herein may have one or more double bonds that can exist as either the Z or E isomer, unless otherwise indicated. The present disclosure additionally encompasses the compounds as individual isomers substantially free of other isomers and alternatively, as mixtures of various isomers, e.g., racemic mixtures of enantiomers.

The compounds of the present disclosure may also exist in different tautomeric forms, and all such forms are embraced within the scope of the present disclosure. The term “tautomer” or “tautomeric form” refers to structural isomers of different energies which are interconvertible via a low energy barrier. For example, proton tautomers (also known as prototropic tautomers) include interconversions via migration of a proton, such as keto-enol, amide-imidic acid, lactam-lactim, imine-enamine isomerizations and annular forms where a proton can occupy two or more positions of a heterocyclic system (for example, 1H- and 3H-imidazole, 1H-, 2H- and 4H- 1,2,4-triazole, 1H- and 2H- isoindole, and 1H- and 2H- pyrazole). Valence tautomers include interconversions by reorganization of some of the bonding electrons. Tautomers can be in equilibrium or sterically locked into one form by appropriate substitution. Compounds of the present disclosure identified by name or structure as one particular tautomeric form are intended to include other tautomeric forms unless otherwise specified.

The present disclosure is also intended to include all isotopes of atoms in the compounds. Isotopes of an atom include atoms having the same atomic number but different mass numbers. For example, unless otherwise specified, hydrogen, carbon, nitrogen, oxygen, phosphorous, sulfur, fluorine, chlorine, bromide or iodine in the compounds of present disclosure are meant to also include their isotopes, such as but not limited to ¹H, ²H, ³H, ¹¹C, ¹²C, ¹³C, ¹⁴C, ¹⁴N, ¹⁵N, ¹⁶O, ¹⁷O, ¹⁸O, ³¹P, ³²P, ³²S, ³³S, ³⁴S, ³⁶S, ¹⁷F, ¹⁸F, ¹⁹F, ³⁵Cl, ³⁷Cl, ⁷⁹Br, ⁸¹Br, ¹²⁴I, ¹²⁷I and ¹³¹I. In some embodiments, hydrogen includes protium, deuterium and tritium. In some embodiments, carbon includes ¹²C and ¹³C.

### Synthesis of Compounds

Synthesis of the compounds provided herein, including pharmaceutically acceptable salts thereof, are illustrated in the synthetic schemes in the examples. The compounds provided herein can be prepared using any known organic synthesis techniques and can be synthesized according to any of numerous possible synthetic routes, and thus these schemes are illustrative only and are not meant to limit other possible methods that can be used to prepare the compounds provided herein. Additionally, the steps in the Schemes are for better illustration and can be changed as appropriate. The embodiments of the compounds in examples were synthesized for the purposes of research and potentially submission to regulatory agencies.

The reactions for preparing compounds of the present disclosure can be carried out in suitable solvents, which can be readily selected by one skilled in the art of organic synthesis. Suitable solvents can be substantially non-reactive with the starting materials (reactants), the intermediates, or products at the temperatures at which the reactions are carried out, e.g. temperatures that can range from the solvent’s freezing temperature to the solvent’s boiling temperature. A given reaction can be carried out in one solvent or a mixture of more than one solvent. Depending on the particular reaction step, suitable solvents for a particular reaction step can be selected by one skilled in the art.

Preparation of compounds of the present disclosure can involve the protection and deprotection of various chemical groups. The need for protection and deprotection, and the selection of appropriate protecting groups, can be readily determined by one skilled in the art. The chemistry of protecting groups can be found, for example, in T. W. Greene and P. G. M. Wuts, Protective Groups in Organic Synthesis, 3rd Ed., Wiley & Sons, Inc., New York (1999), in P. Kocienski, Protecting Groups, Georg Thieme Verlag, 2003, and in Peter G.M. Wuts, Greene’s Protective Groups in Organic Synthesis, 5^{th} Edition, Wiley, 2014, all of which are incorporated herein by reference in its entirety.

Reactions can be monitored according to any suitable method known in the art. For example, product formation can be monitored by spectroscopic means, such as nuclear magnetic resonance spectroscopy (e.g.¹H or ¹³C), infrared spectroscopy, spectrophotometry (e.g. UV-visible), mass spectrometry, or by chromatographic methods such as high performance liquid chromatography (HPLC), liquid chromatography-mass spectroscopy (LCMS), or thin layer chromatography (TLC). Compounds can be purified by one skilled in the art by a variety of methods, including high performance liquid chromatography (HPLC) (“Preparative LC-MS Purification: Improved Compound Specific Method Optimization” Karl F. Blom, Brian Glass, Richard Sparks, Andrew P. Combs J. Combi. Chem. 2004, 6(6), 874-883, which is incorporated herein by reference in its entirety), and normal phase silica chromatography.

The known starting materials of the present disclosure can be synthesized by using or according to the known methods in the art, or can be purchased from commercial suppliers. Unless otherwise noted, analytical grade solvents and commercially available reagents were used without further purification.

Unless otherwise specified, the reactions of the present disclosure were all done under a positive pressure of nitrogen or argon or with a drying tube in anhydrous solvents, and the reaction flasks were typically fitted with rubber septa for the introduction of substrates and reagents via syringe. Glassware was oven dried and/or heat dried.

For illustrative purposes, the Examples section below shows synthetic route for preparing the compounds of the present disclosure as well as key intermediates. Those skilled in the art will appreciate that other synthetic routes may be used to synthesize the inventive compounds. Although specific starting materials and reagents are depicted, other starting materials and reagents can be easily substituted to provide a variety of derivatives and/or reaction conditions. In addition, many of the compounds prepared by the methods described below can be further modified in light of this disclosure using conventional chemistry well known to those skilled in the art.

### Pharmaceutical Compositions

In a further aspect, there is provided pharmaceutical compositions comprising one or more molecules or compounds of the present disclosure, or a pharmaceutically acceptable salt thereof.

In another aspect, there is provided pharmaceutical composition comprising one or more molecules or compounds of the present disclosure, or a pharmaceutically acceptable salt thereof, and at least one pharmaceutical acceptable excipient.

As used herein, the term “pharmaceutical composition” refers to a formulation containing the molecules or compounds of the present disclosure in a form suitable for administration to a subject.

As used herein, the term “pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used herein includes both one and more than one such excipient. The term “pharmaceutically acceptable excipient” also encompasses “pharmaceutically acceptable carrier” and “pharmaceutically acceptable diluent”.

The particular excipient used will depend upon the means and purpose for which the compounds of the present disclosure is being applied. Solvents are generally selected based on solvents recognized by persons skilled in the art as safe to be administered to a mammal including humans. In general, safe solvents are non-toxic aqueous solvents such as water and other non-toxic solvents that are soluble or miscible in water. Suitable aqueous solvents include water, ethanol, propylene glycol, polyethylene glycols (e.g., PEG 400, PEG 300), etc. and mixtures thereof.

In some embodiments, suitable excipients may include buffers such as phosphate, citrate and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

In some embodiments, suitable excipients may include one or more stabilizing agents, surfactants, wetting agents, lubricating agents, emulsifiers, suspending agents, preservatives, antioxidants, opaquing agents, glidants, processing aids, colorants, sweeteners, perfuming agents, flavoring agents and other known additives to provide an elegant presentation of the drug (i.e., a compound of the present disclosure or pharmaceutical composition thereof) or aid in the manufacturing of the pharmaceutical product (i.e., medicament). The active pharmaceutical ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington’s Pharmaceutical Sciences 16th edition, Osol, A. Ed. (1980). A “liposome” is a small vesicle composed of various types of lipids, phospholipids and/or surfactant which is useful for delivery of a drug (such as the compounds disclosed herein and, optionally, a chemotherapeutic agent) to a mammal including humans. The components of the liposome are commonly arranged in a bilayer formation, similar to the lipid arrangement of biological membranes.

The pharmaceutical compositions provided herein can be in any form that allows for the composition to be administered to a subject, including, but not limited to a human, and formulated to be compatible with an intended route of administration.

A variety of routes are contemplated for the pharmaceutical compositions provided herein, and accordingly the pharmaceutical composition provided herein may be supplied in bulk or in unit dosage form depending on the intended administration route. For example, for oral, buccal, and sublingual administration, powders, suspensions, granules, tablets, pills, capsules, gelcaps, and caplets may be acceptable as solid dosage forms, and emulsions, syrups, elixirs, suspensions, and solutions may be acceptable as liquid dosage forms. For injection administration, emulsions and suspensions may be acceptable as liquid dosage forms, and a powder suitable for reconstitution with an appropriate solution as solid dosage forms. For inhalation administration, solutions, sprays, dry powders, and aerosols may be acceptable dosage form. For topical (including buccal and sublingual) or transdermal administration, powders, sprays, ointments, pastes, creams, lotions, gels, solutions, and patches may be acceptable dosage form. For vaginal administration, pessaries, tampons, creams, gels, pastes, foams and spray may be acceptable dosage form.

The quantity of active ingredient in a unit dosage form of composition is a therapeutically effective amount and is varied according to the particular treatment involved. As used herein, the term “therapeutically effective amount” refers to an amount of a molecule, compound, or composition comprising the molecule or compound to treat, ameliorate, or prevent an identified disease or condition, or to exhibit a detectable therapeutic or inhibitory effect. The effect can be detected by any assay method known in the art. The precise effective amount for a subject will depend upon the subject’s body weight, size, and health; the nature and extent of the condition; the rate of administration; the therapeutic or combination of therapeutics selected for administration; and the discretion of the prescribing physician. Therapeutically effective amounts for a given situation can be determined by routine experimentation that is within the skill and judgment of the clinician.

In some embodiments, the pharmaceutical compositions of the present disclosure may be in a form of formulation for oral administration.

In certain embodiments, the pharmaceutical compositions of the present disclosure may be in the form of tablet formulations. Suitable pharmaceutically-acceptable excipients for a tablet formulation include, for example, inert diluents such as lactose, sodium carbonate, calcium phosphate or calcium carbonate, granulating and disintegrating agents such as corn starch or algenic acid; binding agents such as starch; lubricating agents such as magnesium stearate, stearic acid or talc; preservative agents such as ethyl or propyl p-hydroxybenzoate, and anti-oxidants, such as ascorbic acid. Tablet formulations may be uncoated or coated either to modify their disintegration and the subsequent absorption of the active ingredient within the gastrointestinal tract, or to improve their stability and/or appearance, in either case using conventional coating agents and procedures well known in the art.

In certain embodiments, the pharmaceutical compositions of the present disclosure may be in a form of hard gelatin capsules in which the active ingredient is mixed with an inert solid diluent, for example, calcium carbonate, calcium phosphate or kaolin, or as soft gelatin capsules in which the active ingredient is mixed with water or an oil such as peanut oil, liquid paraffin, or olive oil.

In certain embodiments, the pharmaceutical compositions of the present disclosure may be in the form of aqueous suspensions, which generally contain the active ingredient in finely powdered form together with one or more suspending agents, such as sodium carboxymethylcellulose, methylcellulose, hydroxypropylmethylcellulose, sodium alginate, polyvinyl-pyrrolidone, gum tragacanth and gum acacia; dispersing or wetting agents such as lecithin or condensation products of an alkylene oxide with fatty acids (for example polyoxethylene stearate), or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives (such as ethyl or propyl p-hydroxybenzoate, anti-oxidants (such as ascorbic acid), coloring agents, flavoring agents, and/or sweetening agents (such as sucrose, saccharine or aspartame).

In certain embodiments, the pharmaceutical compositions of the present disclosure may be in the form of oily suspensions, which generally contain suspended active ingredient in a vegetable oil (such as arachis oil, olive oil, sesame oil or coconut oil) or in a mineral oil (such as liquid paraffin). The oily suspensions may also contain a thickening agent such as beeswax, hard paraffin or cetyl alcohol. Sweetening agents such as those set out above, and flavoring agents may be added to provide a palatable oral preparation. These compositions may be preserved by the addition of an anti-oxidant such as ascorbic acid.

In certain embodiments, the pharmaceutical compositions of the present disclosure may be in the form of oil-in-water emulsions. The oily phase may be a vegetable oil, such as olive oil or arachis oil, or a mineral oil, such as for example liquid paraffin or a mixture of any of these. Suitable emulsifying agents may be, for example, naturally-occurring gums such as gum acacia or gum tragacanth, naturally-occurring phosphatides such as soya bean, lecithin, esters or partial esters derived from fatty acids and hexitol anhydrides (for example sorbitan monooleate) and condensation products of the said partial esters with ethylene oxide such as polyoxyethylene sorbitan monooleate. The emulsions may also contain sweetening, flavoring and preservative agents.

In certain embodiments, the pharmaceutical compositions provided herein may be in the form of syrups and elixirs, which may contain sweetening agents such as glycerol, propylene glycol, sorbitol, aspartame or sucrose, a demulcent, a preservative, a flavoring and/or coloring agent.

In some embodiments, the pharmaceutical compositions of the present disclosure may be in a form of formulation for injection administration.

In certain embodiments, the pharmaceutical compositions of the present disclosure may be in the form of a sterile injectable preparation, such as a sterile injectable aqueous or oleaginous suspension. This suspension may be formulated according to the known art using those suitable dispersing or wetting agents and suspending agents, which have been mentioned above. The sterile injectable preparation may also be a sterile injectable solution or suspension in a non-toxic parenterally acceptable diluent or solvent, such as a solution in 1,3-butanediol or prepared as a lyophilized powder. Among the acceptable vehicles and solvents that may be employed are water, Ringer’s solution and isotonic sodium chloride solution. In addition, sterile fixed oils may conventionally be employed as a solvent or suspending medium. For this purpose any bland fixed oil may be employed including synthetic mono- or diglycerides. In addition, fatty acids such as oleic acid may likewise be used in the preparation of injectables.

In some embodiments, the pharmaceutical compositions of the present disclosure may be in a form of formulation for inhalation administration.

In certain embodiments, the pharmaceutical compositions of the present disclosure may be in the form of aqueous and nonaqueous (e.g., in a fluorocarbon propellant) aerosols containing any appropriate solvents and optionally other compounds such as, but not limited to, stabilizers, antimicrobial agents, antioxidants, pH modifiers, surfactants, bioavailability modifiers and combinations of these. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols.

In some embodiments, the pharmaceutical compositions of the present disclosure may be in a form of formulation for topical or transdermal administration.

In certain embodiments, the pharmaceutical compositions provided herein may be in the form of creams, ointments, gels and aqueous or oily solutions or suspensions, which may generally be obtained by formulating an active ingredient with a conventional, topically acceptable excipients such as animal and vegetable fats, oils, waxes, paraffins, starch, tragacanth, cellulose derivatives, polyethylene glycols, silicones, bentonites, silicic acid, talc and zinc oxide, or mixtures thereof.

In certain embodiments, the pharmaceutical compositions provided herein may be formulated in the form of transdermal skin patches that are well known to those of ordinary skill in the art.

Besides those representative dosage forms described above, pharmaceutically acceptable excipients and carriers are generally known to those skilled in the art and are thus included in the present disclosure. Such excipients and carriers are described, for example, in “Remingtons Pharmaceutical Sciences” Mack Pub. Co., New Jersey (1991), in “Remington: The Science and Practice of Pharmacy”, Ed. University of the Sciences in Philadelphia, 21^{st} Edition, LWW (2005), which are incorporated herein by reference.

In some embodiments, the pharmaceutical compositions of the present disclosure can be formulated as a single dosage form. The amount of the compounds provided herein in the single dosage form will vary depending on the subject treated and particular mode of administration.

In some embodiments, the pharmaceutical compositions of the present disclosure can be formulated so that a dosage of between 0.001-1000 mg/kg body weight/day, for example, 0.01-800 mg/kg body weight/day, 0.01-700 mg/kg body weight/day, 0.01-600 mg/kg body weight/day, 0.01-500 mg/kg body weight/day, 0.01-400 mg/kg body weight/day, 0.01-300 mg/kg body weight/day, 0.1-200 mg/kg body weight/day, 0.1-150 mg/kg body weight/day, 0.1-100 mg/kg body weight/day, 0.5-100 mg/kg body weight/day, 0.5-80 mg/kg body weight/day, 0.5-60 mg/kg body weight/day, 0.5-50 mg/kg body weight/day, 1-50 mg/kg body weight/day, 1-45 mg/kg body weight/day, 1-40 mg/kg body weight/day, 1-35 mg/kg body weight/day, 1-30 mg/kg body weight/day, 1-25 mg/kg body weight/day of the compounds provided herein, or a pharmaceutically acceptable salt thereof, can be administered. In some instances, dosage levels below the lower limit of the aforesaid range may be more than adequate, while in other cases still larger doses may be employed without causing any harmful side effect, provided that such larger doses are first divided into several small doses for administration throughout the day. For further information on routes of administration and dosage regimes, see Chapter 25.3 in Volume 5 of Comprehensive Medicinal Chemistry (Corwin Hansch; Chairman of Editorial Board), Pergamon Press 1990, which is specifically incorporated herein by reference.

In some embodiments, the pharmaceutical compositions of the present disclosure can be formulated as short-acting, fast-releasing, long-acting, and sustained-releasing. Accordingly, the pharmaceutical formulations of the present disclosure may also be formulated for controlled release or for slow release.

In a further aspect, there is also provided veterinary compositions comprising one or more molecules or compounds of the present disclosure or pharmaceutically acceptable salts thereof and a veterinary carrier. Veterinary carriers are materials useful for the purpose of administering the composition and may be solid, liquid or gaseous materials which are otherwise inert or acceptable in the veterinary art and are compatible with the active ingredient. These veterinary compositions may be administered parenterally, orally or by any other desired route.

The pharmaceutical compositions or veterinary compositions may be packaged in a variety of ways depending upon the method used for administering the drug. For example, an article for distribution can include a container having deposited therein the compositions in an appropriate form. Suitable containers are well known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings. The compositions may also be packaged in unit-dose or multi-dose containers, for example sealed ampoules and vials, and may be stored in a freeze-dried (lyophilized) condition requiring only the addition of the sterile liquid carrier, for example water, for injection immediately prior to use. Extemporaneous injection solutions and suspensions are prepared from sterile powders, granules and tablets of the kind previously described.

In a further aspect, there is also provided pharmaceutical compositions comprise one or more compounds of the present disclosure, or a pharmaceutically acceptable salt thereof, as a first active ingredient, and a second active ingredient.

In some embodiments, the second active ingredient has complementary activities to the compound provided herein such that they do not adversely affect each other. Such ingredients are suitably present in combination in amounts that are effective for the purpose intended.

In some embodiments, the second active ingredient can include:
(i) antiproliferative/antineoplastic drugs and combinations thereof, as used in medical oncology such as alkylating agents (for example cis-platin, carboplatin, cyclophosphamide, nitrogen mustard, melphalan, chlorambucil, busulphan and nitrosoureas); antimetabolites (for example antifolates such as fluoropyrimidines like 5-fluorouracil and tegafur, raltitrexed, methotrexate, cytosine arabinoside, hydroxyurea and gemcitabine); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like paclitaxel and taxotere); and topoisomerase inhibitors (for example epipodophyllotoxins like etoposide and teniposide, amsacrine, topotecan and camptothecins);
(ii) cytostatic agents such as antioestrogens (for example tamoxifen, toremifene, raloxifene, droloxifene and iodoxyfene), oestrogen receptor down regulators (for example fulvestrant), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5a-reductase such as finasteride;
(iii) anti-invasion agents (for example c-Src kinase family inhibitors like 4-(6-chloro-2,3-methylenedioxyanilino)-7-[2-(4-methylpiperazin-1-yl)ethoxy]-5-tetrahydropyran-4-yloxyquinazoline (AZD0530) and N-(2-chloro-6-methylphenyl)-2-{6-[4-(2-hydroxyethyl)piperazin-1-yl]-2-methylpyrimidin-4-ylamino}thiazole-S-carboxamide (dasatinib, BMS-354825), and metalloproteinase inhibitors like marimastat and inhibitors of urokinase plasminogen activator receptor function);
(iv) inhibitors of growth factor function: for example such inhibitors include growth factor antibodies and growth factor receptor antibodies (for example the anti-erbB2 antibody trastuzumab [Herceptin™] and the anti-erbBl antibody cetuximab [C225]); such inhibitors also include, for example, tyrosine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine(gefitinib, ZD 1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)quinazolin-4-amine (CI 1033) and erbB2 tyrosine kinase inhibitors such as lapatinib), inhibitors of the hepatocyte growth factor family, inhibitors of the platelet-derived growth factor family such as imatinib, inhibitors of serine/threonine kinases (for example Ras/Raf signalling inhibitors such as farnesyl transferase inhibitors, for example sorafenib (BAY 43-9006)) and inhibitors of cell signalling through MEK and/or Akt kinases;
(v) antiangiogenic agents such as those which inhibit the effects of vascular endothelial growth factor, [for example the anti-vascular endothelial cell growth factor antibody bevacizumab (Avastin™) and VEGF receptor tyrosine kinase inhibitors such as 4-(4-bromo-2-fluoroanilino)-6-methoxy-7-(1-methylpiperidin-4-ylmethoxy)quinazoline (ZD6474; Example 2 within WO 01/32651), 4-(4-fluoro-2-methylindol-5-yloxy)-6-methoxy-7-(3-pyrrolidin-1-ylpropoxy)quinazoline (AZD2171; Example 240 within WO 00/47212), vatalanib (PTK787; WO 98/35985) and SU11248 (sunitinib; WO 01/60814), and compounds that work by other mechanisms (for example linomide, inhibitors of integrin αvβ3 function and angiostatin)];
(vi) vascular damaging agents such as combretastatin A4 and compounds disclosed in International Pat. Applications WO 99/02166, WO 00/40529, WO 00/41669, WO01/92224, WO 02/04434 and WO 02/08213;
(vii) antisense therapies, such as ISIS 2503, an anti-ras antisense agent;
(viii) gene therapy approaches, including approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; and
(ix) immunotherapeutic approaches, including ex-vivo and in-vivo approaches to increase the immunogenicity of patient tumour cells, such as transfection with cytokines such as interleukin 2, interleukin 4 or granulocyte -macrophage colony stimulating factor, approaches to decrease T-cell anergy, approaches using transfected immune cells such as cytokine-transfected dendritic cells, approaches using cytokine-trtnsfected tumour cell lines and approaches using anti-idiotypic antibodies.

### Method of Treatment of Disease

In an aspect, the present disclosure provides compounds of Formula (I) or pharmaceutically acceptable salts thereof, which are capable of inhibiting ATR kinase. The inhibitory properties of compounds of Formula (I) may be demonstrated using the test procedures set out herein.

Accordingly, the compounds of Formula (I) may be used in the treatment (therapeutic or prophylactic) of conditions or diseases in a subject which are mediated by ATR kinase.

As used herein, a “subject” refers to a human and a non-human animal. Examples of a non-human animal include all vertebrates, e.g., mammals, such as non-human primates (particularly higher primates), dog, rodent (e.g., mouse or rat), guinea pig, cat, and non-mammals, such as birds, amphibians, reptiles, etc. In a preferred embodiment, the subject is a human. In another embodiment, the subject is an experimental animal or animal suitable as a disease model.

In some embodiments, the compounds of Formula (I) can be used as antitumour agents. In some embodiments, the compounds of Formula (I) can be used as anti-proliferative, apoptotic and/or anti-invasive agents in the containment and/or treatment of solid and/or liquid tumour disease. In certain embodiments, the compounds of Formula (I) are useful in the prevention or treatment of those tumours which are sensitive to inhibition of ATR. In certain embodiments, the compounds of Formula (I) are useful in the prevention or treatment of those tumours which are mediated alone or in part by ATR.

In some embodiments, the compounds of Formula (I) are useful for the treatment of proliferative diseases, including malignant diseases such as cancer as well as non-malignant diseases such as inflammatory diseases, obstructive airways diseases, immune diseases or cardiovascular diseases.

In some embodiments, the compounds of Formula (I) are useful for the treatment of cancer, for example but not limited to, haematologic malignancies such as leukaemia, multiple myeloma, lymphomas such as Hodgkin’s disease, non-Hodgkin’s lymphomas (including mantle cell lymphoma), and myelodysplastic syndromes, and also solid tumours and their metastases such as breast cancer, lung cancer (non-small cell lung cancer (NSCL), small cell lung cancer (SCLC), squamous cell carcinoma), endometrial cancer, tumours of the central nervous system such as gliomas, dysembryoplastic neuroepithelial tumour, glioblastoma multiforme, mixed gliomas, medulloblastoma, retinoblastoma, neuroblastoma, germinoma and teratoma, cancers of the gastrointestinal tract such as gastric cancer, oesophagal cancer, hepatocellular (liver) carcinoma, cholangiocarcinomas, colon and rectal carcinomas, cancers of the small intestine, pancreatic cancers, cancers of the skin such as melanomas (in particular metastatic melanoma), thyroid cancers, cancers of the head and neck and cancers of the salivary glands, prostate, testis, ovary, cervix, uterus, vulva, bladder, kidney (including renal cell carcinoma, clear cell and renal oncocytoma), squamous cell carcinomas, sarcomas such as osteosarcoma, chondrosarcoma, leiomyosarcoma, soft tissue sarcoma, Ewing’s sarcoma, gastrointestinal stromal tumour (GIST), Kaposi’s sarcoma, and paediatric cancers such as rhabdomyosarcomas and neuroblastomas.

In some embodiments, the compounds of Formula (I) are useful for the treatment of autoimmune and/or inflammatory diseases, for example but not limited to, allergy, Alzheimer’s disease, acute disseminated encephalomyelitis, Addison’s disease, ankylosing spondylitis, antiphospholipid antibody syndrome, asthma, atherosclerosis, autoimmune hemolytic anemia, autoimmune hemolytic and thrombocytopenic states, autoimmune hepatitis, autoimmune inner ear disease, bullous pemphigoid, coeliac disease, chagas disease, chronic obstructive pulmonary disease, chronic Idiopathic thrombocytopenic purpura (ITP), churg-strauss syndrome, Crohn’s disease, dermatomyositis, diabetes mellitus type 1, endometriosis, Goodpasture’s syndrome (and associated glomerulonephritis and pulmonary hemorrhage), graves’ disease, guillain-barre syndrome, hashimoto’ s disease, hidradenitis suppurativa, idiopathic thrombocytopenic purpura, interstitial cystitis, irritable bowel syndrome, lupus erythematosus, morphea, multiple sclerosis, myasthenia gravis, narcolepsy, neuromyotonia, Parkinson’s disease, pemphigus vulgaris, pernicious anaemia, polymyositis, primary biliary cirrhosis, psoriasis, psoriatic arthritis, rheumatoid arthritis, schizophrenia, septic shock, scleroderma, Sjogren’s disease, systemic lupus erythematosus (and associated glomerulonephritis), temporal arteritis, tissue graft rejection and hyperacute rejection of transplanted organs, vasculitis (ANCA-associated and other vasculitides), vitiligo, and Wegener’s granulomatosis.

As used herein, the term “therapy” is intended to have its normal meaning of dealing with a disease in order to entirely or partially relieve one, some or all of its symptoms, or to correct or compensate for the underlying pathology, thereby achieving beneficial or desired clinical results. For purposes of this disclosure, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Therapy” can also mean prolonging survival as compared to expected survival if not receiving it. Those in need of therapy include those already with the condition or disorder as well as those prone to have the condition or disorder or those in which the condition or disorder is to be prevented. The term “therapy” also encompasses prophylaxis unless there are specific indications to the contrary. The terms “therapeutic” and “therapeutically” should be interpreted in a corresponding manner.

As used herein, the term “prophylaxis” or “prophylactic” is intended to have its normal meaning and includes primary prophylaxis to prevent the development of the disease and secondary prophylaxis whereby the disease has already developed and the patient is temporarily or permanently protected against exacerbation or worsening of the disease or the development of new symptoms associated with the disease.

The term “treatment” is used synonymously with “therapy”. Similarly the term “treat” can be regarded as “applying therapy” where “therapy” is as defined herein.

In a further aspect, the present disclosure provides use of the compound of the present disclosure or a pharmaceutically acceptable salt thereof or the pharmaceutical composition of the present disclosure for use in therapy, for example, for use in therapy associated with ATR kinase.

In a further aspect, the present disclosure provides use of the compound of the present disclosure or a pharmaceutically acceptable salt thereof or the pharmaceutical composition of the present disclosure, in the manufacture of a medicament for treating cancer.

In a further aspect, the present disclosure provides use of the compound of the present disclosure or a pharmaceutically acceptable salt thereof or the pharmaceutical composition of the present disclosure, in the manufacture of a medicament for treating cancer.

In another aspect, the present disclosure provides a compound of the present disclosure or a pharmaceutically acceptable salt thereof or a pharmaceutical composition of the present disclosure, for use in the treatment of cancer.

In some embodiments, the compounds of Formula (I) can be used further combination with other biologically active ingredients (such as, but not limited to, a second and different antineoplastic agent) and non-drug therapies (such as, but not limited to, surgery or radiation treatment). For instance, the compounds of Formula (I) can be used in combination with other pharmaceutically active compounds, or non-drug therapies, preferably compounds that are able to enhance the effect of the compounds of Formula (I). The compounds of Formula (I) can be administered simultaneously (as a single preparation or separate preparation) or sequentially to the other therapies. In general, a combination therapy envisions administration of two or more drugs/treatments during a single cycle or course of therapy.

In some embodiments, the compounds of Formula (I) are used in combination with one or more of traditional chemotherapeutic agents, which encompass a wide range of therapeutic treatments in the field of oncology. These agents are administered at various stages of the disease for the purposes of shrinking tumors, destroying remaining cancer cells left over after surgery, inducing remission, maintaining remission and/or alleviating symptoms relating to the cancer or its treatment.

In some embodiments, the compounds of Formula (I) are used in combination with one or more targeted anti-cancer agents that modulate protein kinases involved in various disease states.

In some embodiments, the compounds of Formula (I) are used in combination with one or more targeted anti-cancer agents that modulate non-kinase biological targets, pathway, or processes.

In some embodiments, the compounds of Formula (I) are used in combination with one or more of other anti-cancer agents that include, but are not limited to, gene therapy, RNAi cancer therapy, chemoprotective agents (e.g., amfostine, mesna, and dexrazoxane), drug-antibody conjugate(e.g brentuximab vedotin, ibritumomab tioxetan), cancer immunotherapy such as Interleukin-2, cancer vaccines(e.g., sipuleucel-T) or monoclonal antibodies (e.g., Bevacizumab, Alemtuzumab, Rituximab, Trastuzumab, etc).

In some embodiments, the compounds of Formula (I) are used in combination with one or more anti-inflammatory agent including but not limited to NSAIDs, non-specific and COX-2 specific cyclooxgenase enzyme inhibitors, gold compounds, corticosteroids, methotrexate, tumor necrosis factor receptor (TNF) receptors antagonists, immunosuppressants and methotrexate.

In some embodiments, the compounds of Formula (I) are used in combination with radiation therapy or surgeries. Radiation is commonly delivered internally (implantation of radioactive material near cancer site) or externally from a machine that employs photon (x-ray or gamma-ray) or particle radiation. Where the combination therapy further comprises radiation treatment, the radiation treatment may be conducted at any suitable time so long as a beneficial effect from the co-action of the combination of the therapeutic agents and radiation treatment is achieved.

Accordingly, in a further aspect, the present disclosure provides a method for treating diseases associated with ATR kinase in a subject in need thereof, comprising administering an effective amount of a compound of the present disclosure or a pharmaceutically acceptable salt thereof or the pharmaceutical composition of the present disclosure to the subject.

### EXAMPLES

For the purpose of illustration, the following examples are included. However, it is to be understood that these examples do not limit the present disclosure and are only meant to suggest a method of practicing the present disclosure. Persons skilled in the art will recognize that the chemical reactions described may be readily adapted to prepare a number of other compounds of the present disclosure, and alternative methods for preparing the compounds of the present disclosure are deemed to be within the scope of the present disclosure. For example, the synthesis of non-exemplified compounds according to the present disclosure may be successfully performed by modifications apparent to those skilled in the art, e.g., by appropriately protecting interfering groups, by utilizing other suitable reagents and building blocks known in the art other than those described, and/or by making routine modifications of reaction conditions. Alternatively, other reactions disclosed herein or known in the art will be recognized as having applicability for preparing other compounds of the present disclosure.

### Example 1

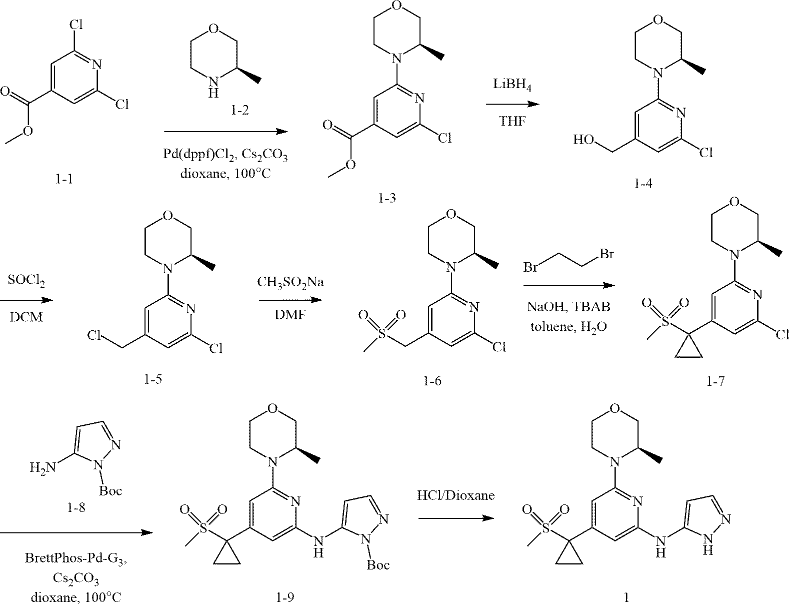

### Step 1: Methyl (R)-2-chloro-6-(3-methylmorpholino)isonicotinate (1-3)

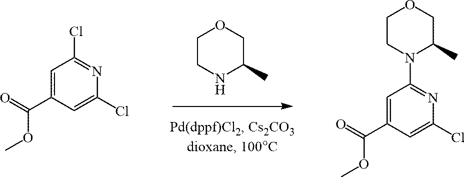

To a solution of methyl 2,6-dichloropyridine-4-carboxylate (2.5 g, 12.13 mmol) and (3R)-3-methylmorpholine (1.35 g, 13.35 mmol) in dioxane (50 mL) were added Cs₂CO₃ (7.91 g, 24.27 mmol) and Pd(dppf)Cl₂ (0.44 g, 0.61 mmol). The mixture was charged with N₂ twice, then stirred at 100° C. overnight. LC-MS showed the reaction was complete. After cooling to room temperature, the reaction mixture was diluted with EA (80 mL), then washed with water and brine, dried over anhydrous Na₂SO₄, filtered and concentrated. The residue was purified on flash column chromatography (Silica, 0~15% ethyl acetate in petroleum ether) to give the desired product (1.01 g, yield: 31%). LC-MS (ESI): m/z 271 [M+H]⁺. ¹H NMR (400 MHz, DMSO) δ 7.11 (d, J = 0.7 Hz, 1H), 7.00 (d, J = 0.7 Hz, 1H), 4.32 (dd, J = 6.7, 2.6 Hz, 1H), 3.96 - 3.88 (m, 2H), 3.87 (s, 3H), 3.72 (d, J = 11.4 Hz, 1H), 3.61 (dd, J = 11.5, 3.0 Hz, 1H), 3.46 (td, J= 11.9, 3.1 Hz, 1H), 3.13 (td, J= 12.7, 3.9 Hz, 1H), 1.15 (d, J= 6.7 Hz, 3H).

### Step 2. (R)-(2-chloro-6-(3-methylmorpholino)pyridin-4-yl)methanol (1-4)

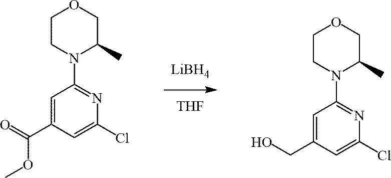 [SMILES: COC(=O)c1cc(Cl)nc(N2CCOC[C@H]2C)c1 ; "LiBH4" ; C[C@@H]1COCCN1c1cc(CO)cc(Cl)n1 ; "THF"]

To a solution of methyl 2-chloro-6-[(3R)-3-methylmorpholin-4-yl]pyridine-4-carboxylate (4.5 g, 16.62 mmol) in THF (40 mL) at 0° C. was added LiBH₄ solution (2.0 M in THF, 15.0 mL, 30.0 mmol). The resulting mixture was stirred at room temperature overnight under nitrogen atmosphere. LC-MS showed the reaction was complete. The reaction mixture was quenched with saturated NaHCO₃ aqueous solution and extracted with EA (60 mL×2). The combined organic layer was washed with brine, dried over anhydrous Na₂SO₄, filtered and concentrated in vacuo. The residue was purified by flash column chromatography (Silica, 0~50% ethyl acetate in petroleum ether) to give the title product (3.87 g, 96%). LC-MS(ESI): m/z 243 [M+H]⁺. ¹H NMR (400 MHz, CDCl₃) δ 6.58 (s, 1H), 6.46 (s, 1H), 4.62 (s, 2H), 4.31 - 4.23 (m, 1H), 3.99 (dd, J = 11.4, 3.8 Hz, 1H), 3.86 (dd, J = 13.1, 2.9 Hz, 1H), 3.78 (d, J = 11.3 Hz, 1H), 3.72 (dd, J= 11.4, 2.9 Hz, 1H), 3.61 - 3.54 (m, 1H), 3.21 (td, J = 12.7, 3.8 Hz, 1H), 1.89 (s, 1H), 1.24 (d, J = 6.7 Hz, 3H).

### Step 3. (R)-4-(6-chloro-4-(chloromethyl)pyridin-2-yl)-3-methylmorpholine (1-5)

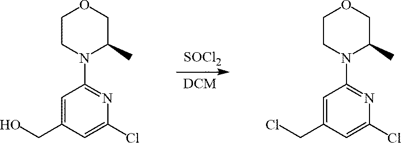

To a solution of {2-chloro-6-[(3R)-3-methylmorpholin-4-yl]pyridin-4-yl}methanol (4.0 g, 16.48 mmol) and DMF (0.05 mL, 0.65 mmol) in DCM (40 mL) at 0° C. was added SOCl₂ (10 mL, 137.8 mmol) dropwise. The resulting mixture was stirred at room temperature for 1 h under nitrogen atmosphere. LC-MS showed the reaction was complete. The reaction mixture was concentrated under vacuo to dryness. The residue was dissolved in DCM (50 mL), then washed with saturated NaHCO₃ aqueous solution and brine, dried over anhydrous Na₂SO₄, filtered and concentrated to give the desired product (4.08 g, yield: 95%). LC-MS(ESI): m/z 261 [M+H]⁺. ¹H NMR (400 MHz, CDCl₃) δ 6.62 (s, 1H), 6.43 (s, 1H), 4.41 (s, 2H), 4.26 (dd, J = 6.7, 2.6 Hz, 1H), 4.00 (dd, J= 11.4, 3.8 Hz, 1H), 3.86 (dd, J= 13.1, 3.0 Hz, 1H), 3.80 - 3.76 (m, 1H), 3.73 (dd, J= 11.4, 2.9 Hz, 1H), 3.61 - 3.54 (m, 1H), 3.22 (td, J = 12.7, 3.9 Hz, 1H), 1.26 (d, J = 6.7 Hz, 3H).

### Step 4. (R)-4-(6-chloro-4-((methylsulfonyl)methyl)pyridin-2-yl)-3-methyl morpholine (1-6)

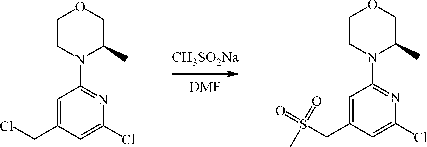

A mixture of (3R)-4-[6-chloro-4-(chloromethyl)pyridin-2-yl]-3-methylmorpholine (1.50 g, 5.74 mmol) and sodium methanesulfinate (1.17 g, 11.49 mmol) in DMF (20 mL) was stirred at room temperature overnight under nitrogen atmosphere. LC-MS showed the reaction was complete. The reaction mixture was diluted with H₂O and extracted with EA (60 mL×2). The combined organic layer was washed with brine, dried over anhydrous Na₂SO₄, filtered and concentrated. The residue was purified by flash column chromatography (Silica, 0~50% ethyl acetate in petroleum ether) to give the desired product (1.55 g, yield: 89%). LC-MS(ESI): m/z 305 [M+H]⁺. ¹H NMR (400 MHz, CDCl₃) δ 6.60 (s, 1H), 6.49 (s, 1H), 4.28 - 4.22 (m, 1H), 4.10 (s, 2H), 4.00 (dd, J= 11.5, 3.8 Hz, 1H), 3.90 (dd, J= 13.2, 2.8 Hz, 1H), 3.80 - 3.76 (m, 1H), 3.72 (dd, J = 11.4, 3.0 Hz, 1H), 3.61 - 3.54 (m, 1H), 3.23 (td, J = 12.7, 3.9 Hz, 1H), 2.85 (s, 3H), 1.26 (d, J = 6.7 Hz, 3H).

### Step 5. (R)-4-(6-chloro-4-(1-(methylsulfonyl)cyclopropyl)pyridin-2-yl)-3-methyl morpholine (1-7)

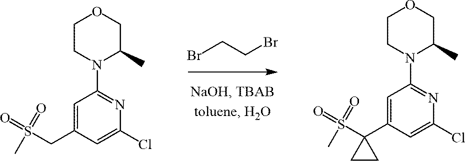

A mixture of (3R)-4-[6-chloro-4-(methanesulfonylmethyl)pyridin-2-yl]-3-methyl morpholine (1.55 g, 5.09 mmol), 1,2-dibromoethane (0.88 mL, 10.17 mmol), NaOH solution (10.0 M, 5.09 mL, 50.85 mmol) and TBAB (330 mg, 1.02 mmol) in toluene (50 mL) was stirred at 60° C. overnight under nitrogen atmosphere. LC-MS showed the reaction was complete. The reaction mixture was diluted with H₂O and extracted with EA (60 mL×2). The combined organic layer was washed with brine, dried over anhydrous Na₂SO₄, filtered and concentrated in vacuo. The residue was purified by flash column chromatography (Silica, 0~50% ethyl acetate in petroleum ether) to give the desired product (652 mg, yield: 39%). LC-MS(ESI): m/z 331 [M+H]⁺. ¹H NMR (400 MHz, CDCl₃) δ 6.70 (s, 1H), 6.67 (d, J = 0.9 Hz, 1H), 4.26 (d, J = 6.9 Hz, 1H), 4.00 (dd, J = 11.4, 3.8 Hz, 1H), 3.89 (dd, J = 13.2, 2.8 Hz, 1H), 3.78 (d, J= 11.4 Hz, 1H), 3.72 (dd, J = 11.4, 3.0 Hz, 1H), 3.58 (td, J = 11.9, 3.1 Hz, 1H), 3.22 (td, J = 12.7, 3.9 Hz, 1H), 2.83 (s, 3H), 1.88 - 1.76 (m, 2H), 1.26 (d, J = 6.6 Hz, 5H).

### Step 6. Tert-butyl (R)-5-((6-(3-methylmorpholino)-4-(1-(methylsulfonyl)cyclopropyl) pyridin-2-yl)amino)-1H-pyrazole-1-carboxylate (1-9)

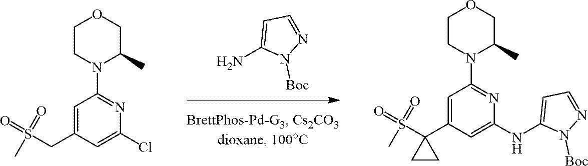

To a solution of (3R)-4-[6-chloro-4-(1-methanesulfonylcyclopropyl)pyridin-2-yl]-3-methylmorpholine (100 mg, 0.30 mmol) and tert-butyl 5-amino-1H-pyrazole-1-carboxylate (83 mg, 0.45 mmol) in dioxane (10 mL) were added BrettPhos-Pd-G3 catalyst (27 mg, 0.030 mmol) and Cs₂CO₃ (197 mg, 0.060 mmol). The mixture was charged with N₂ twice, then stirred at 100° C. for 4 h. LC-MS showed the reaction was complete. The reaction mixture was diluted with EA (50 mL), then washed with water and brine, dried over anhydrous Na₂SO₄, filtered and concentrated under vacuo. The residue was purified by flash column chromatography (Silica, 0~60% ethyl acetate in petroleum ether) to give the title product (59 mg, yield: 41%). LC-MS(ESI): m/z 478 [M+H]⁺.

### Step 7. (R)-6-(3-methylmorpholino)-4-(1-(methylsulfonyl)cyclopropyl)-N-(1H-pyrazol-5-yl)pyridin-2-amine (1)

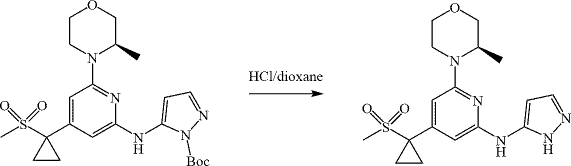

A mixture of tert-butyl 5-{ [4-(1-methanesulfonylcyclopropyl)-6-[(3R)-3-methyl morpholin-4-yl]pyridin-2-yl]amino }-IH-pyrazole-l-carboxylate (59 mg, 0.12 mol) and HCl solution (4 M in dioxane, 2 mL) in DCM (2 mL) was stirred at room temperature for 2 h. LC-MS showed the reaction was complete. The reaction mixture was concentrated under vacuo to dryness. The residue was purified by Prep-HPLC (C18, 20-95%, acetonitrile in H₂O with 0.1% HCOOH) to give the desired product (14.2 mg, yield 30%). LC-MS (ESI): m/z 378 [M+H]⁺. ¹H NMR (400 MHz, DMSO) δ 9.14 (s, 1H), 7.57 (d, J = 2.2 Hz, 1H), 6.60 (s, 1H), 6.28 (d, J= 2.2 Hz, 1H), 6.23 (s, 1H), 4.26 (d, J= 6.6 Hz, 1H), 3.94 (dd, J = 11.3, 3.3 Hz, 1H), 3.81 (d, J = 13.0 Hz, 1H), 3.73 (d, J = 11.2 Hz, 1H), 3.65 - 3.61 (m, 1H), 3.51 (s, 1H), 3.08 (d, J = 3.6 Hz, 1H), 2.95 (s, 3H), 1.57 (dd, J= 5.8, 4.0 Hz, 2H), 1.26 (dd, J= 6.3, 4.7 Hz, 2H), 1.14 (d, J = 6.6 Hz, 3H).

### Example 2

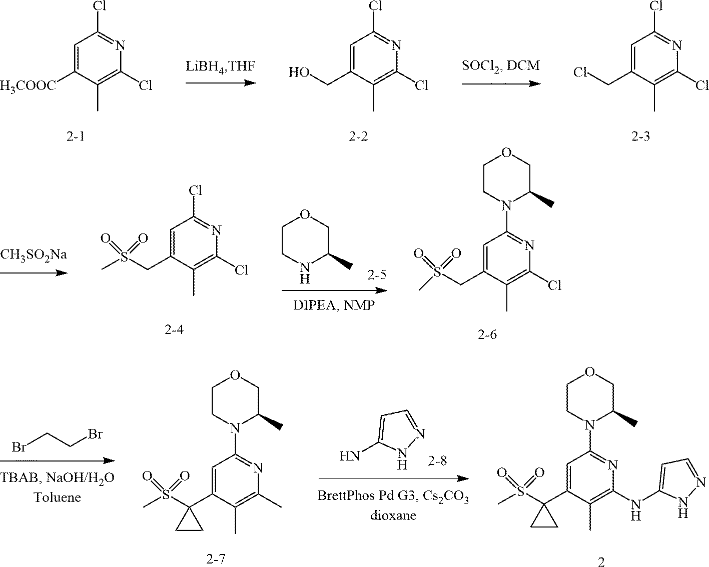

### Step 1. (2,6-dichloro-3-methylpyridin-4-yl)methanol (2-2)

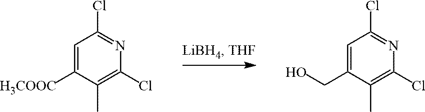

To a solution of ethyl 2,6-dichloro-3-methylpyridine-4-carboxylate (290 mg, 1.24 mmol) in anhydrous THF (5 mL) at 0° C. was added LiBH₄ solution (2.0 M in THF, 0.68 mL, 1.37 mmol) drop wise under N₂ atmosphere. The resulting mixture was stirred at 0° C. for 1 h. LC-MS showed the reaction was complete. The reaction mixture was quenched with saturated NH₄Cl aqueous solution and extracted with EA (50 mL). The organic layer was dried over anhydrous Na₂SO₄, filtered and concentrated under vacuo. The residue was purified by column chromatography on silica gel (PE : EA = 5:1, V/V) to give the desired product (220 mg, yield: 92%). LC/MS (ESI): m/z 192 [M+H]⁺.

### Step 2. 2,6-dichloro-4-(chloromethyl)-3-methylpyridine (2-3)

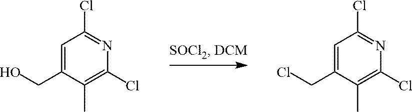

To a solution of (2,6-dichloro-3-methylpyridin-4-yl)methanol (220 mg, 1.14 mmol) and DMF (0.01 mL) in anhydrous DCM (5 mL) at 0° C. was added SOCl₂ (408 mg, 3.44 mmol) drop wise. The resulting mixture was stirred at room temperature for 1 h. LC-MS showed the reaction was complete. The reaction mixture was concentrated under reduced pressure. The residue was diluted with EA (40 mL), then washed with saturated NaHCO₃ aqueous solution and brine, dried over anhydrous Na₂SO₄, filtered and concentrated under vacuo. The residue was used in the next step without further purification (230 mg, yield: 95%). LC/MS (ESI): m/z 210/212 [M+H]⁺

### Step 3. 2,6-dichloro-3-methyl-4-((methylsulfonyl)methyl)pyridine Morpholine (2-4)

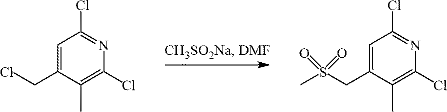

A mixture of 2,6-dichloro-4-(chloromethyl)-3-methylpyridine (259 mg, 1.23 mmol) and CH₃SO₂Na (253 mg, 2.48 mmol) in DMF (5 mL) was stirred at room temperature for 4 h. LC-MS showed the reaction was complete. The reaction mixture was diluted with EA (40 mL), then washed with water and brine, dried over anhydrous Na₂SO₄, filtered and concentrated. The residue was purified by column chromatography on silica gel (PE : EA = 1:1, V/V) to afford the desired product (270 mg, yield: 86%). LC/MS (ESI): m/z 254 [M+H]⁺.

### Step 4. (R)-4-(6-chloro-5-methyl-4-((methylsulfonyl)methyl)pyridin-2-yl)-3-methyl morpholine (2-6)

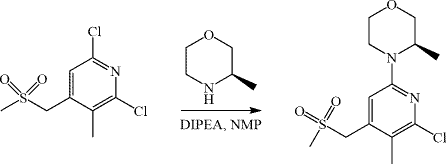

A mixture of 2,6-dichloro-4-(methanesulfonylmethyl)-3-methylpyridine (250 mg, 0.98 mmol), (3R)-3-methylmorpholine (399 mg, 3.94 mmol) and DIPEA (509 mg, 3.94 mmol) in NMP (3 mL) was stirred at 180° C. for 1 h under microwave irradiation. LC-MS showed the reaction was complete. The reaction mixture was diluted with EA (40 mL), then washed with water and brine, dried over anhydrous Na₂SO₄, filtered and concentrated. The residue was purified by column chromatography on silica gel (PE : EA = 1:1, V/V) to afford the desired product as a white solid (97 mg, yield: 30%). LC/MS (ESI): m/z 319 [M+H]⁺.

### Step 5. (R)-4-(6-chloro-5-methyl-4-(1-(methylsulfonyl)cyclopropyl)pyridin-2-yl)-3-methylmorpholine (2-7)

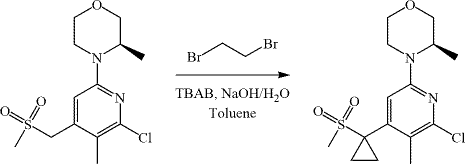

A mixture of (3R)-4-[6-chloro-4-(methanesulfonylmethyl)-5-methylpyridin-2-yl]-3-methylmorpholine (97 mg, 0.30 mmol), 1,2-dibromoethane (113 mg, 0.60 mmol), NaOH (10.0 M in H₂O, 0.3 mL, 3.05 mmol) and TBAB (19 mg, 0.06 mmol) in Toluene (4 mL) was stirred at 60° C. for 3 h. LC-MS showed the reaction was complete. The reaction mixture was diluted with EA (40 mL), then washed with water and brine, dried over anhydrous Na₂SO₄, filtered and concentrated. The residue was purified by column chromatography on silica gel (PE : EA = 1:1, V/V) to afford the desired product (29 mg, yield: 27%). LC/MS (ESI): m/z 345 [M+H]⁺.

### Step 6. (R)-3-methyl-6-(3-methylmorpholino)-4-(1-(methylsulfonyl)cyclopropyl)-N-(1H-pyrazol-5-yl)pyridin-2-amine (2)

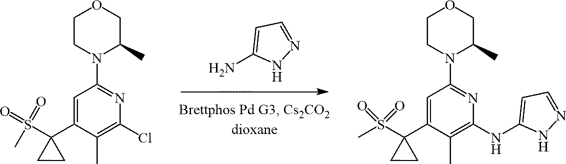

To a solution of (3R)-4-[6-chloro-4-(1-methanesulfonylcyclopropyl)-5-methylpyridin-2-yl]-3-methylmorpholine (30 mg, 0.08 mmol) and 1H-pyrazol-5-amine (14 mg, 0.16 mmol) in dioxane (1.5 mL) were added BrettPhos Pd G3 (8 mg, 0.01 mmol) and Cs₂CO₃ (85 mg, 0.26 mmol). The mixture was stirred at 110° C. for 10 h under N₂ atmosphere. LC-MS showed the reaction was complete. The reaction mixture was diluted with EA (40 mL), then washed with water and brine, dried over anhydrous Na₂SO₄, filtered and concentrated. The residue was purified by Prep-HPLC (C₁₈, 10-95%, MeOH in H₂O with 0.1% HCOOH) to give the desired product (17 mg, yield: 49%). LC/MS (ESI): m/z 392 [M+H]⁺. ¹H NMR (400 MHz, DMSO) δ 12.09 (s, 1H),7.94 (s, 1H), 7.52 (s, 1H), 6.40 (s, 1H), 6.24 (s, 1H), 4.20 (d, J = 5.0 Hz, 1H), 3.92 (dd, J = 11.2, 3.1 Hz, 1H), 3.71 (d, J = 11.4 Hz, 2H), 3.62 (dd, J = 11.2, 2.7 Hz, 1H), 3.47 (td, J = 11.8, 2.9 Hz, 1H), 3.06 - 2.98 (m, 1H), 2.95 (s, 3H), 2.17 (s, 3H), 1.87 (s, 1H), 1.52 (s, 1H), 1.24 (s, 2H), 1.09 (d, J = 6.1 Hz, 3H).

### Example 3

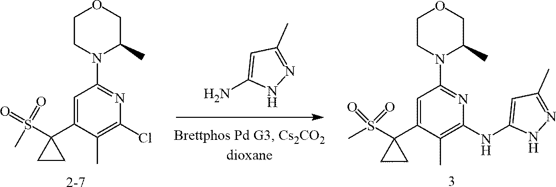

### Step 1. (R)-3-methyl-N-(3-methyl-1H-pyrazol-5-yl)-6-(3-methylmorpholino)-4-(1-(methylsulfonyl)cyclopropyl)pyridin-2-amine (3)

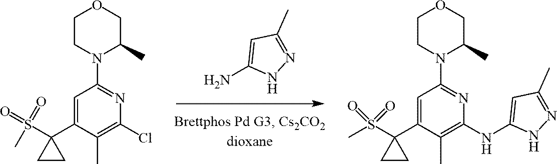

To a solution of (3R)-4-[6-chloro-4-(1-methanesulfonylcyclopropyl)-5-methylpyridin-2-yl]-3-methylmorpholine (50 mg, 0.14 mmol) and 3-methyl-1H-pyrazol-5-amine (28 mg, 0.28 mmol) in dioxane (2 mL) were added BrettPhos-Pd-G3 (13 mg, 0.01 mmol) and Cs₂CO₃ (142 mg, 0.43 mmol). The mixture was stirred at 110° C. for 10 h under N₂ atmosphere. LC-MS showed the reaction was complete. The reaction mixture was diluted with EA (40 mL), then washed with water and brine, dried over anhydrous Na₂SO₄, filtered and concentrated. The residue was purified by Prep-HPLC (C₁₈, 10-95%, MeOH in H₂O with 0.1% HCOOH) to give the desired product (54 mg, yield: 91%). LC/MS (ESI): m/z 406 [M+H]⁺. ¹H NMR (400 MHz, DMSO) δ 11.75 (s, 1H), 7.69 (s, 1H), 6.21 (s, 2H), 4.20 (s, 1H), 3.92 (d, J = 8.2 Hz, 1H), 3.71 (d, J = 11.3 Hz, 2H), 3.62 (d, J = 8.8 Hz, 1H), 3.47 (dd, J = 11.4, 8.9 Hz, 1H), 3.01 (t, J = 10.9 Hz, 1H), 2.94 (s, 3H), 2.17 (d, J = 15.2 Hz, 6H), 1.86 (s, 1H), 1.51 (s, 1H), 1.23 (s, 2H), 1.10 (d, J = 5.9 Hz, 3H).

### Example 4

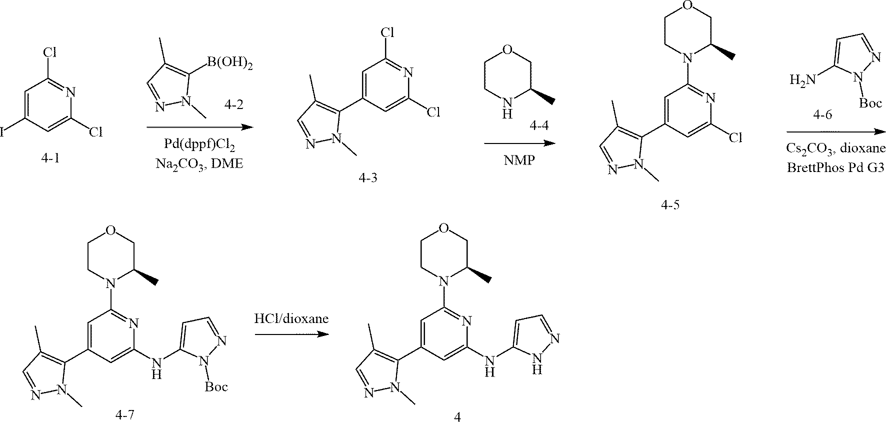

### Step 1. 2,6-dichloro-4-(1,4-dimethyl-1H-pyrazol-5-yl)pyridine (4-3)

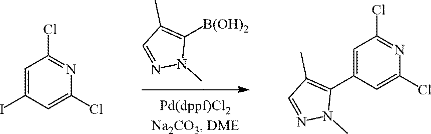

To a solution of 2,6-dichloro-4-iodopyridine (300 mg, 1.10 mmol) and 1,4-dimethyl-5-(tetramethyl-1,3,2-dioxaborolan-2-yl)-1H-pyrazole (267.6 mg, 1.21 mmol) in DME (10 mL) were added Na₂CO₃ (232.2 mg, 2.19 mmol) and Pd(dppf)Cl₂ (80.2 mg, 0.11 mmol). The mixture was charged with N₂ twice, then stirred at 90° C. for 4 h. LC-MS showed the reaction was complete. The reaction mixture was diluted with water (30 mL) and extracted with EA (40 mL × 2). The combined organic layer was washed with brine, dried over anhydrous Na₂SO₄, filtered and concentrated under vacuo. The residue was purified by Prep-TLC (PE:EA = 3:1, V/V) to afford the desired product (230 mg, yield: 86%). LC/MS (ESI) m/z: 243 [M+H]⁺.

### Step 2. (R)-4-(6-chloro-4-(1,4-dimethyl-1H-pyrazol-5-yl)pyridin-2-yl)-3-methyl morpholine (4-5)

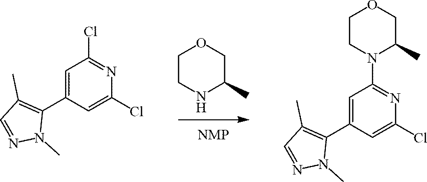

To a solution of 2,6-dichloro-4-(1,4-dimethyl-1H-pyrazol-5-yl)pyridine (230 mg, 0.95 mmol) in NMP (3 mL) was added (3R)-3-methylmorpholine (384.4 mg, 3.80 mmol). The reaction was stirred at 150° C. for 1 h under microwave irradiation. LC-MS showed the reaction was complete. The mixture was diluted with water (30 mL) and extracted with EA (40 mL × 2). The combined organic layer was washed with brine, dried over anhydrous Na₂SO₄, filtered and concentrated under vacuo. The residue was purified by flash column chromatography (Silica, 0~10% ethyl acetate in petroleum ether) to afford the desired product (150 mg, yield: 51%). LC/MS (ESI) m/z: 307 [M+H]⁺.

### Step 3. Tert-butyl (R)-5-((4-(1,4-dimethyl-1H-pyrazol-5-yl)-6-(3-methylmorpholino) pyridin-2-yl)amino)-1H-pyrazole-1-carboxylate (4-7)

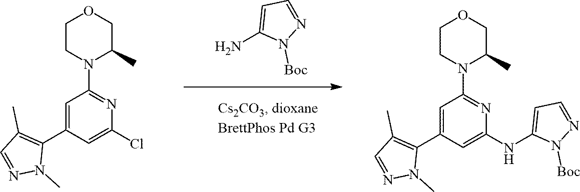

To a solution of (R)-4-(6-chloro-4-(1,4-dimethyl-1H-pyrazol-5-yl)pyridin-2-yl) -3-methylmorpholine (120 mg, 0.39 mmol) and tert-butyl 5-amino-1H-pyrazole-1-carboxylate (107.49 mg, 0.587 mmol) in dioxane (10 mL) were added CS₂CO₃ (637.2 mg, 1.96 mmol) and BrettPhos Pd G3 (35.46 mg, 0.04 mmol). The mixture was charged with N₂ twice, then stirred at 90° C. overnight. LC-MS showed the reaction was complete. The reaction was diluted with water (30 mL) and extracted with EA (40 mL×2). The combined organic layer was washed with brine, dried over anhydrous Na₂SO₄, filtered and concentrated under vacuo. The residue was purified by Prep-TLC (PE:EA = 2:1, V/V) to afford the desired product (80 mg, yield: 45%). LC/MS (ESI) m/z: 454 [M+H]⁺.

### Step 4. (R)-4-(1,4-dimethyl-1H-pyrazol-5-yl)-6-(3-methylmorpholino)-N-(1H-pyrazol-5-yl)pyridin-2-amine (4)

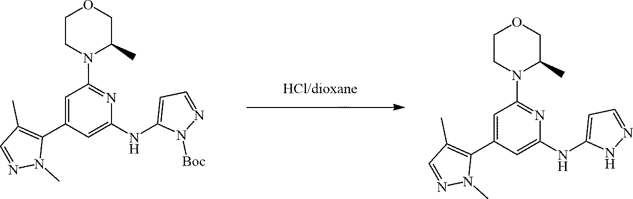

A mixture of tert-butyl (R)-5-((4-(1,4-dimethyl-1H-pyrazol-5-yl)-6-(3-methyl morpholino)pyridin-2-yl)amino)-1H-pyrazole-1-carboxylate (80 mg, 0.18 mmol) in HCl solution (4 M in dioxane, 2 mL) was stirred at room temperature overnight. LC-MS showed the reaction was complete. The reaction mixture was concentrated under vacuo to dryness. The residue was purified by Pre-HPLC (C₁₈, 20-95%, acetonitrile in H₂O with 0.1% TFA) to afford the desired product (20 mg, yield: 32%). LC/MS (ESI) m/z: 354 [M+H]⁺. ¹H NMR (400 MHz, DMSO) δ 9.05 (s, 1H), 7.55 (d, J = 2.2 Hz, 1H), 7.31 (s, 1H), 6.41 (s, 1H), 6.35 (d, J = 1.9 Hz, 1H), 6.00 (s, 1H), 4.30 (d, J = 6.8 Hz, 1H), 3.97 - 3.84 (m, 2H), 3.74 (s, 3H), 3.71 (s, 1H), 3.63(dd, J = 11.3, 2.8 Hz, 1H), 3.52 - 3.45 (m, 1H), 3.11 - 3.03 (m, 1H), 1.99 (s, 3H), 1.15 (d, J = 6.6 Hz, 3H).

### Example 5

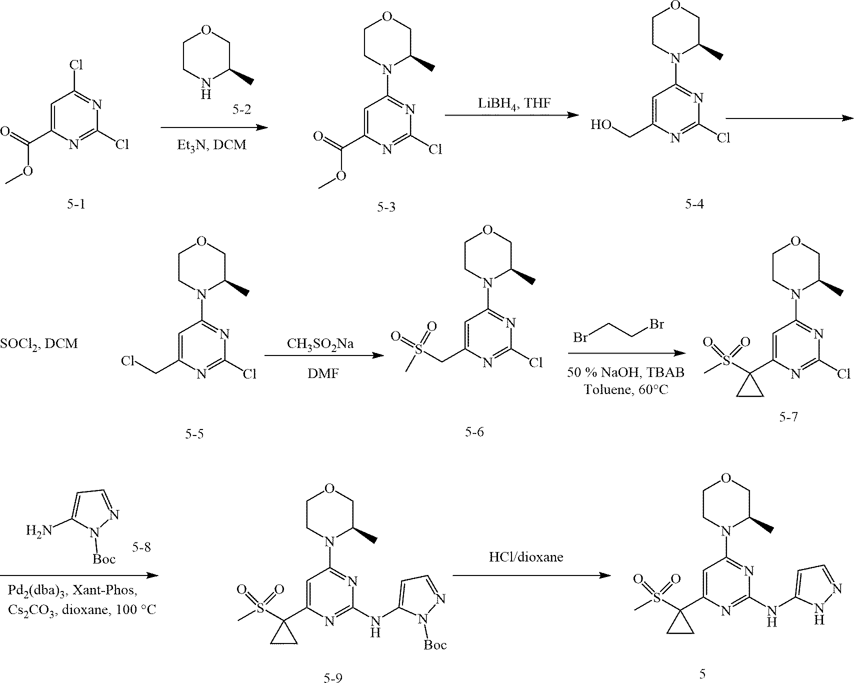

### Step 1. methyl(R)-2-chloro-6-(3-methylmorpholino)pyrimidine-4-carboxylate (5-3)

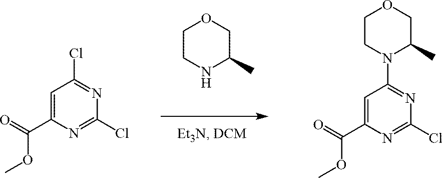

A mixture of methyl 2,6-dichloropyrimidine-4-carboxylate (1.5 g, 7.24 mmol), (3R)-3-methylmorpholine (732 mg, 7.24 mmol) and TEA (1.47 g, 14.52 mmol) in DCM (30 mL) was stirred at room temperature for 16 h. LC-MS showed the reaction was complete. The reaction mixture was diluted with DCM (20 mL), then washed with water and brine, dried over anhydrous Na₂SO₄, filtered and concentrated. The residue was purified by column chromatography on silica gel (PE : EA = 3:1, V/V) to afford the desired product (1.55 g, yield: 78%). LC/MS( ESI): m/z 272 [M+H]⁺.

### Step 2. (R)-(2-chloro-6-(3-methylmorpholino)pyrimidin-4-yl)methanol (5-4)

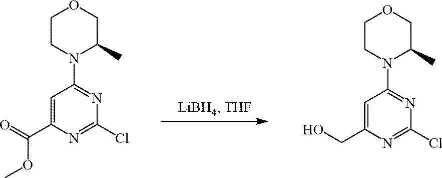

To a solution of methyl 2-chloro-6-[(3R)-3-methylmorpholin-4-yl]pyrimidine-4-carboxylate (1 g, 3.67 mmol) in anhydrous THF (20 mL) at 0° C. was added LiBH₄ solution (2.0 M in THF, 3.7 mL, 7.34 mmol) dropwise under N₂ atmosphere. The resulting mixture was stirred at 0° C. for 1 h. LC-MS showed the reaction was complete. The reaction mixture was quenched with saturated NH₄Cl aqueous solution and extracted with EA (50 mL). The combined organic layer was dried over anhydrous Na₂SO₄, filtered and concentrated under vacuo. The residue was purified by column chromatography on silica gel (PE : EA = 1:1, V/V) to give the desired product (800 mg, yield: 89%). LC/MS (ESI): m/z 244 [M+H]⁺.

### Step 3. (R)-4-(2-chloro-6-(chloromethyl)pyrimidin-4-yl)-3-methylmorpholine (5-5)

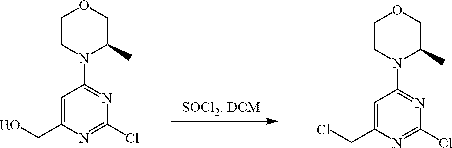

To a solution of {2-chloro-6-[(3R)-3-methylmorpholin-4-yl]pyrimidin-4-yl}methanol (800 mg, 3.28 mmol) and DMF (0.01 mL) in anhydrous DCM (20 mL) at 0° C. was added SOCl₂ (1.17 g, 9.84 mmol) dropwise. The resulting mixture was stirred at room temperature for 1 h. LC-MS showed the reaction was complete. The reaction mixture was concentrated under reduced pressure. The residue was dissolved in EA (40 mL), then washed with saturated NaHCO₃ aqueous solution and brine, dried over anhydrous Na₂SO₄, filtered and concentrated under vacuo. The residue was used in the next step without further purification (800 mg, yield: 93%). LC/MS (ESI): m/z 262/264 [M+H]⁺.

### Step 4. (R)-4-(2-chloro-6-((methylsulfonyl)methyl)pyrimidin-4-yl)-3-methyl morpholine (5-6)

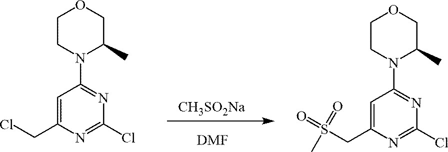

A mixture of (3R)-4-[2-chloro-6-(chloromethyl)pyrimidin-4-yl]-3-methylmorpholine (535 mg, 2.04 mmol) and CH₃SO₂Na (418 mg, 4.10 mmol) in DMF (10 mL) was stirred at room temperature for 16 h. LC-MS showed the reaction was complete. The reaction mixture was diluted with EA (40 mL), then washed with water and brine, dried over anhydrous Na₂SO₄, filtered and concentrated. The residue was purified by column chromatography on silica gel (PE : EA = 2:1, V/V) to afford the desired product (560 mg, yield: 90%). LC/MS( ESI): m/z 306 [M+H]⁺.

### Step 5. (R)-4-(2-chloro-6-(1-(methylsulfonyl)cyclopropyl)pyrimidin-4-yl)-3-methyl morpholine (5-7)

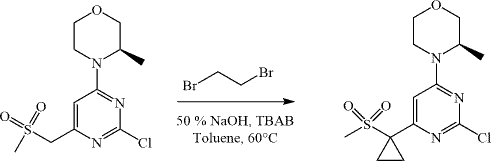

A mixture of (3R)-4-[2-chloro-6-(methanesulfonylmethyl)pyrimidin-4-yl]-3-methylmorpholine (125 mg, 0.41 mmol), 1,2-dibromoethane (154 mg, 0.82 mmol), NaOH (10.0 M in H₂O, 0.4 mL, 4.0 mmol) and TBAB (26 mg, 0.08 mmol) in Toluene (4 mL) was stirred at 60° C. for 3 h. LC-MS showed the reaction was complete. The reaction mixture was diluted with EA (40 mL), then washed with water and brine, dried over anhydrous Na₂SO₄, filtered and concentrated. The residue was purified by column chromatography on silica gel (PE : EA = 1:1, V/V) to afford the desired product (110 mg, yield: 81%). LC/MS( ESI): m/z 332 [M+H]⁺.

### Step 6. Tert-butyl (R)-5-((4-(3-methylmorpholino)-6-(1-(methylsulfonyl)cyclopropyl) pyrimidin-2-yl)amino)-1H-pyrazole-1-carboxylate (5-9)

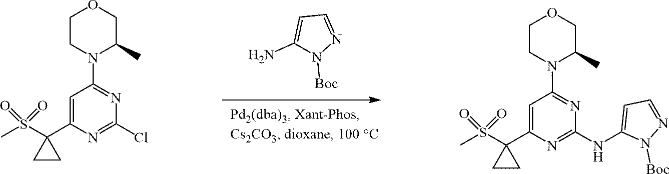

To a solution of (3R)-4-[2-chloro-6-(1-methanesulfonylcyclopropyl)pyrimidin-4-yl]-3-methylmorpholine (200 mg, 0.60 mmol) and tert-butyl 5-amino-1H-pyrazole-1-carboxylate (166 mg, 0.90 mmol) in Dioxane (10 mL) were added Pd₂(dba)₃ (55 mg, 0.06 mmol), Xant-Phos (34 mg, 0.06 mmol) and Cs₂CO₃ (394 mg, 1.21 mmol). The mixture was stirred at 100° C. for 6 h under N₂ atmosphere. LC-MS showed the reaction was complete. The reaction mixture was diluted with EA (40 mL), then washed with water and brine, dried over anhydrous Na₂SO₄, filtered and concentrated. The residue was purified by column chromatography on silica gel (PE : EA = 1:2, V/V) to afford the desired product (129 mg, yield: 44%). LC/MS( ESI): m/z 479 [M+H]⁺.

### Step 7. (R)-4-(3-methylmorpholino)-6-(1-(methylsulfonyl)cyclopropyl)-N-(1H-pyrazol-5-yl)pyrimidin-2-amine (5)

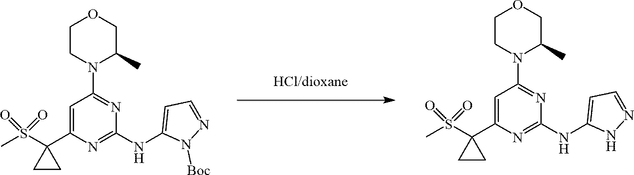

A mixture of tert-butyl5-{[4-(1-methanesulfonylcyclopropyl)-6-[(3R)-3-methyl morpholin-4-yl]pyrimidin-2-yl]amino}-1H-pyrazole-1-carboxylate (60 mg, 0.12 mmol) in HCl solution (4.0 M in dioxane, 3.0 mL) was stirred at room temperature for 10 h. LC-MS showed the reaction was complete. The reaction mixture was concentrated under reduced pressure. The residue was purified by Prep-HPLC (C₁₈, 10-95%, MeOH in H₂O with 0.1% HCOOH) to give the desired product (20 mg, yield: 42%). LC/MS (ESI): m/z 379 [M+H]⁺. ¹H NMR (400 MHz, DMSO) δ 12.29 (s, 1H), 9.51 (s, 1H),7.58 (s, 1H), 6.38 (s, 2H), 4.44 (s, 1H), 4.05 (d, J = 12.8 Hz, 1H), 3.94 (dd, J = 11.4, 3.4 Hz, 1H), 3.74 (d, J = 11.4 Hz, 1H), 3.59 (dd, J = 11.5, 2.9 Hz, 1H), 3.46 (s, 1H), 3.25 (s, 3H), 3.18 (s, 1H), 1.60 (t, J = 5.7 Hz, 2H), 1.50 (s, 2H), 1.21 (d, J = 6.7 Hz, 3H).

### Example 6

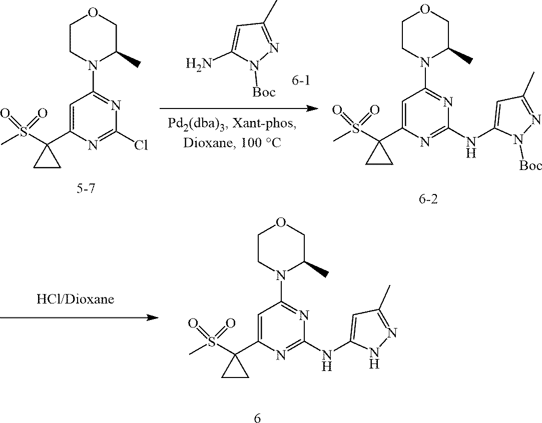

### Step 1. Tert-butyl 5-{[4-(1-methanesulfonylcyclopropyl)-6-[(3R)-3-methylmorpholin -4-yl]pyrimidin-2-yl]amino}-3-methyl-1H-pyrazole-1-carboxylate (6-2)

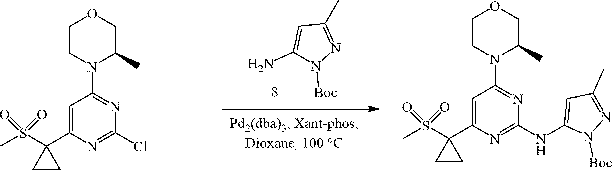

To a solution of (3R)-4-[2-chloro-6-(1-methanesulfonylcyclopropyl)pyrimidin-4-yl] -3-methylmorpholine (100 mg, 0.30 mmol) and tert-butyl 5-amino-3-methyl-1H -pyrazole-1-carboxylate (89.2 mg, 0.45 mmol) in Dioxane (5 mL) was added Cs₂CO₃ (196.4 mg, 0.60 mmol), Xant-Phos (17.4 mg, 0.03 mmol) and Pd₂(dba)₃ (24.4 mg, 0.03 mmol). The mixture was stirred at 100° C. for 6 h under nitrogen atmosphere.

LC-MS showed the reaction was complete. The reaction mixture was diluted with EA (40 mL), then washed with water and brine, dried over anhydrous Na₂SO₄, filtered and concentrated. The residue was purified by column chromatography on silica gel (PE : EA = 1:1, V/V) to afford the desired product (130 mg, yield: 87%). LC/MS (ESI): m/z 493 [M+H]⁺.

### Step 2. 4-(1-methanesulfonylcyclopropyl)-N-(3-methyl-1H-pyrazol-5-yl)-6-[(3R)-3-methylmorpholin-4-yl]pyrimidin-2-amine (6)

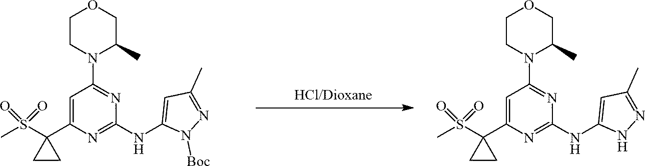

To a solution of tert-butyl 5-{[4-(1-methanesulfonylcyclopropyl)-6-[(3R)-3-methyl morpholin-4-yl]pyrimidin-2-yl]amino}-3-methyl-1H-pyrazole-1-carboxylate (120 mg, 0.24 mmol ) in DCM (2 mL) was added HCl solution (4 M in dioxane, 2 mL). The mixture was stirred at room temperature for 2 h. LC-MS showed the reaction was complete. The reaction mixture was concentrated under vacuo. The residue was purified by Prep-HPLC (C18, 10-95% MeCN in H₂O with 0.1% ammonia) to give the desired product (32.6 mg, yield: 34%). LC/MS (ESI) m/z: 393 [M+H]⁺. ¹H NMR (400 MHz, DMSO) δ 9.21 (s, 1H), 6.31 (s, 1H), 6.15 (s, 1H), 4.40 (s, 1H), 4.02 (d, J = 11.7 Hz, 1H), 3.93 (d, J = 8.1 Hz, 1H), 3.73 (d, J = 11.3 Hz, 1H), 3.58 (dd, J = 11.6, 2.9 Hz, 2H), 3.25 (s, 3H), 3.16 (d, J = 10.8 Hz, 1H), 2.19 (s, 3H), 1.58 (s, 2H), 1.47 (s, 2H), 1.20 (d, J = 6.7 Hz, 3H).

Compound 6 can be prepared using the following scheme:

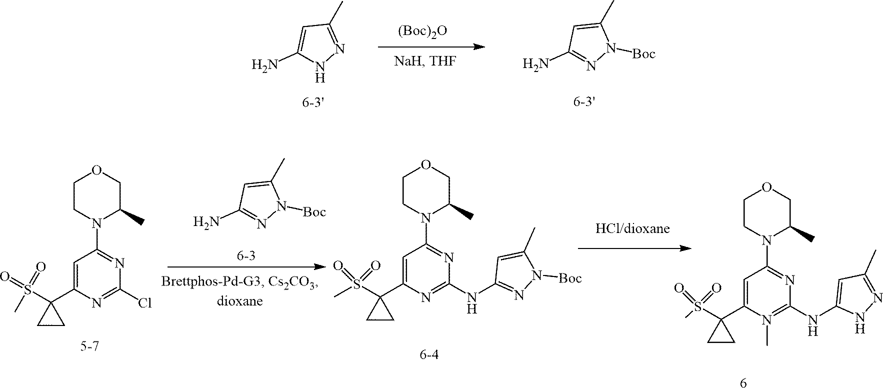

### Step 1. Tert-butyl 3-amino-5-methyl-1H-pyrazole-1-carboxylate (6-3)

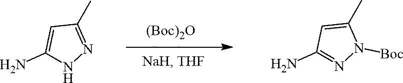

To a solution of 3-methyl-1H-pyrazol-5-amine (25 g, 257.41 mmol) in THF (800 mL) at 0° C. was added NaH (60%, 10.81 g, 270.28 mmol) portion wise. After stirring at 0° C. for 30 min, (Boc)₂O (58.99 g, 270.28 mmol) was added in one portion. The mixture was stirred at room temperature for 1 h. TLC showed the reaction was complete. The reaction mixture was poured into saturated NH₄Cl aqueous solution and extracted with DCM (600 mL×2) twice. The combined organic layer was separated, then washed with brine, dried over anhydrous Na₂SO₄, filtered and concentrated. The residue was purified by column chromatography on silica gel (PE : EA = 2:1, V/V) to give the desired product (19 g, yield: 37.42%). ¹HNMR (400 MHz, CDCl₃) δ 5.59 (d,J = 0.9 Hz, 1H), 3.89 (s, 2H), 2.44 (d,J = 0.9 Hz, 3H), 1.62 (s, 9H).

### Step 2. (R)-tert-butyl 5-methyl-3-((4-(3-methylmorpholino)-6-(1-(methyl sulfonyl)cyclopropyl)pyrimidin-2-yl)amino)-1H-pyrazole-1-carboxylate (6-4)

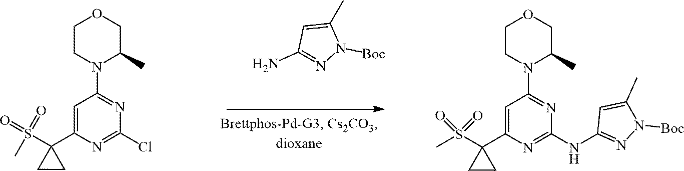

To a solution of (3R)-4-[2-chloro-6-(1-methanesulfonylcyclopropyl)pyrimidin-4-yl]-3-methylmorpholine (15.0 g, 45.20 mmol) and tert-butyl 3-amino-5-methyl-1H-pyrazole-1-carboxylate (10.7 g, 54.24 mmol) in Dioxane (600 mL) were added BrettPhos-Pd-G3 (906 mg, 4.41 mmol) and Cs₂CO₃ (29.45 g, 90.4 mmol). The mixture was stirred at 100° C. overnight under N₂ atmosphere. The reaction mixture was diluted with EA (1.0 L), then washed with water and brine, dried over anhydrous Na₂SO₄, filtered and concentrated. The residue was purified by column chromatography on silica gel (DCM : MeOH = 20 : 1, V/V) to afford the desired product (17 g, yield: 76%). LC/MS (ESI): m/z 493 [M+H]⁺.

### Step 3. (R)-N-(3-methyl-1H-pyrazol-5-yl)-4-(3-methylmorpholino)-6-(1-(methylsulfonyl)cyclopropyl)pyrimidin-2-amine (6)

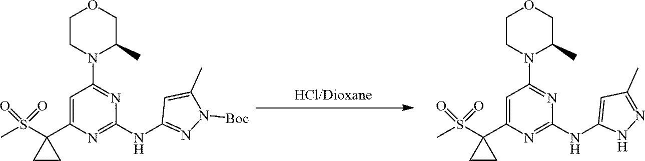

A mixture of (R)-tert-butyl 5-methyl-3-((4-(3-methylmorpholino)-6-(1-(methyl sulfonyl)cyclopropyl)pyrimidin-2-yl)amino)-1H-pyrazole-1-carboxylate (17.0 g, 34.51 mmol) in HCl solution (4.0 M in dioxane, 100.0 mL) was stirred at room temperature for 12h. The reaction mixture was concentrated under reduced pressure to dryness, the residue was diluted with EA (200 mL) and saturated NaHCO₃ aqueous solution (200 mL). The resulting mixture was stirred at room temperature overnight. The organic layer was separated, then washed with brine, dried over anhydrous Na₂SO₄, filtered and concentrated. The residue was purified by Prep-HPLC (C₁₈, 10-95%, MeOH in H₂O with 0.1% HCOOH) to give the desired product (10 g, yield: 73%). LC/MS (ESI): m/z 393 [M+H]⁺. ¹H NMR (400 MHz, DMSO) δ 11.78 (s, 1H), 9.10 (s, 1H), 6.23 (d, J = 29.9 Hz, 2H), 4.38 (s, 1H), 4.07 - 3.87 (m, 2H), 3.73 (d, J = 11.4 Hz, 1H), 3.58 (dd, J = 11.5, 2.9 Hz, 1H), 3.43 (td, J = 11.8, 2.9 Hz, 1H), 3.26 (s, 3H), 3.13 (td, J = 12.9, 3.7 Hz, 1H), 2.19 (s, 3H), 1.19 (d, J = 6.7 Hz, 3H).

### Example 7

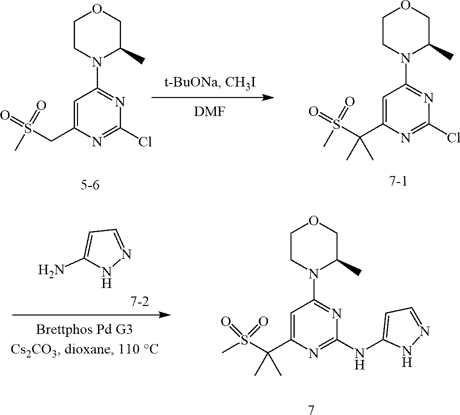

### Step 1. (R)-4-(2-chloro-6-(2-(methylsulfonyl)propan-2-yl)pyrimidin-4-yl)-3-methyl morpholine (7-1)

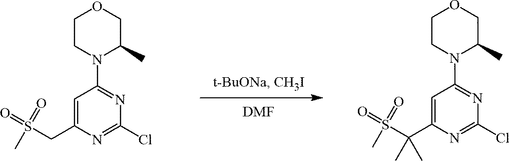

To a solution of (3R)-4-[2-chloro-6-(methanesulfonylmethyl)pyrimidin-4-yl]-3-methylmorpholine (900 mg, 2.94 mmol) and t-BuONa (849 mg, 8.82 mmol) in anhydrous DMF (16 mL) at 0° C. was added a solution of CH₃I (1.26 g, 8.85 mmol) in anhydrous DMF (1 mL) dropwise. After the addition, the resulting mixture was stirred at room temperature for 3 h. LC-MS showed the reaction was complete. The reaction mixture was diluted with EA (40 mL), then washed with water and brine, dried over anhydrous Na₂SO₄, filtered and concentrated. The residue was purified by column chromatography on silica gel (PE : EA = 10:1, V/V) to afford the desired product (870 mg, yield: 88%). LC/MS( ESI): m/z 334 [M+H]⁺.

### Step 2. (R)-4-(3-methylmorpholino)-6-(2-(methylsulfonyl)propan-2-yl)-N-(1H-pyrazol-5-yl)pyrimidin-2-amine (7)

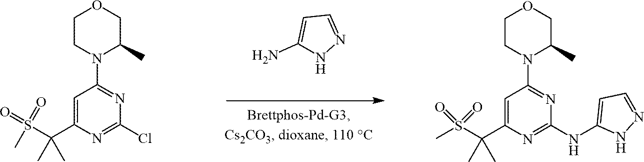

A mixture of (3R)-4-[2-chloro-6-(2-methanesulfonylpropan-2-yl)pyrimidin-4-yl]-3-methylmorpholine (100 mg, 0.30 mmol), 1H-pyrazol-5-amine (37 mg, 0.44 mmol), BrettPhos Pd G3 (27 mg, 0.03 mmol) and Cs₂CO₃ (293 mg, 0.90 mmol) in Dioxane (5 mL) was stirred at 110° C. for 10 h under N₂ atmosphere. LC-MS showed the reaction was complete. The reaction mixture was diluted with EA (40 mL), then washed with water and brine, dried over anhydrous Na₂SO₄, filtered and concentrated under vacuo. The residue was purified by Prep-HPLC (C₁₈, 10-95%, MeOH in H₂O with 0.1% HCOOH) to give the desired product (36.7 mg, yield: 32%). LC/MS (ESI): m/z 381 [M+H]⁺. ¹H NMR (400 MHz, DMSO) δ 12.18 (s, 1H), 9.26 (s, 1H), 7.52 (s, 1H), 6.41 (s, 1H), 6.30 (s, 1H), 4.42 (s, 1H), 4.03 (d, J = 12.9 Hz, 1H), 3.94 (dd, J = 11.4, 3.3 Hz, 1H), 3.73 (d, J = 11.4 Hz, 1H), 3.59 (dd, J = 11.5, 3.0 Hz, 1H), 3.44 (dd, J = 11.8, 9.0 Hz, 1H), 3.14 (td, J = 12.9, 3.7 Hz, 1H), 3.01 (s, 3H), 1.67 (s, 6H), 1.19 (d, J = 6.7 Hz, 3H).

### Example 8

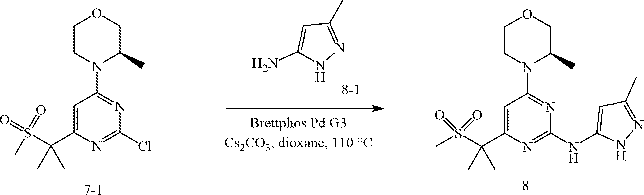

### Step 1. (R)-N-(3-methyl-1H-pyrazol-5-yl)-4-(3-methylmorpholino)-6-(2-(methylsulfonyl)propan-2-yl)pyrimidin-2-amine (8)

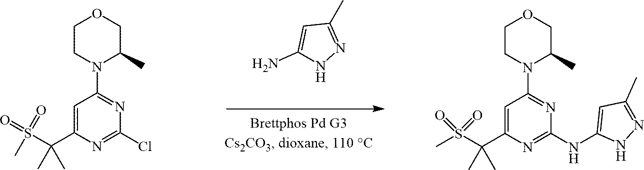

A mixture of (3R)-4-[6-chloro-4-(2-methanesulfonylpropan-2-yl)pyridin-2-yl]-3-methylmorpholine (100 mg, 0.30 mmol), 3-methyl-1H-pyrazol-5-amine (58 mg, 0.60 mmol), BrettPhos Pd G3 (27 mg, 0.03 mmol) and Cs₂CO₃ (293 mg, 0.90 mmol) in dioxane (4 mL) was stirred at 110° C. for 10 h under N₂ atmosphere. LC-MS showed the reaction was complete. The reaction mixture was diluted with EA (40 mL), then washed with water and brine, dried over anhydrous Na₂SO₄, filtered and concentrated under vacuo. The residue was purified by Prep-HPLC (C₁₈, 10-95%, MeOH in H₂O with 0.1% HCOOH) to give the desired product as a white solid (44.8 mg, yield: 37%). LC/MS (ESI): m/z 395 [M+H]⁺. ¹HNMR (400 MHz, DMSO) δ 11.98 (s, 1H), 9.08 (s, 1H), 8.13 (s, 1H), 6.28 (s, 1H), 6.16 (s, 1H), 4.40 (s, 1H), 4.02 (d, J = 13.0 Hz, 1H), 3.93 (dd, J = 11.4, 3.4 Hz, 1H), 3.73 (d, J = 11.4 Hz, 1H), 3.59 (dd, J = 11.5, 2.9 Hz, 1H), 3.44 (td, J = 11.8, 2.8 Hz, 1H), 3.13 (td, J = 13.0, 3.7 Hz, 1H), 3.01 (s, 3H), 2.18 (s, 3H), 1.66 (s, 6H), 1.19 (d, J = 6.7 Hz, 3H).

### Example 9

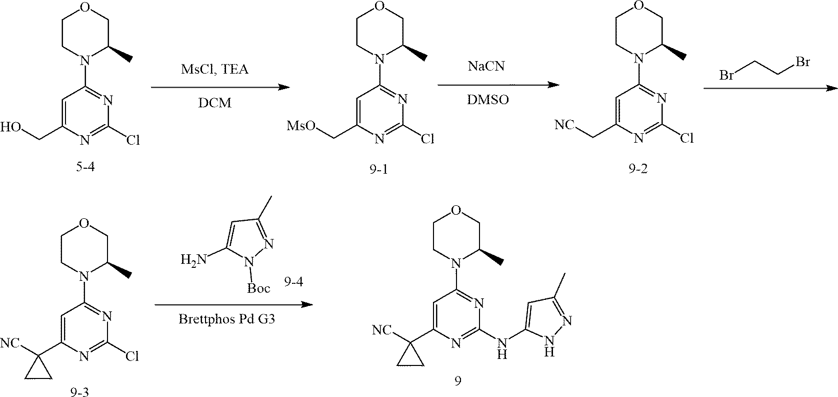

### Step 1. (R)-(2-chloro-6-(3-methylmorpholino)pyrimidin-4-yl)methylmethane sulfonate (9-1)

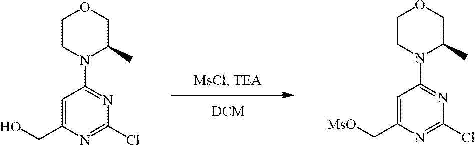

To a solution of (R)-(2-chloro-6-(3-methylmorpholino)pyrimidin-4-yl)methanol (1 g, 4.10 mmol) and TEA (623 mg, 6.15 mmol) in DCM (30 mL) at 0° C. was added a solution of MsCl (564 mg, 4.92 mmol) in DCM (2 mL) dropwise. The resulting mixture was stirred at room temperature for 3h. LC-MS showed the reaction was complete. The reaction mixture was diluted with EA (40 mL), then washed with water and brine, dried over anhydrous Na₂SO₄, filtered and concentrated. The residue was purified by column chromatography on silica gel (PE : EA = 10:1, V/V) to afford the desired product (1.06 mg, yield: 80%). LC/MS( ESI): m/z 322 [M+H]⁺.

### Step 2. (R)-2-(2-chloro-6-(3-methylmorpholino)pyrimidin-4-yl)acetonitrile (9-2)

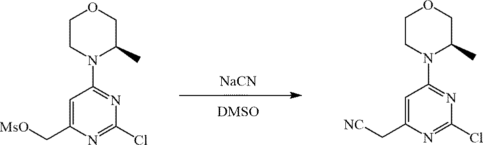

To a solution of NaCN (184 mg, 3.75 mmol) in DMSO (20 mL) was added a solution of (R)-(2-chloro-6-(3-methylmorpholino)pyrimidin-4-yl)methyl methanesulfonate (1 g, 3.10 mmol) dropwise. The resulting mixture was stirred at room temperature for 1h. LC-MS showed the reaction was complete. The reaction mixture was diluted with EA (40 mL), then washed with ice-water and brine, dried over anhydrous Na₂SO₄, filtered and concentrated The residue was purified by column chromatography on silica gel (PE : EA = 3:1, V/V) to afford the desired product (300 mg, yield: 38%). LC/MS( ESI): m/z 253 [M+H]⁺.

### Step 3. (R)-1-(2-chloro-6-(3-methylmorpholino)pyrimidin-4-yl)cyclopropane carbonitrile (9-3)

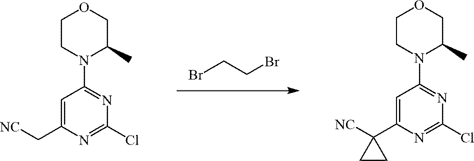

To a solution of (R)-2-(2-chloro-6-(3-methylmorpholino)pyrimidin-4-yl)acetonitrile (100 mg, 0.40 mmol), 1,2-dibromoethane (338 mg, 1.79 mmol) and TBAB (32.2 mg, 0.1 mmol) in 2-MeTHF (15 mL) was added a solution of KOH (1.57 g, 28.0 mmol) in H₂O (15 mL). The resulting mixture was stirred at room temperature for 12 h. LC-MS showed the reaction was complete. The reaction mixture was diluted with EA (50 mL), then washed with water and brine, dried over anhydrous Na₂SO₄, filtered and concentrated. The residue was purified by column chromatography on silica gel (PE : EA = 5:1, V/V) to afford the desired product (50 mg, yield: 46%). LC/MS( ESI): m/z 279 [M+H]⁺.

### Step 4. (R)-1-(2-((3-methyl-1H-pyrazol-5-yl)amino)-6-(3-methylmorpholino)pyrimidin-4-yl)cyclopropanecarbonitrile (9)

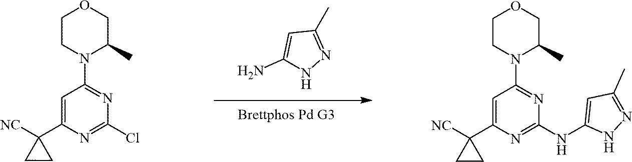

To a solution of (R)-1-(2-chloro-6-(3-methylmorpholino)pyrimidin-4-yl)cyclo propanecarbonitrile (50 mg, 0.18 mmol), 3-methyl-1H-pyrazol-5-amine (35 mg, 0.36 mmol) and Cs₂CO₃ (117.3 mg, 0.36 mmol) in Dioxane (5 mL) was added BrettPhos Pd G3 (16 mg, 0.018 mmol). The mixture was stirred at 100° C. for 16 h. LC-MS showed the reaction was complete. The reaction mixture was diluted with EA (40 mL), then washed with water and brine, dried over anhydrous Na₂SO₄, filtered and concentrated under vacuo. The residue was purified by Prep-HPLC (C₁₈, 10-95%, MeOH in H₂O with 0.1% HCOOH) to give the desired product (8.2 mg, yield: 13%). LC/MS (ESI): m/z 340 [M+H]⁺. ¹H NMR (400 MHz, DMSO) δ 8.97 (s, 1H), 6.17 (s, 2H), 4.35 (s, 1H), 4.04 - 3.87 (m, 2H), 3.72 (d, J = 11.4 Hz, 1H), 3.58 (dd, J = 11.5, 2.9 Hz, 1H), 3.47 - 3.39 (m, 4H), 3.13 (td, J = 12.9, 3.7 Hz, 2H), 2.17 (s, 3H), 1.70 (s, 4H), 1.19 (d, J = 6.7 Hz, 3H).

### Example 10

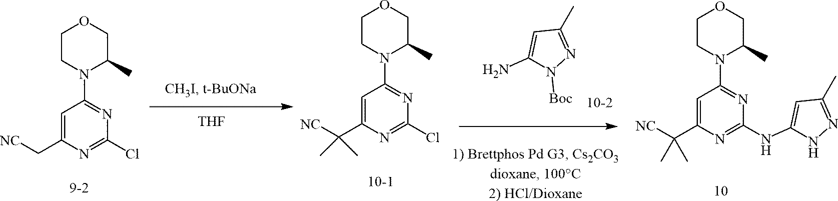

### Step 1. (R)-2-(2-chloro-6-(3-methylmorpholino)pyrimidin-4-yl)-2-methyl propanenitrile (10-1)

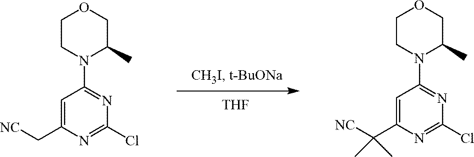

To a solution of (R)-(2-chloro-6-(3-methylmorpholino)pyrimidin-4-yl)methyl methanesulfonate (360 mg, 1.42 mmol) and t-BuONa (274 mg, 2.85 mmol) in anhydrous THF (15 mL) at 0° C. was added a solution of CH₃I (605 mg, 4.26 mmol) in anhydrous THF (1 mL) dropwise. After the addition, the resulting mixture was stirred at room temperature for 12 h. LC-MS showed the reaction was complete. The reaction mixture was diluted with EA (40 mL), then washed with water and brine, dried over anhydrous Na₂SO₄, filtered and concentrated. The residue was purified by column chromatography on silica gel (PE : EA = 5:1, V/V) to afford the desired product (300 mg, yield: 75%). LC/MS( ESI): m/z 281 [M+H]⁺.

### Step 2. (R)-2-methyl-2-(2-((3-methyl-1H-pyrazol-5-yl)amino)-6-(3-methylmorpho-lino)pyrimidin-4-yl)propanenitrile (10)

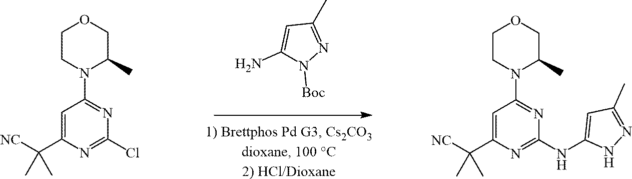

To a solution of (R)-2-(2-chloro-6-(3-methylmorpholino)pyrimidin-4-yl)-2-methyl propanenitrile (50 mg, 0.18 mmol), tert-butyl 5-amino-3-methyl-1H-pyrazole-1-carboxylate (70 mg, 0.36 mmol) and Cs₂CO₃ (174 mg, 0.53 mmol) in Dioxane (5 mL) was added BrettPhos Pd G3 (16 mg, 0.018 mmol). The mixture was stirred at 100° C. for 16 h. LC-MS showed the reaction was complete. The reaction mixture was diluted with EA (40 mL), then washed with water and brine, dried over anhydrous Na₂SO₄, filtered and concentrated under vacuo. The residue was dissolved in DCM (4 mL), then HCl solution (4 M in dioxane, 2 mL) was added. The mixture was stirred at room temperature for 2 h. LC-MS showed the reaction was complete. The reaction mixture was concentrated under vacuo. The residue was purified by Prep-HPLC (C₁₈, 10-95%, MeOH in H₂O with 0.1% HCOOH) to give the desired product (8 mg, yield: 13%). LC/MS (ESI): m/z 342 [M+H]⁺. ¹H NMR (400 MHz, DMSO) δ 11.78 (s, 1H), 9.07 (s, 1H), 6.26 (d, J = 17.8 Hz, 2H), 4.40 (dd, J = 13.5, 7.2 Hz, 1H), 4.01 (d, J = 13.2 Hz, 1H), 3.93 (dd, J = 11.3, 3.3 Hz, 1H), 3.72 (d, J = 11.4 Hz, 1H), 3.58 (dd, J = 11.4, 2.9 Hz, 1H), 3.45 - 3.42 (m, 1H), 3.17 - 3.10 (m, 1H), 2.17 (s, 3H), 1.64 (s, 6H), 1.19 (d, J = 6.7 Hz, 3H).

### Example 11

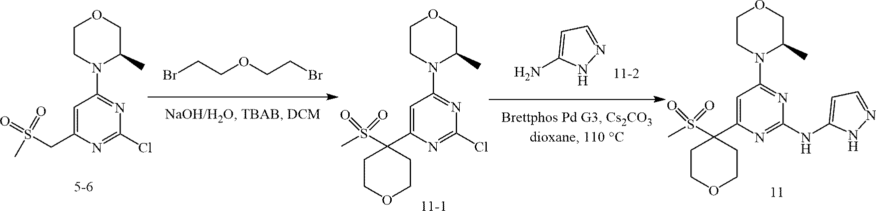

### Step 1. (R)-4-(2-chloro-6-(4-(methylsulfonyl)tetrahydro-2H-pyran-4-yl)pyrimidin-4-yl)-3-methylmorpholine (11-1)

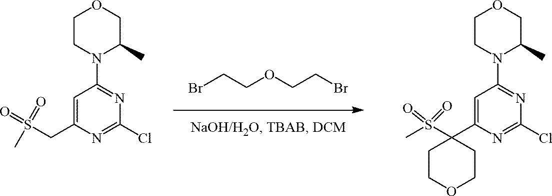

A mixture of (3R)-4-[2-chloro-6-(methanesulfonylmethyl)pyrimidin-4-yl]-3-methyl morpholine (400 mg, 1.31 mmol), 1-bromo-2-(2-bromoethoxy)ethane (905 mg, 3.93 mmol), TBAB (42 mg, 0.13 mmol) and NaOH (100 M in H₂O, 1.31 mL, 13.1 mmol) in DCM (20 mL) was stirred at room temperature for 24 h. LC-MS showed the reaction was complete. The reaction mixture was diluted with DCM (40 mL), then washed with water and brine, dried over anhydrous Na₂SO₄, filtered and concentrated under vacuo. The residue was purified by column chromatography on silica gel (PE: EA = 3:1, V/V) to afford the desired product (147 mg, yield: 30%). LC/MS (ESI): m/z 376 [M+H]⁺.

### Step 2. (R)-4-(3-methylmorpholino)-6-(4-(methylsulfonyl)tetrahydro-2H-pyran-4-yl)-N-(1H-pyrazol-5-yl)pyrimidin-2-amine (11)

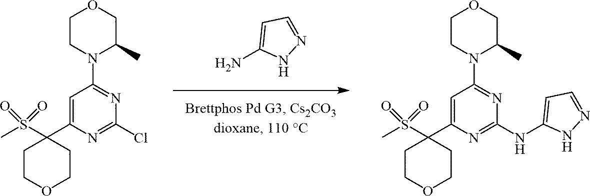

A mixture of (3R)-4-[2-chloro-6-(4-methanesulfonyloxan-4-yl)pyrimidin-4-yl]-3-methylmorpholine (70 mg, 0.19 mmol), 1H-pyrazol-5-amine (31 mg, 0.37 mmol), BrettPhos Pd G3 (17 mg, 0.02 mmol) and Cs₂CO₃ (182 mg, 0.56 mmol) in Dioxane (3 mL) was stirred at 110° C. for 10 h under N₂ atmosphere. LC-MS showed the reaction was complete. The reaction mixture was diluted with EA (40 mL), then washed with water and brine, dried over anhydrous Na₂SO₄, filtered and concentrated under vacuo. The residue was purified by Prep-HPLC (C₁₈, 10-95%, MeOH in H₂O with 0.1% HCOOH) to give the desired product (40 mg, yield: 50%). LC/MS (ESI): m/z 423 [M+H]⁺. ¹H NMR (400 MHz, DMSO) δ 12.09 (s, 1H), 9.19 (s, 1H), 7.53 (s, 1H), 6.40 (s, 2H), 4.41 (d, J = 4.6 Hz, 1H), 4.08 (d, J = 12.8 Hz, 1H), 3.91 (ddd, J = 16.0, 10.8, 4.0 Hz, 3H), 3.73 (d, J = 11.5 Hz, 1H), 3.61 (dd, J = 11.5, 2.9 Hz, 1H), 3.46 (td, J = 11.9, 2.9 Hz, 1H), 3.17 (ddd, J = 19.1, 16.3, 8.1 Hz, 3H), 2.85 (s, 3H), 2.64 (d, J = 13.1 Hz, 2H), 2.13 (t, J = 11.8 Hz, 2H), 1.19 (d, J = 6.7 Hz, 3H).

### Example 12

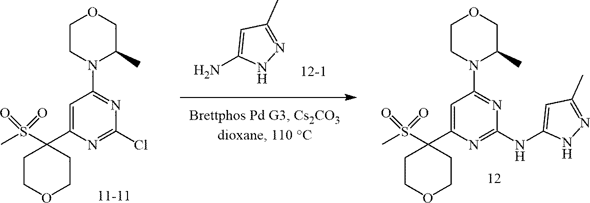

### Step 1. (R)-N-(3-methyl-1H-pyrazol-5-yl)-4-(3-methylmorpholino)-6-(4-(methylsulfonyl)tetrahydro-2H-pyran-4-yl)pyrimidin-2-amine (12)

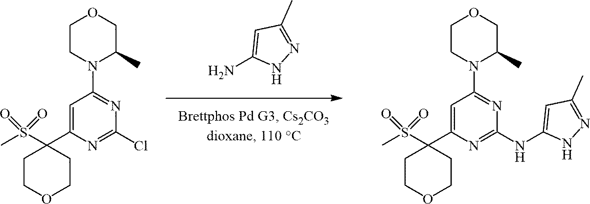

A mixture of (3R)-4-[2-chloro-6-(4-methanesulfonyloxan-4-yl)pyrimidin-4-yl]-3-methylmorpholine (70 mg, 0.18 mmol), 3-methyl-1H-pyrazol-5-amine (36 mg, 0.37 mmol), BrettPhos Pd G3 (17 mg, 0.02 mmol) and Cs₂CO₃ (182 mg, 0.56 mmol) in Dioxane (3 mL) was stirred at 110° C. for 10 h under N₂ atmosphere. LC-MS showed the reaction was complete. The reaction mixture was diluted with EA (40 mL), then washed with water and brine, dried over anhydrous Na₂SO₄, filtered and concentrated under vacuo. The residue was purified by Prep-HPLC (C₁₈, 10-95%, MeOH in H₂O with 0.1% HCOOH) to give the desired product (48 mg, yield: 61%). LC/MS (ESI): m/z 437 [M+H]⁺. ¹H NMR (400 MHz, DMSO) δ 11.78 (s, 1H), 903 (s, 1H), 8.16 (s, 1H), 6.38 (s, 1H), 6.16 (s, 1H), 4.39 (s, 1H), 4.08 (d, J = 13.0 Hz, 1H), 3.98 - 3.84 (m, 3H), 3.72 (d, J = 11.4 Hz, 1H), 3.61 (dd, J = 11.4, 2.9 Hz, 1H), 3.46 (td, J = 11.9, 2.8 Hz, 1H), 3.16 (ddd, J = 19.2, 16.3, 8.1 Hz, 3H), 2.85 (s, 3H), 2.63 (d, J = 13.0 Hz, 2H), 2.24 - 2.02 (m, 5H), 1.19 (d, J = 6.7 Hz, 3H).

### Example 13

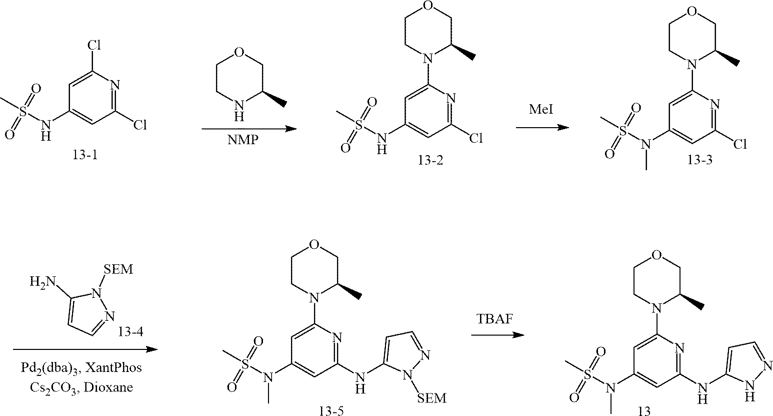

### Step 1. (R)-N-(2-chloro-6-(3-methylmorpholino)pyridin-4-yl)methanesulfonamide (13-2)

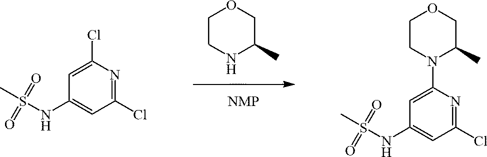

To a solution of N-(2,6-dichloropyridin-4-yl)methanesulfonamide (500 mg, 2.07 mmol) in NMP (15 mL) was added (3R)-3-methylmorpholine (629 mg, 6.22 mmol). The mixture was stirred at 170° C. for 1 h under microwave irradiation. LC-MS showed the reaction was complete. The mixture was diluted with water (60 mL) and extracted with EA (30 mL×3) thrice. The combined organic phase was washed with brine, dried over Na₂SO₄, filtered and concentrated to dryness. The residue was purified by column chromatography on silica gel (PE : EA = 1:1, V/V) to give the desired product (425 mg, yield: 67.02%). LC/MS (ESI): m/z 306 [M+H]⁺.

### Step 2. (R)-N-(2-chloro-6-(3-methylmorpholino)pyridin-4-yl)-N-methylmethane sulfonamide (13-3)

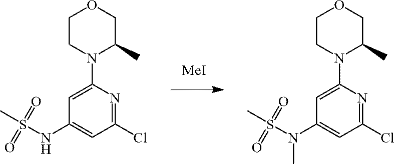

To a mixture of (R)-N-(2-chloro-6-(3-methylmorpholino)pyridin-4-yl)methane sulfonamide (250 mg, 0.82 mmol) and K₂CO₃ (339 mg, 2.45 mmol) in DMF (8 mL) was added MeI (174 mg, 1.23 mmol). The mixture was stirred at room temperature for 2 h. LC-MS showed the reaction was complete. The reaction mixture was poured into H₂O (30 mL) and extracted with EA (30 mL×3) thrice. The combined organic phase was washed with brine, dried over anhydrous Na₂SO₄, filtered and concentrated to dryness. The residue was purified by column chromatography on silica gel (PE : EA = 1:1, V/V) to give the desired product (218 mg, yield: 83.4%). LC/MS (ESI): m/z 320 [M+H]⁺.

### Step 3. (R)-N-methyl-N-(2-(3-methylmorpholino)-6-((1-((2-(trimethylsilyl)ethoxy) methyl)-1H-pyrazol-5-yl)amino)pyridin-4-yl)methanesulfonamide (13-5)

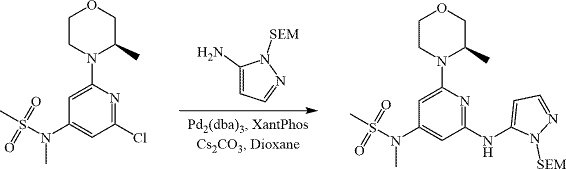

To a solution of (R)-N-(2-chloro-6-(3-methylmorpholino)pyridin-4-yl)-N-methyl methanesulfonamide (100 mg, 0.31 mmol), 1-({[2-(trimethylsilyl)ethoxy]methyl}-λ^2-chloranyl)-1H-pyrazol-5-amine (100 mg, 0.47 mmol) and Cs₂CO₃ (306 mg, 0.94 mmol) in dioxane (5 mL) were added Pd₂(dba)₃ (29 mg, 0.031 mmol) and XantPhos (36 mg, 0.06 mmol). The mixture was stirred at 100° C. for 16 h under N₂ atmosphere. LC-MS showed the reaction was complete. The reaction mixture was diluted with EA (60 mL), then washed with water and brine, dried over anhydrous Na₂SO₄, filtered and concentrated to dryness. The residue was purified by column chromatography on silica gel (PE : EA = 1:1, V/V) to give the desired product (118 mg, yield: 76%). LC/MS (ESI): m/z 497 [M+H]⁺.

### Step 4. (R)-N-(2-((1H-pyrazol-5-yl)amino)-6-(3-methylmorpholino)pyridin-4-yl)-N-methylmethanesulfonamide (13)

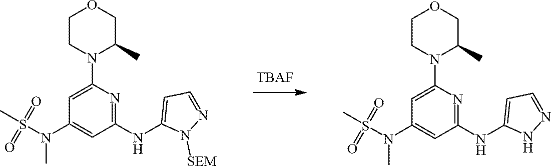 [SMILES: C[C@@H]1COCCN1c1cc(N(C)S(C)(=O)=O)cc(Nc2ccnn2COCC[Si](C)(C)C)n1 ; "TBAF" ; C[C@@H]1COCCN1c1cc(N(C)S(C)(=O)=O)cc(Nc2ccn[nH]2)n1]

A mixture of (R)-N-methyl-N-(2-(3-methylmorpholino)-6-((1-((2-(trimethylsilyl) ethoxy)methyl)-1H-pyrazol-5-yl)amino)pyridin-4-yl)methanesulfonamide (118 mg, 0.24 mmol) in TBAF solution (1 M in THF, 2 mL) was stirred at 60° C. for 2 h. LC-MS showed the reaction was complete. The mixture was diluted with H₂O and extracted with EA (30 mL×3) thrice. The combined organic phase was washed brine, dried over anhydrous Na₂SO₄, filtered and concentrated to dryness. The residue was purified by Prep-HPLC (C₁₈, 10-95%, MeOH in H₂O with 0.1% HCOOH) to give the desired product (20 mg, yield: 23%). LC/MS (ESI): m/z 367 [M+H]⁺. ¹H NMR (400 MHz, DMSO) δ 8.96 (s, 1H), 7.53 (d, J = 2.2 Hz, 1H), 6.47 (s, 1H), 6.30 (d, J = 2.1 Hz, 1H), 6.05 (d, J = 1.2 Hz, 1H), 4.29 - 4.23 (m, 1H), 3.93 (dd, J = 11.2, 3.2 Hz, 1H), 3.77 - 3.71 (m, 2H), 3.64 - 3.61 (m, 1H), 3.50 - 3.49 (m, 1H), 3.19 (s, 3H), 3.07 (dd, J = 12.6, 3.7 Hz, 1H), 3.01 (s, 3H), 1.13 (d, J = 6.6 Hz, 3H).

### Example 14

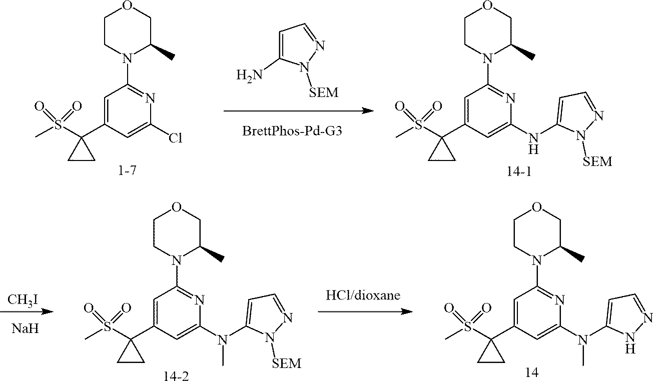

### Step 1. (R)-6-(3-methylmorpholino)-4-(1-(methylsulfonyl)cyclopropyl)-N-(1-((2-(tri-methylsilyl)ethoxy)methyl)-1H-pyrazol-5-yl)pyridin-2-amine (14-1)

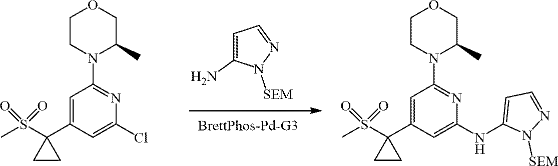

To a solution of (R)-4-(6-chloro-4-(1-(methylsulfonyl)cyclopropyl)pyridin-2-yl)-3- methylmorpholine (356.0 mg, 1.08 mmol) and 1-({[2-(trimethylsilyl)ethoxy]methyl }-ÀA2-chloranyl)-1H-pyrazol-5-amine (343.8 mg, 1.61 mmol) in dioxane (15 mL) were added Pd₂(dba)₃ (98.5 mg, 0.11 mmol), BrettPhos-Pd-G3 (13.7 mg, 0.015 mmol) and Cs₂CO₃ (701.2 mg, 2.15 mmol). The mixture was stirred at 100° C. overnight under nitrogen atmosphere. LC-MS showed the reaction was complete. The reaction was diluted with EA (40 mL), then washed with water and brine, dried over anhydrous Na₂SO₄, filtered and concentrated. The residue was purified by column chromatography on silica gel (PE : EA = 3:1, V/V) to afford the desired product (490 mg, yield: 89%). LC/MS (ESI) m/z: 508 [M+H]⁺.

### Step 2. (R)-N-methyl-6-(3-methylmorpholino)-4-(1-(methylsulfonyl)cyclopropyl)-N-(1-((2-(trimethylsilyl)ethoxy)methyl)-1H-pyrazol-5-yl)pyridin-2-amine (14-2)

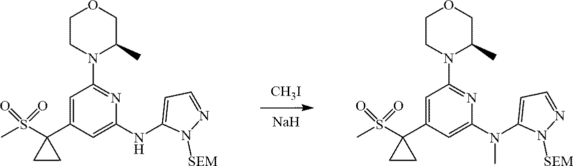

To solution of (R)-6-(3-methylmorpholino)-4-(1-(methylsulfonyl)cyclopropyl)-N- (1-((2-(trimethylsilyl)ethoxy)methyl)-1H-pyrazol-5-yl)pyridin-2-amine (200 mg, 0.39 mmol) in THF (5 mL) at 0° C. was added NaH (60%, 14.2 mg, 0.59 mmol) portion wise. The mixture was stirred at 0° C. for 30 min, then a solution of CH₃I (84.0 mg, 0.59 mmol) in THF (1 mL) was added drop wise. The resulting mixture was stirred at room temperature for an additional 1 h. LC-MS showed the reaction was complete. The reaction mixture was quenched with saturated NH₄Cl aqueous solution and extracted with EA (30 mL×2). The combined organic layer was washed with brine, dried over anhydrous Na₂SO₄, filtered and concentrated. The residue was purified by column chromatography on silica gel (DCM: MeOH = 50:1, V/V) to give the desired product (110 mg, yield: 53%). LC/MS (ESI) (m/z): 522 [M+H]⁺.

### Step 3. (R)-N-methyl-6-(3-methylmorpholino)-4-(1-(methylsulfonyl)cyclopropyl)-N -(1-((2-(trimethylsilyl)ethoxy)methyl)-1H-pyrazol-5-yl)pyridin-2-amine (14)

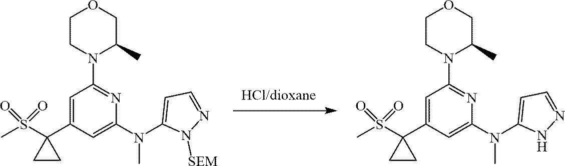 [SMILES: C[C@@H]1COCCN1c1cc(C2(S(C)(=O)=O)CC2)cc(N(C)c2ccnn2COCC[Si](C)(C)C)n1 ; "HCl/dioxane" ; C[C@@H]1COCCN1c1cc(C2(S(C)(=O)=O)CC2)cc(N(C)c2ccn[nH]2)n1]

A mixture of (R)-N-methyl-6-(3-methylmorpholino)-4-(1-(methylsulfonyl)cyclopro- pyl)-N-(1-((2-(trimethylsilyl)ethoxy)methyl)-1H-pyrazol-5-yl)pyridin-2-amine (110 mg, 0.21 mmol) in HCl solution (4 M in dioxane, 2 mL) was stirred at room temperature for 2 h. LC-MS showed the reaction was complete. The reaction mixture was concentrated under vacuo. The residue was purified by Pre-HPLC (C18, 10-95%, MeCN in H₂O with 0.1% HCOOH) to afford the desired product (14 mg, yield: 17%). LC/MS (ESI) m/z: 392 [M+H]⁺. ¹H NMR (400 MHz, DMSO) δ 12.40 (s, 1H), 8.37 (s, 1H), 7.66 (s, 1H), 6.48 (s, 1H), 6.24 (s, 1H), 6.21 (d, J = 1.9 Hz, 1H), 4.27 (d, J = 4.9 Hz, 1H), 3.97 - 3.80 (m, 3H), 3.72 (d, J = 11.2 Hz, 1H), 3.62 (dd, J = 11.3, 2.8 Hz, 2H), 3.53 - 3.42 (m, 3H), 3.05 (td, J = 12.6, 3.6 Hz, 2H), 2.91 (s, 4H), 1.53 (dd, J = 6.2, 4.1 Hz, 3H), 1.22 (t, J = 5.1 Hz, 3H), 1.14 (d, J = 6.6 Hz, 4H).

### Example 15

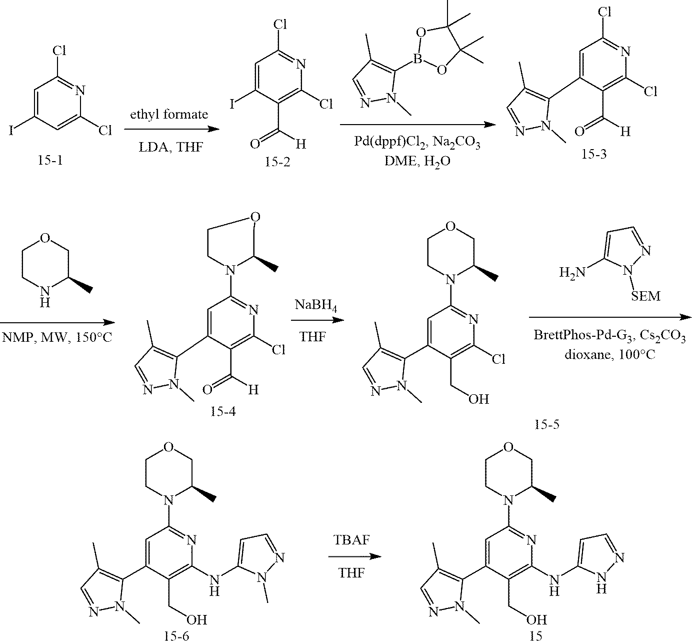

### Step 1. 2,6-dichloro-4-iodonicotinaldehyde (15-2)

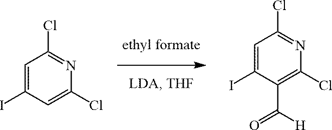

To solution of 2,6-dichloro-4-iodopyridine (1 g, 3.65 mmol) in THF (10 ml) at -78° C. was added LDA solution (2 M in THF, 2.74 mL, 5.48 mmol) drop wise. The mixture was stirred at -78° C. for 1 h, following by the addition of ethyl formate (0.44 mL, 5.48 mmol) in THF (1 ml) drop wise. The resulting mixture was stirred at -78° C. for an additional 2 h. LC-MS showed the reaction was complete. The reaction mixture was quenched with saturated NH₄Cl aqueous solution and extracted with EA (40 mL×2) twice. The combined organic layer was washed with brine, dried over anhydrous Na₂SO₄, filtered and concentrated. The residue was purified by column chromatography on silica gel (PE: EA= 30: 1, V/V) to give the desired product (553 mg, yield: 50%). LC/MS (ESI): m/z 302 [M+H]⁺.

### Step 2. 2,6-dichloro-4-(1,4-dimethyl-1H-pyrazol-5-yl)nicotinaldehyde (15-3)

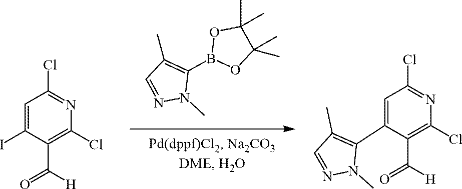

To a solution of 2,6-dichloro-4-iodopyridine-3-carbaldehyde (1.5 g, 4.97 mmol) and 1,4-dimethyl-5-(tetramethyl-1,3,2-dioxaborolan-2-yl)-1H-pyrazole (1.32 g, 5.96 mmol) in DME (90 mL) were added Pd(dppf)Cl₂ (360 mg, 0.50 mmol) and Na₂CO₃ (2.0 M in H₂O, 6 mL, 12.0 mmol). The mixture was charged with N₂ twice, then stirred at 100° C. overnight under N₂ atmosphere. LC-MS showed the reaction was complete. The reaction mixture was diluted with H₂O (100 mL) and extracted with EA (100 mLx2) twice. The combined organic layer was washed with brine, dried over anhydrous Na₂SO₄, filtered and concentrated. The residue was purified by column chromatography on silica gel (PE: EA= 10:1, V/V) to give the desired product (548 mg, yield: 41%). LC/MS (ESI): m/z 270 [M+H]⁺. ¹H NMR (400 MHz, DMSO) δ 9.98 (s, 1H), 7.79 (s, 1H), 7.37 (s, 1H), 3.59 (s, 3H), 1.79 (s, 3H).

### Step 3. (R)-2-chloro-4-(1,4-dimethyl-1H-pyrazol-5-yl)-6-(3-methylmorpholino) nicotinaldehyde (15-4)

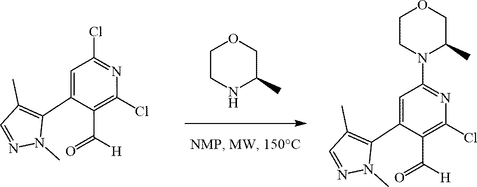

A solution of 2,6-dichloro-4-(1,4-dimethyl-1H-pyrazol-5-yl)pyridine-3-carbaldehyde (330 mg, 1.22 mmol) and (3R)-3-methylmorpholine (185 mg, 1.83 mmol) in NMP (14 mL) was stirred at 130° C. for 1 h under microwave irradiation. LC-MS showed the reaction was complete. The reaction mixture was quenched with H₂O (40 mL) and extracted with EA (50 mL×2) twice. The combined organic layer was washed with brine, dried over anhydrous Na₂SO₄, filtered and concentrated. The residue was purified by column chromatography on silica gel (PE: EA = 5:1, V/V) to give the desired product (142 mg, yield: 35%). LC/MS (ESI): m/z 335 [M+H]⁺.

### Step 4. (R)-(2-chloro-4-(1,4-dimethyl-1H-pyrazol-5-yl)-6-(3-methylmorpholino) pyridin-3-yl)methanol (15-5)

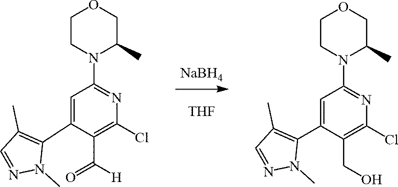 [SMILES: Cc1cnn(C)c1-c1cc(N2CCOC[C@H]2C)nc(Cl)c1C=O ; "NaBH4" ; Cc1cnn(C)c1-c1cc(N2CCOC[C@H]2C)nc(Cl)c1CO ; "THF"]

To a solution of 2-chloro-4-(1,4-dimethyl-1H-pyrazol-5-yl)-6-[(3R)-3-methyl morpholin-4-yl]pyridine-3-carbaldehyde (140 mg, 0.42 mmol) in THF (4 mL) was added NaBH₄ (14 mg, 0.42 mmol). The resulting mixture was stirred at 0° C. for 0.5 h. LC-MS showed the reaction was complete. The reaction mixture was quenched with H₂O and extracted with EA (40 mL×2) twice. The combined organic layer was washed with brine, dried over anhydrous Na₂SO₄, filtered and concentrated. The residue was purified by column chromatography on silica gel (PE: EA = 3:1, V/V) to give the desired product (140 mg, yield: 99%). LC/MS (ESI): m/z 337 [M+H]⁺.

### Step 5. (R)-(4-(1,4-dimethyl-1H-pyrazol-5-yl)-6-(3-methylmorpholino)-2-((1-((2-(trimethylsilyl)ethoxy)methyl)-1H-pyrazol-5-yl)amino)pyridin-3-yl)methanol (15-6)

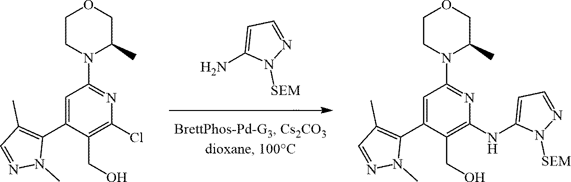

A mixture of [2-chloro-4-(1,4-dimethyl-1H-pyrazol-5-yl)-6-[(3R)-3-methylmorpholin-4-yl]pyridin-3-yl]methanol (135 mg, 0.40 mmol), 1-({[2-(trimethylsilyl)ethoxy] methyl}-λ^2-chloranyl)-1H-pyrazol-5-amine (128 mg, 0.60 mmol), BrettPhos-Pd-G3 (36 mg, 0.04 mmol) and Cs₂CO₃ (392 mg, 1.20 mmol) in dioxane (6 mL) was stirred at 100° C. overnight under nitrogen atmosphere. LC-MS showed the reaction was complete. The mixture was diluted with EA (60 mL), then washed with water and brine, dried over anhydrous Na₂SO₄, filtered and concentrated. The residue was purified by column chromatography on silica gel (PE: EA= 1:1, V/V) to give the desired product (46 mg, yield: 22%). LC/MS (ESI): m/z 514 [M+H]⁺.

### Step 6. (R)-(2-((1H-pyrazol-5-yl)amino)-4-(1,4-dimethyl-1H-pyrazol-5-yl)-6-(3-methylmorpholino)pyridin-3-yl)methanol (15)

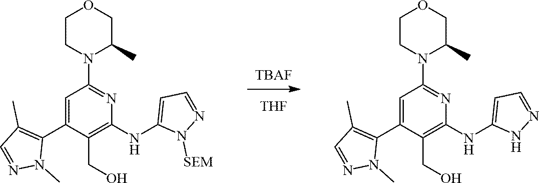 [SMILES: Cc1cnn(C)c1-c1cc(N2CCOC[C@H]2C)nc(Nc2ccnn2COCC[Si](C)(C)C)c1CO ; "TBAF" ; Cc1cnn(C)c1-c1cc(N2CCOC[C@H]2C)nc(Nc2ccn[nH]2)c1CO ; "THF"]

A mixture of [4-(1,4-dimethyl-1H-pyrazol-5-yl)-6-[(3R)-3-methylmorpholin-4-yl]-2-{[1-({[2-(trimethylsilyl)ethoxy]methyl}-λ^{∧}2-chloranyl)-1H-pyrazol-5-yl]amino}pyridin-3-yl]methanol (46 mg, 0.09 mmol) in TBAF solution (1.0 M in THF, 5 mL, 5.0 mmol) was stirred at 40° C. overnight. LC-MS showed the reaction was complete. The reaction mixture was diluted with EA (40 mL), then washed with water and brine, dried over anhydrous Na₂SO₄, filtered and concentrated. The residue was purified by Prep-HPLC (C18, 10-95% acetonitrile in H₂O with 0.1% ammonia) to give the desired product (13.2 mg, 38%). LC/MS (ESI) m/z: 384 [M+H]⁺. ¹H NMR (400 MHz, DMSO) δ 12.16 (s, 1H), 8.51 (s, 1H), 7.59 (s, 1H), 7.32 (s, 1H), 6.55 (s, 1H), 5.89 (s, 1H), 5.38 (s, 1H), 4.26 (s, 1H), 4.22 - 4.14 (m, 2H), 3.96 - 3.90 (m, 1H), 3.85 - 3.78 (m, 1H), 3.72 (d, J = 11.3 Hz, 1H), 3.64 (d, J= 2.6 Hz, 1H), 3.61 (d, J= 2.9 Hz, 1H), 3.58 (d, J = 1.6 Hz, 3H), 3.10 - 3.03 (m, 1H), 1.85 (s, 3H), 1.14 (dd, J= 6.6, 3.0 Hz, 3H).

### Example 16

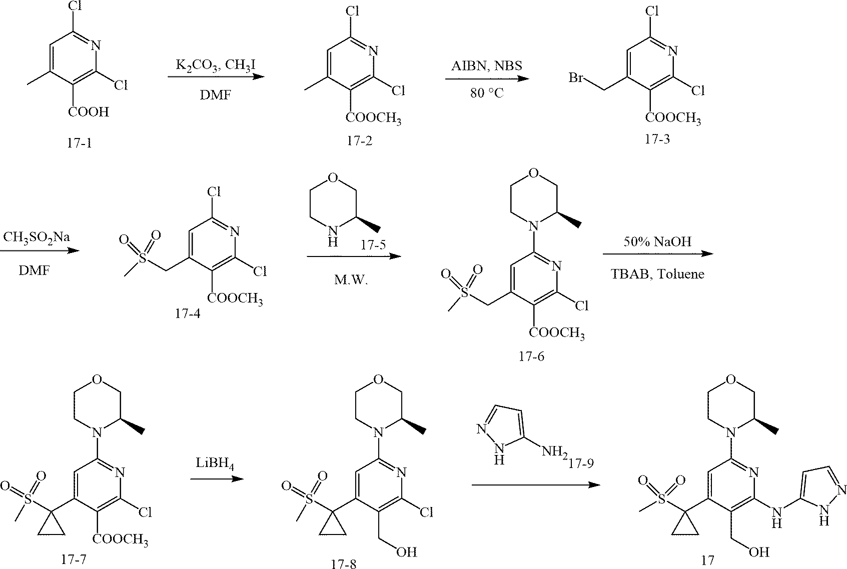

### Step 1. Methyl 2,6-dichloro-4-methylpyridine-3-carboxylate (17-2)

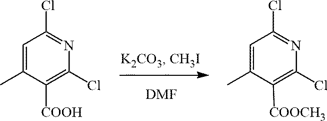

To a solution of 2,6-dichloro-4-methylpyridine-3-carboxylic acid (500 mg, 2.43 mmol) in DMF (10 mL) were added CH₃I (0.3 mL, 4.85 mmol) and K₂CO₃ (503 mg, 3.64 mmol). The mixture was stirred at room temperature overnight. LC-MS showed the reaction was complete. The reaction mixture was diluted with EA (60 mL), then washed with water and brine, dried over anhydrous Na₂SO₄, filtered and concentrated to dryness. The residue was purified by column chromatography on silica gel (PE : EA = 10:1, V/V) to give the desired product (525 mg, yield: 98%). LC/MS (ESI): m/z 220 [M+H]⁺.

### Step 2. Methyl 4-(bromomethyl)-2,6-dichloropyridine-3-carboxylate (17-3)

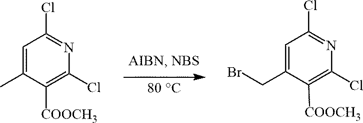

To a solution of methyl 2,6-dichloro-4-methylpyridine-3-carboxylate (1 g, 4.54 mmol) in CCl₄ (40 mL) were added NBS (0.97 g, 5.45 mmol ) and AIBN (74 mg, 0.45 mmol). The mixture was stirred at 80° C. overnight. LC-MS showed the reaction was complete. The reaction mixture was diluted with EA (60 mL), then washed with saturated Na₂S₂O₃ aqueous solution and brine, dried over anhydrous Na₂SO₄, filtered and concentrated to dryness. The residue was purified by column chromatography on silica gel (PE : EA = 10:1, V/V) to give the desired product (1.04 g, yield: 76%). LC/MS (ESI): m/z 300 [M+H]⁺.

### Step 3. Methyl 2,6-dichloro-4-(methanesulfonylmethyl)pyridine-3-carboxylate (17-4)

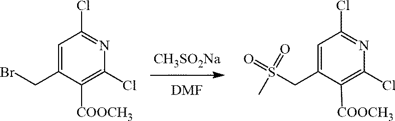

To a solution of methyl 4-(bromomethyl)-2,6-dichloropyridine-3-carboxylate (600 mg, 2.00 mmol) in DMF (20 mL) were added sodium methanesulfinate (410 mg, 4.01 mmol). The mixture was stirred at room temperature for 2 h. LC-MS showed the reaction was complete. The reaction mixture was diluted with EA (60 mL), then washed with water and brine, dried over anhydrous Na₂SO₄, filtered and concentrated to dryness. The residue was purified by column chromatography on silica gel (PE : EA= 5:1, V/V) to give the desired product (510 mg, yield: 85%). LC/MS (ESI): m/z 298 [M+H]⁺.

### Step 4. Methyl 2-chloro-4-(methanesulfonylmethyl)-6-[(3R)-3-methylmorpholin-4-yl]pyridine-3-carboxylate (17-6)

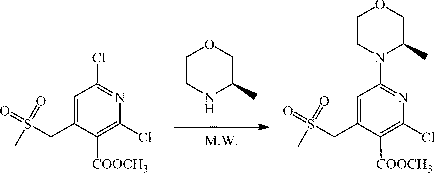

To a solution methyl 2,6-dichloro-4-(methanesulfonylmethyl) pyridine-3-carboxylate (300 mg, 1.01 mmol ) in NMP (9 mL) was added (3R)-3-methylmorpholine (204 mg, 2.01 mmol). The mixture was stirred at 120° C. for 1 h under microwave irradiation. LC-MS showed the reaction was complete. The reaction mixture was diluted with EA (60 mL), then washed with water and brine, dried over anhydrous Na₂SO₄, filtered and concentrated to dryness. The residue was purified by column chromatography on silica gel (PE : EA= 3:1, V/V) to give the desired product (150 mg, yield: 41%). LC/MS (ESI): m/z 363 [M+H]⁺.

### Step 5. Methyl 2-chloro-4-(1-methanesulfonylcyclopropyl)-6-[(3R)-3-methylmorpholin-4-yl]pyridine-3-carboxylate (17-7)

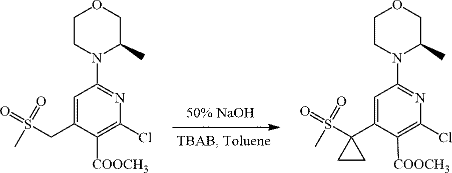

To a solution of methyl 2-chloro-4-(methanesulfonylmethyl)-6-[(3R)-3-methyl morpholin-4-yl]pyridine-3-carboxylate (150 mg, 0.41 mmol) in Toluene (20 mL) were added TBAB (27 mg, 0.08 mmol), 1,2-dibromoethane (233 mg, 1.24 mmol) and NaOH aqueous solution (10 M, 0.41 mL, 4.13 mmol). The mixture was stirred at 60° C. for 6 h. LC-MS showed the reaction was complete. The reaction mixture was diluted with EA (60 mL), then washed with water and brine, dried over anhydrous Na₂SO₄, filtered and concentrated to dryness. The residue was purified by column chromatography on silica gel (PE : EA = 3:1, V/V) to give the desired product (100 mg, yield: 62%). LC/MS (ESI): m/z 389 [M+H]⁺.

### Step 6. [2-chloro-4-(1-methanesulfonylcyclopropyl)-6-[(3R)-3-methylmorpholin-4-yl]pyridin-3-yl]methanol (17-8)

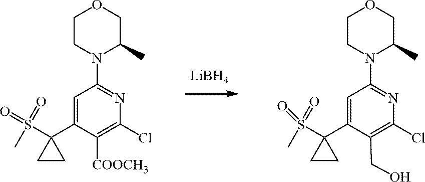 [SMILES: COC(=O)c1c(C2(S(C)(=O)=O)CC2)cc(N2CCOC[C@H]2C)nc1Cl ; "LiBH4" ; C[C@@H]1COCCN1c1cc(C2(S(C)(=O)=O)CC2)c(CO)c(Cl)n1]

To a solution of methyl 2-chloro-4-(1-methanesulfonylcyclopropyl)-6-[(3R)-3-methyl morpholin-4-yl]pyridine-3-carboxylate (200 mg, 0.51 mmol) in THF (10 mL) were added LiBH₄ (2 M in THF, 1.03 mL, 2.06 mmol). The mixture was stirred at room temperature overnight. LC-MS showed the reaction was complete. The reaction mixture was diluted with EA (60 mL), then washed with water and brine, dried over anhydrous Na₂SO₄, filtered and concentrated to dryness. The residue was purified by column chromatography on silica gel (PE : EA= 1:1, V/V) to give the desired product (150 mg, yield: 80%). LC/MS (ESI): m/z 361 [M+H]⁺.

### Step 7. (R)-(2-((1H-pyrazol-5-yl)amino)-6-(3-methylmorpholino)-4-(1-(methylsulfo- nyl)cyclopropyl)pyridin-3-yl)methanol (17)

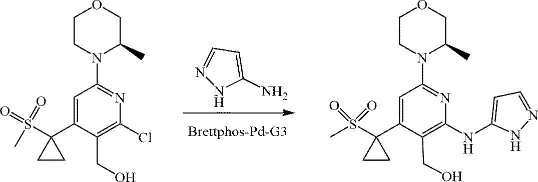

To a solution of (R)-(2-chloro-6-(3-methylmorpholino)-4-(1-(methylsulfonyl)cyclo propyl)pyridin-3-yl)methanol (50 mg, 0.14 mmol) and 1H-pyrazol-5-amine (23 mg, 0.28 mmol) in dioxane (2 mL) were added Brettphos-Pd-G3 (12.5 mmol, 0.014 mmol) and Cs₂CO₃ (135 mg, 0.41 mmol). The mixture was stirred at 110° C. overnight under N₂ atmosphere. LC-MS showed the reaction was complete. The reaction mixture was diluted with EA (40 mL), then washed with water and brine, dried over anhydrous Na₂SO₄, filtered and concentrated to dryness. The residue was purified by Prep-HPLC (C₁₈, 10-95%, MeOH in H₂O with 0.1% HCOOH) to give the desired product (14 mg, yield: 24%). LC/MS (ESI): m/z 408 [M+H]⁺. ¹H NMR (400 MHz, DMSO) δ 12.11 (s, 1H), 8.52 (d, J= 4.3 Hz, 1H), 7.56 (d, J= 1.6 Hz, 1H), 6.50 (s, 1H), 6.25 (s, 1H), 5.30 (s, 1H), 4.89 (d, J= 13.3 Hz, 1H), 4.35 (d, J= 12.8 Hz, 1H), 4.29 (s, 1H), 3.94 (d, J= 8.4 Hz, 1H), 3.77 (dd, J= 25.7, 11.3 Hz, 2H), 3.65 (s, 2H), 3.08 (d, J= 11.5 Hz, 1H), 2.96 (s, 3H), 1.84 (s, 1H), 1.49 (d, J= 28.1 Hz, 2H), 1.33 (s, 1H), 1.13 (dd, J= 18.8, 5.4 Hz, 3H).

### Example 17

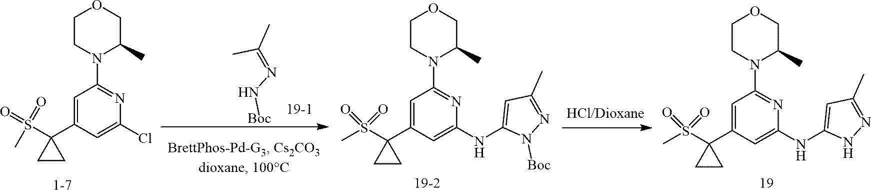

### Step 1. Tert-butyl 5-{[4-(1-methanesulfonylcyclopropyl)-6-[(3R)-3-methylmorpholin-4-yl]pyridin-2-yl] amino }-3-methyl-1H -pyrazole-l-carboxylate (19-2)

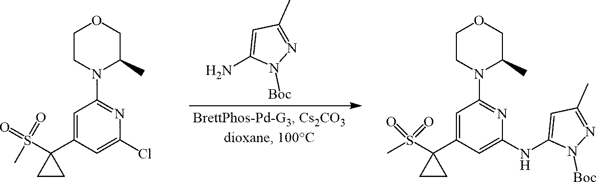

A mixture of (3R)-4-[6-chloro-4-(1-methanesulfonylcyclopropyl)pyridin-2-yl]-3-methylmorpholine (450 mg, 1.36 mmol), tert-butyl 5-amino-3-methyl-1H-pyrazole-1-carboxylate (402 mg, 2.04 mmol), BrettPhos-Pd-G3 (27 mg, 0.03 mmol) and Cs₂CO₃ (1.1 g, 3.40 mmol) in dioxane (40 mL) was stirred at 100° C. overnight under nitrogen atmosphere. LC-MS showed the reaction was complete. The mixture was diluted with EA (50 mL), then washed with water and brine, dried over anhydrous Na₂SO₄, filtered and concentrated. The residue was purified by column chromatography on silica gel (PE : EA= 1:1, V/V) to give the desired product (526 mg, yield: 79%). LC/MS ESI (m/z): 492 [M+H]⁺.

### Step 2. 4-(1-methanesulfonylcyclopropyl)-N-(3-methyl-1H-pyrazol-5-yl)-6-[(3R)-3-methylmorpholin-4-yl]pyridin-2-amine (19)

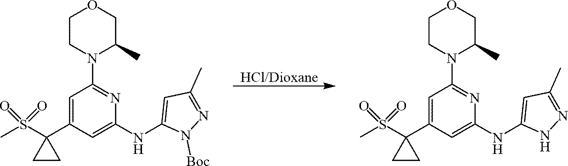

A mixture of tert-butyl 5-{[4-(1-methanesulfonylcyclopropyl)-6-[(3R)-3-methyl morpholin-4-yl]pyridin-2-yl]amino}-3-methyl-1H-pyrazole-1-carboxylate (526 mg, 1.07 mmol) in HCl solution (4 M in dioxane, 8 mL) was stirred at room temperature overnight. LC-MS showed the reaction was complete. The mixture was concentrated under reduced pressure to dryness. The residue was purified by prep-HPLC (C₁₈, 10-95%, MeOH in H₂O with 0.1% HCOOH) to give the desired product (213 mg, yield: 51%). LC/MS ESI (m/z): 392 [M+H]⁺. ¹H NMR (400 MHz, DMSO) δ 11.69 (s, 1H), 8.79 (s, 1H), 6.66 (s, 1H), 6.18 (s, 1H), 6.00 (s, 1H), 4.26 (d, J = 6.6 Hz, 1H), 3.93 (dd, J = 11.2, 3.2 Hz, 1H), 3.81 (d, J = 11.1 Hz, 1H), 3.72 (d, J = 11.2 Hz, 1H), 3.61 (dd, J = 11.3, 2.8 Hz, 1H), 3.47 (dd, J = 11.6, 8.9 Hz, 1H), 3.04 (td, J = 12.5, 3.6 Hz, 1H), 2.93 (s, 3H), 2.17 (s, 3H), 1.56 (dd, J = 5.7, 3.9 Hz, 2H), 1.24 (dd, J = 6.2, 4.7 Hz, 2H), 1.13 (d, J = 6.6 Hz, 3H).

### Example 18

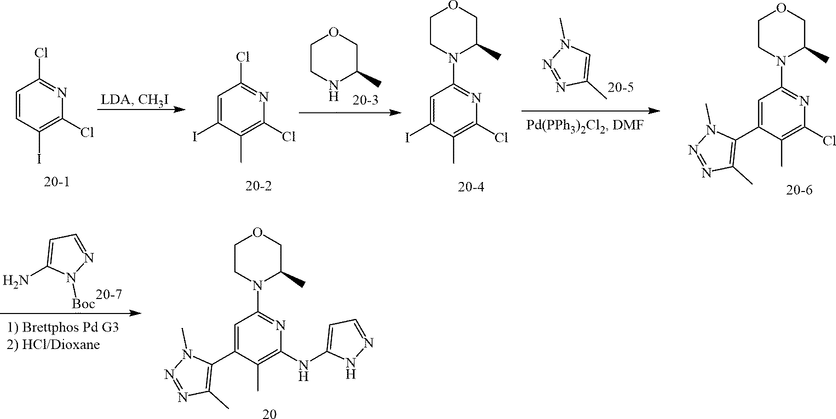

### Step 1. 2,6-dichloro-4-iodo-3-methylpyridine (20-2)

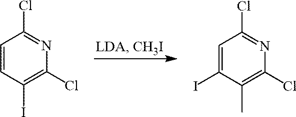

To a solution of 2,6-dichloro-3-iodopyridine (2 g, 7.30 mmol) in DMF (40 mL) at -60° C. was added LDA (2 M in THF, 5.48 mL, 10.95 mmol) drop wise. The mixture was stirred at -60° C. for 1 h, then iodomethane (0.68 mL, 10.95 mmol) was added drop wise. The resulting mixture was stirred at -60° C. for an additional 1 h. LC-MS showed the reaction was complete. The reaction was quenched with saturated NH₄Cl aqueous solution, and extracted with EA (50 mL). The organic layer was separated, then washed with brine, dried over anhydrous Na₂SO₄, filtered and concentrated. The residue was purified by flash chromatography on silica gel (PE : EA= 10:1, V/V) to afford the desired product (1.7 g, yield: 81%). LC/MS (ESI): m/z 288 [M+H]⁺.

### Step 2. (R)-4-(6-chloro-4-iodo-5-methylpyridin-2-yl)-3-methylmorpholine (20-4)

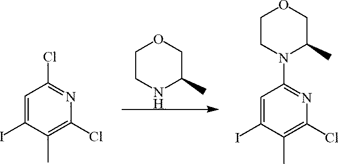

To a solution of 2,6-dichloro-4-iodo-3-methylpyridine (1.7 g, 5.90 mmol) in NMP (17.0 mL) were added (R)-3-methylmorpholine (1.79 g, 17.71 mmol) and N,N-Diisopropylethylamine (2.93 mL, 17.71 mmol). The mixture was stirred at 180° C. for 1 h under microwave irradiation. LC-MS showed the reaction was complete. The mixture was diluted with EA (60 mL), then washed with water and brine, dried over anhydrous Na₂SO₄, filtered and concentrated The residue was purified by flash chromatography on silica gel (PE : EA= 3:1, V/V) to afford the desired product (750 mg, yield: 36%). LC/MS (ESI): m/z 353 [M+H]⁺.

### Step 3. (R)-4-(6-chloro-4-(1,4-dimethyl-1H-1,2,3-triazol-5-yl)-5-methylpyridin-2-yl)-3-methylmorpholine (20-6)

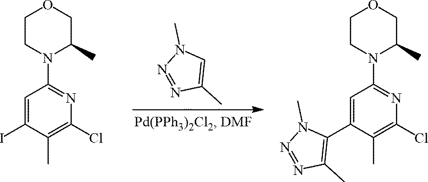

To a solution of (R)-4-(6-chloro-4-iodo-5-methylpyridin-2-yl)-3-methylmorpholine (600 mg, 1.70 mmol) in DMF (6 mL) were added 1,4-dimethyl-1H-1,2,3-triazole (182 mg, 1.87 mmol), Tetramethylammonium acetate (272 mg, 2.04 mmol) and Bis(triphenylphosphine)palladium(II) chloride (132 mg, 0.17 mmol). The mixture was stirred at 140° C. for 5 h under nitrogen atmosphere. LC-MS showed the reaction was complete. The mixture was diluted with EA (60 mL), then washed with water and brine, dried over anhydrous Na₂SO₄, filtered and concentrated. The residue was purified by flash chromatography on silica gel (PE : EA= 1:1, V/V) to afford the desired product (400 mg, yield: 73%). LC/MS (ESI): m/z 322 [M+H]⁺.

### Step 4. (R)-4-(1,4-dimethyl-1H-1,2,3-triazol-5-yl)-3-methyl-6-(3-methylmorpholino)-N-(1H-pyrazol-5-yl)pyridin-2-amine (20)

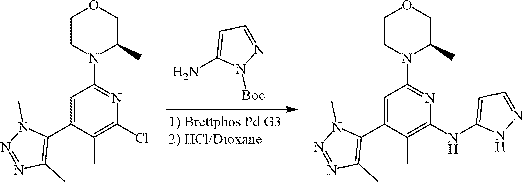

To a solution of (R)-4-(6-chloro-4-(1,4-dimethyl-1H-1,2,3-triazol-5-yl)-5-methyl pyridin-2-yl)-3-methylmorpholine (100 mg, 0.31 mmol) in Dioxane (2 mL) were added tert-butyl 5-amino-1H-pyrazole-1-carboxylate (85 mg, 0.47 mmol), Cs₂CO₃ (203 mg, 0.62 mmol) and BrettPhos-Pd-G3 (28 mg, 0.03 mmol). The mixture was stirred at 100° C. overnight under nitrogen atmosphere. LC-MS showed the reaction was complete. The mixture was diluted with EA (60 mL), then washed with water and brine, dried over anhydrous Na₂SO₄, filtered and concentrated. The residue was dissolved in DCM (5 mL), then HCl solution (4 M in dioxane, 2 mL) was added. The resulting mixture was stirred at room temperature for 12h. LC-MS showed the reaction was complete. The mixture was concentrated under reduced pressure to dryness. The residue was purified by Pre-HPLC (C18, 10-95%, MeOH in H₂O with 0.1% HCOOH) to afford the desired product (10 mg, yield: 8.7%). LC/MS (ESI): m/z 369 [M+H]⁺. ¹HNMR (400 MHz, DMSO) δ 8.13 (s, 1H), 7.56 (s, 1H), 6.46 (s, 1H), 5.97 (s, 1H), 4.24 - 4.14 (m, 1H), 3.90 (dd, J = 11.2, 3.1 Hz, 1H), 3.79 (s, 3H), 3.77 -3.71 (m, 1H), 3.69 (d, J = 11.5 Hz, 1H), 3.61 (d, J = 10.8 Hz, 1H), 3.46 (d, J = 2.6 Hz, 1H), 3.02 (t, J = 12.5 Hz, 1H), 2.10 (s, 3H), 1.82 (d, J = 0.8 Hz, 3H), 1.11 (d, J = 6.6 Hz, 3H).

### Example 19

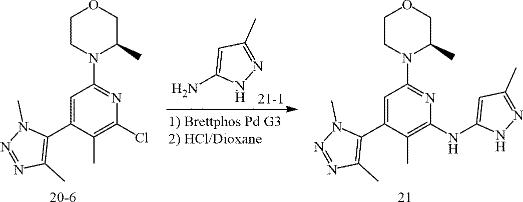

### Step 1. (R)-4-(1,4-dimethyl-1H-1,2,3-triazol-5-yl)-3-methyl-N-(3-methyl-1H-pyrazol-5-yl)-6-(3-methylmorpholino)pyridin-2-amine (21)

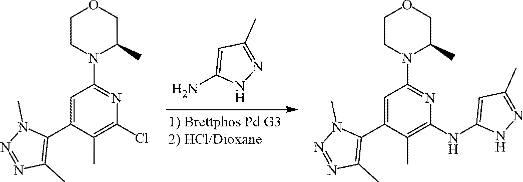

To a solution of (R)-4-(6-chloro-4-(1,4-dimethyl-1H-1,2,3-triazol-5-yl)-5-methyl pyridin-2-yl)-3-methylmorpholine (100 mg, 0.31 mmol) in dioxane (2 mL) were added 3-methyl-1H-pyrazol-5-amine (45 mg, 0.47 mmol), Cs₂CO₃ (203 mg, 0.62 mmol) and BrettPhos-Pd-G3 (28 mg, 0.03 mmol). The mixture was stirred at 100° C. overnight under nitrogen atmosphere. LC-MS showed the reaction was complete. The mixture was diluted with EA (60 mL), then washed with water and brine, dried over anhydrous Na₂SO₄, filtered and concentrated. The residue was dissolved in DCM (5 mL), then HCl solution (4 M in dioxane, 2 mL) was added. The resulting mixture was stirred at room temperature for 12h. LC-MS showed the reaction was complete. The mixture was concentrated under reduced pressure to dryness. The residue was purified by Pre-HPLC (C18, 10-95%, MeOH in H₂O with 0.1% HCOOH) to afford the desired product (15 mg, yield: 12.6%). LC/MS (ESI) m/z: 383 [M+H]⁺. ¹HNMR (400 MHz, DMSO) δ 8.00 (s, 1H), 6.22 (s, 1H), 5.96 (s, 1H), 4.20 (s, 1H), 3.91 (dd, J = 11.2, 3.1 Hz, 1H), 3.78 (s, 3H), 3.77 - 3.72 (m, 1H), 3.70 (d, J = 11.8 Hz, 1H), 3.61 (d, J = 10.8 Hz, 1H), 3.50 - 3.43 (m, 1H), 3.06 - 2.98 (m, 1H), 2.20 (s, 3H), 2.09 (s, 3H), 1.80 (d, J = 0.7 Hz, 3H), 1.12 (d, J = 6.6 Hz, 3H).

### Example 20

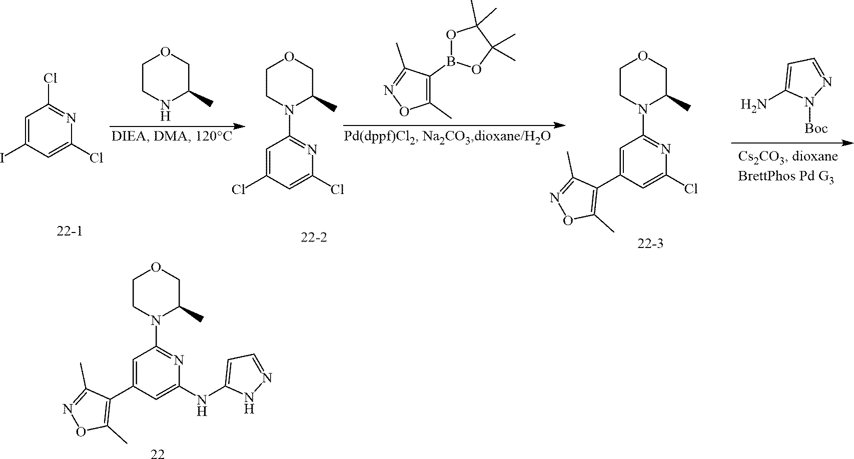

### Step 1: (R)-4-(4,6-dichloropyridin-2-yl)-3-methylmorpholine (22-2)

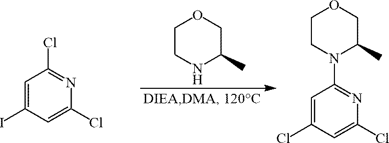

To a solution of 2,6-dichloro-4-iodopyridine (800 mg, 2.92 mmol) and (R)-3-methylmorpholine (325 mg, 3.21 mmol) in DMA (8 mL) were added DIEA (755 mg, 5.84 mmol). The mixture was charged with N₂ twice, then stirred at 120° C. for 12 hrs. The reaction mixture was diluted with water (30 mL) and extracted with EA (30 mL × 2). The combined organic layer was washed with brine (50 mL), dried over anhydrous Na₂SO₄, filtered and concentrated in vacuo. The resulting mixture was purified by flash chromatography eluting with PE/EtOAc (20:1, 8:1) to afford the desired product (500 mg, yield: 68.9%).

### Step 2: (R)-4-(6-chloro-4-(3,5-dimethylisoxazol-4-yl)pyridin-2-yl)-3-methylmorpholine (22-3)

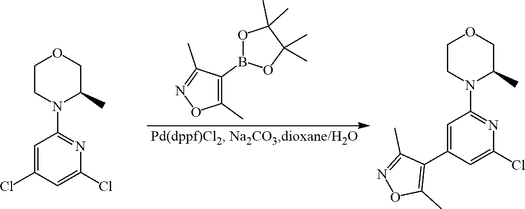

To a solution of (R)-4-(6-chloro-4-iodopyridin-2-yl)-3-methylmorpholine (300 mg, 1.21 mmol) and 3,5-dimethyl-4-(4,4,5,5-tetramethyl-1,3,2-dioxaborolan-2-yl)isoxazole (270.8 mg, 1.21 mmol) in dioxane (10 mL) were added Na₂CO₃ (320 mg, 3.03 mmol) and Pd(dppf)Cl₂ (88 mg, 0.12 mmol). The mixture was charged with N₂ twice, then stirred at 90° C. for 12 hrs. The reaction mixture was diluted with water (30 mL) and extracted with EA (30 mL × 2). The combined organic layer was washed with brine (50 mL), dried over anhydrous Na₂SO₄, filtered and concentrated in vacuo. The resulting mixture was purified by flash chromatography eluting with PE/EtOAc (3:1, 1:1) to afford the desired product (320 mg, yield: 86.02%).

### Step 3: (R)-4-(3,5-dimethylisoxazol-4-yl)-6-(3-methylmorpholino)-N-(1H-pyrazol-5-yl)pyridin-2-amine (22)

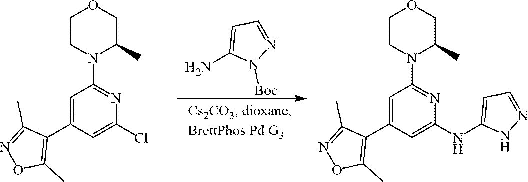

To a (R)-4-(6-chloro-4-(3,5-dimethylisoxazol-4-yl)pyridin-2-yl)-3-methylmorpholine (150 mg, 0.49 mmol) and tert-butyl 5-amino-1H-pyrazole-1-carboxylate (108 mg, 0.59 mmol) in dioxane (8 mL) were added Cs₂CO₃ (400 mg, 1.23 mmol) and BrettPhos Pd G3 (45 mg, 0.049 mmol). The mixture was charged with N₂ twice, then stirred at 90° C. overnight. The reaction was diluted with water and extracted with EtOAc (30 mL×2). The combined organic layer was washed with brine (50 mL), dried over anhydrous Na₂SO₄, filtered and concentrated in vacuo. The residue was purified by Pre-TLC (DCM/MeOH=10/1) to afford the desired product (20 mg, yield 11.49%). LC/MS (ESI) m/z: 355.0 [M+H]⁺.

¹H NMR (400 MHz, DMSO-d6) δ ppm 12.03 (br s, 1 H) 8.98 (br s, 1 H) 7.55 (br s, 1 H) 6.33 - 6.47 (m, 2 H) 5.99 (s, 1 H) 4.30 (br d, J=5.16 Hz, 1 H) 3.94 (br dd, J=11.12, 3.20 Hz, 1 H) 3.84 (br d, J=11.68 Hz, 1 H) 3.71 - 3.75 (m, 1 H) 3.61 - 3.66 (m, 1 H) 3.45 - 3.52 (m, 1 H) 3.03 - 3.10 (m, 1 H) 2.44 (s, 3 H) 2.25 (s, 3 H) 1.15 (d, J=6.64 Hz, 3 H).

### Example 21

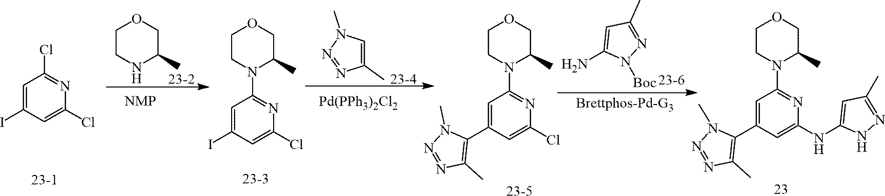

### Step 1. (3R)-4-(6-chloro-4-iodopyridin-2-yl)-3-methylmorpholine (23-3)

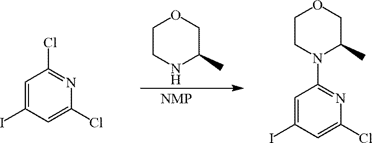

To a solution of 2,6-dichloro-4-iodopyridine (500 mg, 1.83 mmol) in NMP (10 mL) were added (3R)-3-methylmorpholine (554.1 mg, 5.48 mmol). The mixture was stirred at 150° C. for 1 h under microwave irradiation. The reaction mixture was diluted with EA (60 mL), then washed with water and brine, dried over anhydrous Na₂SO₄, filtered and concentrated to dryness. The residue was purified by column chromatography on silica gel (PE : EA= 10:1, V/V) to give the desired product (250 mg, yield: 40%). LC/MS (ESI): m/z 339 [M+H]⁺.

### Step 2. (3R)-4-[6-chloro-4-(dimethyl-1H-1,2,3-triazol-5-yl)pyridin-2-yl]-3-methyl morpholine (23-5)

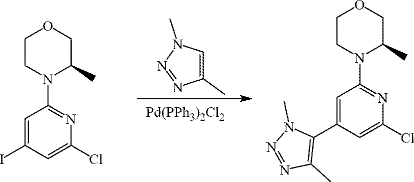

To a solution of (3R)-4-(6-chloro-4-iodopyridin-2-yl)-3-methylmorpholine (300 mg, 0.88 mmol) and 1,4-dimethyl-1H-1,2,3-triazole (103.3 mg, 1.06 mmol) in DMF (15 mL) were added Pd(PPh₃)₂Cl₂ (62.2 mg, 0.09 mmol) and tetramethylammonium acetate (141.6 mg, 1.06 mmol). The mixture was stirred at 100° C. overnight under nitrogen atmosphere. LC-MS showed the reaction was complete. The reaction mixture was diluted with DCM (60 mL), then washed with water and brine, dried over anhydrous Na₂SO₄, filtered and concentrated to dryness. The residue was purified by column chromatography on silica gel (PE : EA= 3:1, V/V) to give the desired product (210 mg, yield: 77%). LC/MS (ESI): m/z 309 [M+H]⁺.

### Step 3. 4-(dimethyl-1H-1,2,3-triazol-5-yl)-N-(3-methyl-1H-pyrazol-5-yl)-6-[(3R)-3 -methylmorpholin-4-yl]pyridin-2-amine (23)

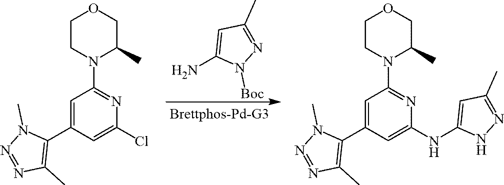 [SMILES: Cc1nnn(C)c1-c1cc(Cl)nc(N2CCOC[C@H]2C)c1 ; "Brettphos-Pd-G3" ; Cc1cc(N)n(C(=O)OC(C)(C)C)n1 ; Cc1cc(Nc2cc(-c3c(C)nnn3C)cc(N3CCOC[C@H]3C)n2)[nH]n1]

To a solution of (3R)-4-[6-chloro-4-(dimethyl-1H-1,2,3-triazol-5-yl)pyridin-2-yl]-3-methylmorpholine (90 mg, 0.29 mmol) in dioxane (2 mL) were added tert-butyl 5-amino-3-methyl-1H-pyrazole-1-carboxylate (86.51 mg, 0.439 mmol), BrettPhos-Pd -G3 (26.5 mg, 0.03 mmol) and Cs₂CO₃ (190.5 mg, 0.59 mmol). The mixture was stirred at 100° C. overnight under nitrogen atmosphere. LC-MS showed the reaction was complete. The reaction mixture was diluted with EA (40 mL), then washed with water and brine, dried over anhydrous Na₂SO₄, filtered and concentrated to dryness. The residue was purified by Prep-HPLC (C18, 10-95% MeCN in H₂O with 0.1% ammonia) to give the desired product (35 mg, yield: 32 %). LC/MS (ESI): m/z 369 [M+H]⁺. ¹H NMR (400 MHz, DMSO) δ 11.71 (s, 1H), 8.92 (s, 1H), 6.55 (s, 1H), 6.07 (d, J= 6.1 Hz, 2H), 4.31 (d, J=5.9 Hz, 1H), 3.96 (s, 3H), 3.90 (dd, J= 14.1, 8.1 Hz, 2H), 3.72 (d, J= 11.2 Hz, 1H), 3.63 (dd, J= 11.2, 2.9 Hz, 1H), 3.48 (td, J= 11.8, 2.8 Hz, 1H), 3.07 (td, J= 12.5, 3.5 Hz, 1H), 2.25 (s, 3H), 2.18 (s, 3H), 1.16 (d, J= 6.6 Hz, 3H).

### Example 22

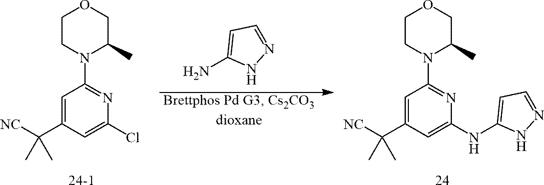

### Step 1. (R)-2-(2-((1H-pyrazol-5-yl)amino)-6-(3-methylmorpholino)pyridin-4-yl)-2-methylpropanenitrile (24)

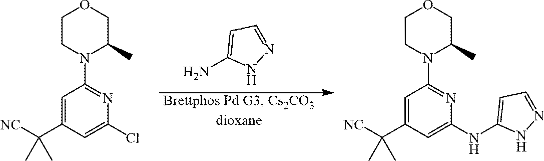

To a solution of 2-{2-chloro-6-[(3R)-3-methylmorpholin-4-yl]pyridin-4-yl}-2-methylpropanenitrile (60 mg, 0.21 mmol) and 1H-pyrazol-5-amine (35 mg, 0.42 mmol) in Dioxane (3 mL) were added BrettPhos-Pd-G3 (19 mg, 0.21 mmol) and Cs₂CO₃ (210 mg, 0.64 mmol). The mixture was stirred at 110° C. for 10 h under N₂ atmosphere. LC-MS showed the reaction was complete. The reaction mixture was diluted with EA (40 mL), then washed with water and brine, dried over anhydrous Na₂SO₄, filtered and concentrated. The residue was purified by Prep-HPLC (C₁₈, 10-95%, MeOH in H₂O with 0.1% HCOOH) to give the desired product (60 mg, yield: 85%). LC/MS (ESI): m/z 327 [M+H]⁺. ¹H NMR (400 MHz, DMSO) δ 12.10 (s, 1H), 9.02 (s, 1H),7.53 (d, J = 2.2 Hz, 1H), 6.59 (s, 1H), 6.30 (d, J = 1.9 Hz, 1H), 6.08 (d, J = 1.0 Hz, 1H), 4.32 (d, J = 6.5 Hz, 1H), 3.93 (dd, J = 11.2, 3.3 Hz, 1H), 3.79 (d, J = 12.8 Hz, 1H), 3.73 (d, J = 11.2 Hz, 1H), 3.62 (dd, J = 11.3, 2.9 Hz, 1H), 3.47 (td, J = 11.8, 3.0 Hz, 1H), 3.06 (td, J = 12.6, 3.7 Hz, 1H), 1.63 (s, 6H), 1.13 (d, J = 6.6 Hz, 3H).

### Example 23

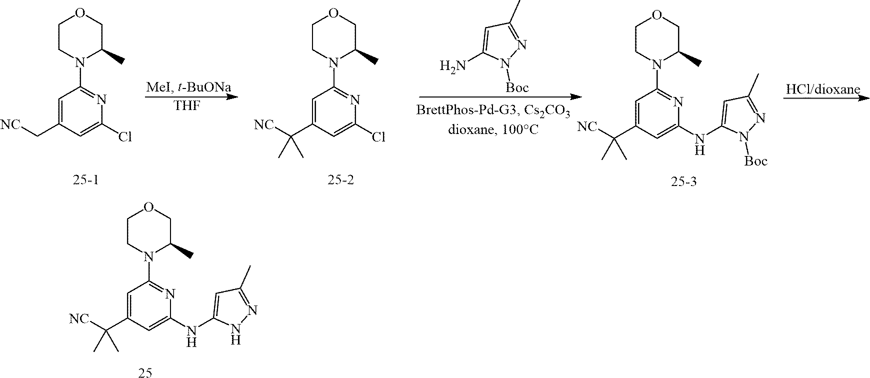

### Step 1. 2-{2-chloro-6-[(3R)-3-methylmorpholin-4-yl]pyridin-4-yl}-2-methylpropane nitrile (25-2)

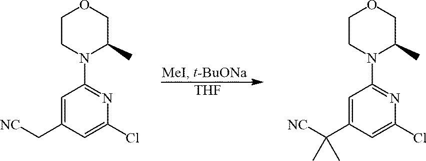

To a solution of 2-{2-chloro-6-[(3R)-3-methylmorpholin-4-yl]pyridin-4-y1}acetonitrile (173 mg, 0.69 mmol) in THF (6 mL) at 0° C. was added Sodium tert-butoxide (198 mg, 2.06 mmol) and iodomethane (0.13 mL, 2.06 mmol). The mixture was stirred at ambient temperature overnight. LC-MS showed the reaction was complete. The mixture was diluted with EA (50 mL), then washed with water and brine, dried over anhydrous Na₂SO₄, filtered and concentrated. The residue was purified by column chromatography on silica gel (PE : EA = 3:1, V/V) to give the desired product (154 mg, yield: 80%). LC/MS ESI (m/z): 280 [M+H]⁺.

### Step 2. Tert-butyl 5-{[4-(1-cyano-1-methylethyl)-6-[(3R)-3-methylmorpholin-4-yl]pyridin-2-yl]amino}-3-methyl-1H-pyrazole-1-carboxylate (25-3)

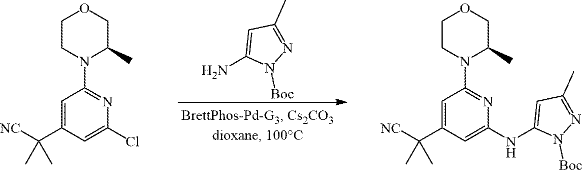

To a solution of 2-{2-chloro-6-[(3R)-3-methylmorpholin-4-yl]pyridin-4-yl}-2-methyl propanenitrile (154 mg, 0.55 mmol) and tert-butyl 5-amino-3-methyl-1H-pyrazole-1-carboxylate (163 mg, 0.83 mmol) in dioxane (10 mL) were added BrettPhos-Pd-G3 (50 mg, 0.06 mmol) and Cs₂CO₃ (538 mg, 1.65 mmol). The mixture was stirred at 100° C. overnight under nitrogen atmosphere. LC-MS showed the reaction was complete. The mixture was diluted with EA (50 mL), then washed with water and brine, dried over anhydrous Na₂SO₄, filtered and concentrated. The residue was purified by column chromatography on silica gel (PE : EA= 1:1, V/V) to give the desired product (156 mg, yield: 64%). LC/MS ESI (m/z): 441 [M+H]⁺.

### Step 3. 2-methyl-2-{2-[(3-methyl-1H-pyrazol-5-yl)amino]-6-[(3R)-3-methyl morpholin-4-yl]pyridin-4-yl}propanenitrile (25)

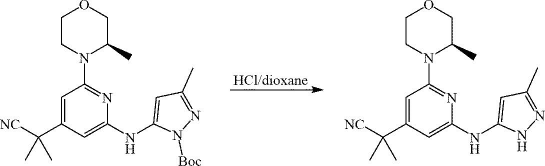

A mixture of tert-butyl 5-{[4-(1-cyano-1-methylethyl)-6-[(3R)-3-methylmorpholin-4-yl]pyridin-2-yl]amino}-3 -methyl-1H-pyrazole-1-carboxylate (156 mg, 0.35 mmol) in HCl solution (4 M in dioxane, 4 mL) was stirred at room temperature overnight. LC-MS showed the reaction was complete. The mixture was concentrated under reduced pressure to dryness. The residue was purified by prep-HPLC (C₁₈, 10-95%, MeOH in H₂O with 0.1% HCOOH) to give the desired product (88.5 mg, yield: 73%). LC/MS ESI (m/z): 341 [M+H]⁺. ¹H NMR (400 MHz, DMSO) δ 9.43 (s, 1H), 6.60 (s, 1H), 6.18 (s, 1H), 6.04 (s, 1H), 4.30 (dd, J = 6.6, 1.9 Hz, 1H), 3.95 (dd, J = 11.3, 3.4 Hz, 1H), 3.80 (dd, J = 13.1, 2.0 Hz, 1H), 3.74 (d, J = 11.3 Hz, 1H), 3.64 (dd, J = 11.3, 2.8 Hz, 1H), 3.49 (td, J = 11.8, 3.0 Hz, 1H), 3.11 (td, J = 12.6, 3.8 Hz, 1H), 2.21 (s, 3H), 1.64 (s, 6H), 1.15 (d, J = 6.6 Hz, 3H).

### Example 24

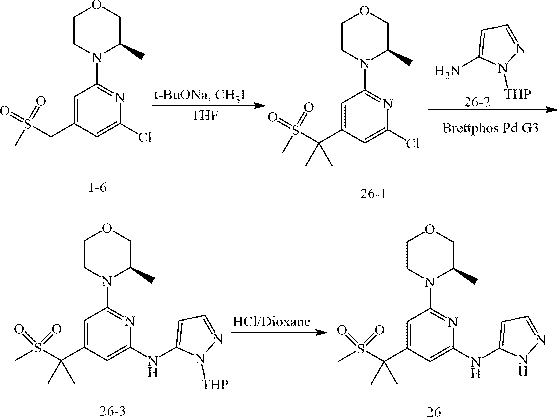

### Step 1. (R)-4-(6-chloro-4-(2-(methylsulfonyl)propan-2-yl)pyridin-2-yl)-3-methyl morpholine (26-1)

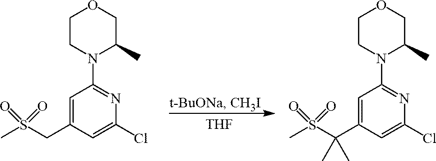

To a solution of (3R)-4-[6-chloro-4-(methanesulfonylmethyl)pyridin-2-yl]-3-methyl morpholine (5.8 g, 19.03 mmol) in THF (100 mL) were added CH₃I (4.7 mL, 76.11 mmol) and t-BuONa (7.31 g, 76.11 mmol). The reaction was stirred at room temperature overnight. The reaction was diluted with EA (100 mL), then washed with water and brine, dried over anhydrous Na₂SO₄, filtered and concentrated. The residue was purified by column chromatography on silica gel (PE:EA=1:1, V/V) to give the desired product (5.3 g, yield: 83.7%). LC/MS (ESI): m/z 333 [M+H]⁺.

### Step 2. 6-((R)-3-methylmorpholino)-4-(2-(methylsulfonyl)propan-2-yl)-N-(1-(tetra hydro-2H-pyran-2-yl)-1H-pyrazol-5-yl)pyridin-2-amine (26-3)

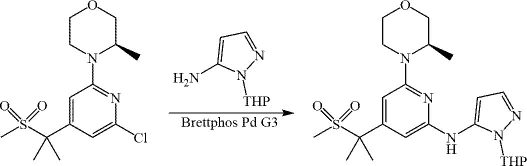

To a solution of (3R)-4-[6-chloro-4-(2-methanesulfonylpropan-2-yl)pyridin-2-yl]-3-methylmorpholine (4.0 g, 12.02 mmol ) in Dioxane (80 mL) were added 1-(oxan-2-yl)-1H-pyrazol-5-amine (3.0 g, 18.03 mmol), Brettphos Pd G3 (1.09 g, 1.20 mmol), and Cs₂CO₃ (11.8 g, 36.05 mmol). The mixture was stirred at 100° C. overnight under nitrogen atmosphere. The reaction was diluted with DCM (100 mL), then washed with water and brine, dried over anhydrous Na₂SO₄, filtered and concentrated. The residue was purified by column chromatography on silica gel (DCM:MeOH=30:1, V/V) to give the desired product (4.45 g, yield: 80%). LC/MS (ESI): m/z 464 [M+H]⁺.

### Step 3. (R)-6-(3-methylmorpholino)-4-(2-(methylsulfonyl)propan-2-yl)-N-(1H-pyrazol-5-yl)pyridin-2-amine (26)

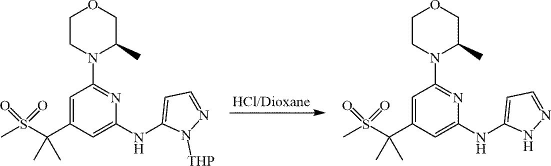

To a solution of 4-(2-methanesulfonylpropan-2-yl)-6-[(3R)-3-methylmorpholin-4-yl]-N-[1-(oxan-2-yl)-1H-pyrazol-5-yl]pyridin-2-amine (4.45 g, 9.60 mmol) in DCM (50 mL) were added HCl/dioxane (50 mL). The reaction was stirred at room temperature overnight. The reaction mixture was concentrated under vacuo. The residue was diluted with DCM (50 mL), then washed with saturated NaHCO₃ aqueous solution and brine, dried over anhydrous Na₂SO₄, filtered and concentrated. The residue was purified by Prep-HPLC (C₁₈, 10-95%, MeOH in H₂O with 0.1% HCOOH) to give the desired product (2.08 g, 57 %). LC/MS (ESI): m/z 380 [M+H]⁺. ¹H NMR (400 MHz, DMSO) δ 12.02 (s, 1H), 8.95 (s, 1H), 7.53 (s, 1H), 6.65 (s, 1H), 6.27 (d, J= 60.7 Hz, 1H), 6.19 (s, 1H), 4.28 (d, J = 6.2 Hz, 1H), 3.94 (dd, J= 11.1, 3.2 Hz, 1H), 3.76 (dd, J= 22.8, 11.2 Hz, 2H), 3.63 (dd, J= 11.2, 2.8 Hz, 1H), 3.48 (td, J= 11.7, 2.9 Hz, 1H), 3.05 (td, J= 12.6, 3.7 Hz, 1H), 2.76 (s, 3H), 1.66 (s, 6H), 1.12 (d, J= 6.6 Hz, 3H).

### Example 25

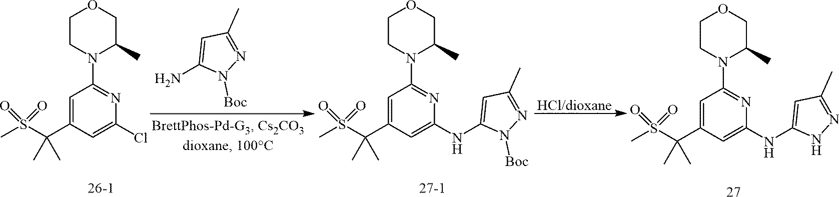

### Step 1. Tert-butyl 5-{[4-(2-methanesulfonylpropan-2-yl)-6-[(3R)-3-methyl morpholin-4-yl]pyridin-2-yl]amino}-3-methyl-1H-pyrazole-1-carboxylate (27-1)

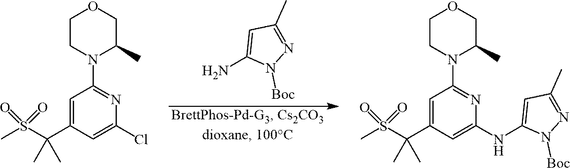

A mixture of (3R)-4-[6-chloro-4-(2-methanesulfonylpropan-2-yl)pyridin-2-yl]-3-methylmorpholine (580 mg, 1.74 mmol), tert-butyl 5-amino-3-methyl-1H-pyrazole-1-carboxylate (516 mg, 2.61 mmol), BrettPhos-Pd-G3 (157.9 mg, 0.17 mmol) and Cs₂CO₃ (1.42 g, 4.36 mmol) in dioxane (20 mL) was stirred at 100° C. overnight under nitrogen atmosphere. LC-MS showed the reaction was complete. The mixture was diluted with EA (50 mL), then washed with water and brine, dried over anhydrous Na₂SO₄, filtered and concentrated. The residue was purified by column chromatography on silica gel (PE : EA = 1:1, V/V) to give the desired product (758 mg, yield: 88%). LC/MS ESI (m/z): 494 [M+H]⁺.

### Step 2. 4-(2-methanesulfonylpropan-2-yl)-N-(3-methyl-1H-pyrazol-5-yl)-6-[(3R)-3-methylmorpholin-4-yl]pyridin-2-amine (27)

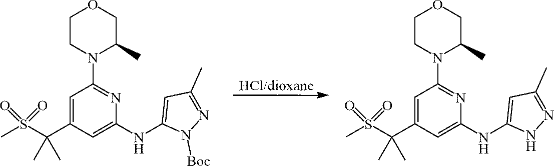

A mixture of tert-butyl 5-{[4-(2-methanesulfonylpropan-2-yl)-6-[(3R)-3-methyl morpholin-4-yl]pyridin-2-yl]amino}-3-methyl-1H-pyrazole-1-carboxylate (758 mg, 1.54 mmol) in HCl solution (4 M in dioxane, 8 mL) was stirred at ambient temperature overnight. LC-MS showed the reaction was complete. The mixture was concentrated under reduced pressure to dryness. The residue was purified by prep-HPLC (C₁₈, 10-95%, MeOH in H₂O with 0.1% HCOOH) to give the desired product (215 mg, yield: 35%). LC/MS ESI (m/z): 394 [M+H]⁺. ¹H NMR (400 MHz, DMSO) δ 11.67 (s, 1H), 8.79 (s, 1H), 6.70 (s, 1H), 6.19 (s, 1H), 6.03 (s, 1H), 4.27 (d, J = 6.6 Hz, 1H), 3.94 (dd, J = 11.2, 3.2 Hz, 1H), 3.79 (d, J = 13.1 Hz, 1H), 3.73 (d, J = 11.2 Hz, 1H), 3.63 (dd, J = 11.2, 2.8 Hz, 1H), 3.48 (td, J = 11.8, 2.9 Hz, 1H), 3.04 (td, J = 12.6, 3.7 Hz, 1H), 2.75 (s, 3H), 2.17 (s, 3H), 1.66 (s, 6H), 1.12 (d, J = 6.6 Hz, 3H).

### Example 26

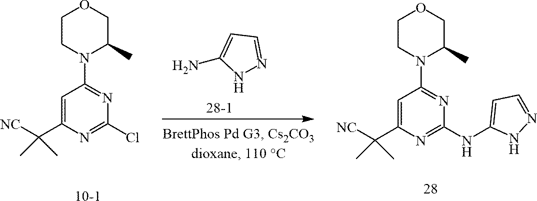

### Step 1. (R)-2-(2-((1H-pyrazol-5-yl)amino)-6-(3-methylmorpholino)pyrimidin-4-yl)-2-methylpropanenitrile (28)

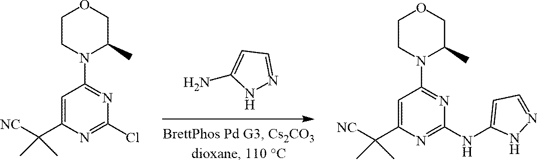

A mixture of 2-{2-chloro-6-[(3R)-3-methylmorpholin-4-yl]pyrimidin-4-yl}-2-methylpropanenitrile (100 mg, 0.35 mmol), 1H-pyrazol-5-amine (59 mg, 0.71 mmol), BrettPhos Pd G3 (32 mg, 0.03 mmol) and Cs₂CO₃ (349 mg, 1.07 mmol) in dioxane (4 mL) was stirred at 110° C. for 16 h under N₂ atmosphere. LC-MS showed the reaction was complete. The reaction mixture was diluted with EA (40 mL), then washed with water and brine, dried over anhydrous Na₂SO₄, filtered and concentrated under vacuo. The residue was purified by Prep-HPLC (C₁₈, 10-95%, MeOH in H₂O with 0.1% HCOOH) to give the desired product (36 mg, yield: 31%). LC/MS (ESI): m/z 328 [M+H]⁺. ¹H NMR (400 MHz, DMSO) δ 12.08 (s, 1H), 9.20 (s, 1H), 8.13 (s, 1H), 7.51 (s, 1H), 6.52 (s, 1H), 6.25 (s, 1H), 4.41 (s, 1H), 4.01 (d, J = 12.8 Hz, 1H), 3.93 (dd, J = 11.3, 3.4 Hz, 1H), 3.72 (d, J = 11.4 Hz, 1H), 3.58 (dd, J = 11.4, 3.0 Hz, 1H), 3.43 (td, J = 11.8, 2.9 Hz, 1H), 3.14 (td, J = 13.0, 3.8 Hz, 1H), 1.65 (s, 6H), 1.19 (d, J = 6.7 Hz, 3H).

### Example 27

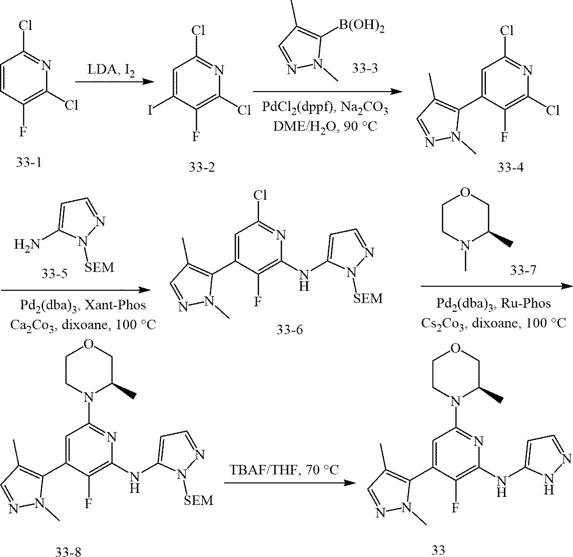

### Step 1. 2,6-dichloro-3-fluoro-4-iodopyridine (33-2)

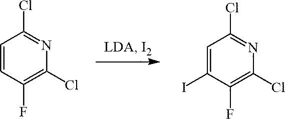

To a solution of 2,6-dichloro-3-fluoropyridine (2.0 g, 12.05 mmol) in anhydrous THF (30 mL) at -78° C. was added LDA (2.0 M in THF, 6.6 mL, 13.2 mmol) drop wise under N₂ atmosphere. The mixture was stirred at -78° C. for 1 h, then a solution of I₂ (4.0 g, 15.74 mmol) in anhydrous THF (10 mL) was added drop wise. The resulting mixture was stirred at -78° C. for an additional 1 h. LC-MS showed the reaction was complete. The reaction mixture was quenched with saturated NH₄Cl aqueous solution and diluted with EA (30 mL×3). The combined organic layer was washed with saturated Na₂S₂O₃ aqueous solution and brine, dried over anhydrous Na₂SO₄, filtered and concentrated under vacuo. The residue was purified by column chromatography on silica gel (PE) to give the desired product (2.79 g, yield: 79%). ¹H NMR (400 MHz, DMSO) δ 8.16 (d, J = 3.5 Hz, 1H).

### Step 2. 2,6-dichloro-4-(1,4-dimethyl-1H-pyrazol-5-yl)-3-fluoropyridine (33-4)

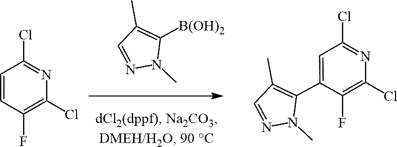

A mixture of 2,6-dichloro-3-fluoro-4-iodopyridine (1.0 g, 3.42 mmol), (1,4-dimethyl-1H-pyrazol-5-yl)boronic acid (0.76 g, 3.43 mmol), PdCl₂(dppf) (251 mg, 0.34 mmol) and Na₂CO₃ (2.0 M in H₂O, 3.4 mL) in DME (35 mL) was stirred at 90° C. for 15 h under N₂ atmosphere. LC-MS showed the reaction was complete. The reaction mixture was diluted with EA (40 mL), then washed with water and brine, dried over anhydrous Na₂SO₄, filtered and concentrated. The residue was purified by column chromatography on silica gel (PE : EA = 10:1, V/V) to give the desired product (744 mg, yield: 83%). LC/MS (ESI): m/z 260 [M+H]⁺.

### Step 3. 6-chloro-4-(1,4-dimethyl-1H-pyrazol-5-yl)-3-fluoro-N-(1-((2-(trimethylsilyl) ethoxy)methyl)-1H-pyrazol-5-yl)pyridin-2-amine (33-6)

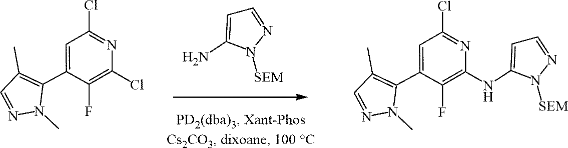

A mixture of 2,6-dichloro-4-(1,4-dimethyl-1H-pyrazol-5-yl)-3-fluoropyridine (400 mg, 1.53 mmol), 1-{[2-(trimethylsilyl)ethoxy]methyl }-1H-pyrazol-5-amine (329 mg, 1.54 mmol), Pd₂(dba)₃ (141 mg, 0.15 mmol), XantPhos (89 mg, 0.15 mmol) and Cs₂CO₃ (1.0 g, 3.06 mmol) in dioxane (25 mL) was stirred at 100° C. for 6 h under N₂ atmosphere. LC-MS showed the reaction was complete. The reaction mixture was diluted with EA (40 mL), then washed with water and brine, dried over anhydrous Na₂SO₄, filtered and concentrated. The residue was purified by column chromatography on silica gel (PE : EA= 2:1, V/V) to give the desired product (419 mg, yield: 62%). LC/MS (ESI): m/z 437 [M+H]⁺.

### Step 4. (R)-4-(1,4-dimethyl-1H-pyrazol-5-yl)-3-fluoro-6-(3-methylmorpholino)-N-(1-((2-(trimethylsilyl)ethoxy)methyl)-1H-pyrazol-5-yl)pyridin-2-amine (33-8)

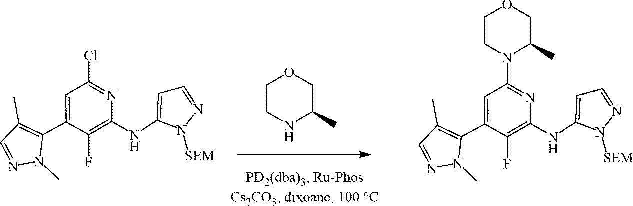

A mixture of 6-chloro-4-(1,4-dimethyl-1H-pyrazol-5-yl)-3-fluoro-N-(1-{ [2-(trimethyl silyl)ethoxy]methyl}-1H-pyrazol-5-yl)pyridin-2-amine (400 mg, 0.91 mmol), (3R)-3-methylmorpholine (278 mg, 2.74 mmol), Pd₂(dba)₃ (168 mg, 0.18 mmol), RuPhos (171 mg, 0.36 mmol) and Cs₂CO₃ (1.19 g, 3.65 mmol) in dioxane (40 mL) was stirred at 100° C. for 6 h under N₂ atmosphere. LC-MS showed the reaction was complete. The reaction mixture was diluted with EA (40 mL), then washed with water and brine, dried over anhydrous Na₂SO₄, filtered and concentrated. The residue was purified by column chromatography on silica gel (PE : EA= 2:1, V/V) to give the desired product (437 mg, yield: 95%). LC/MS (ESI): m/z 502 [M+H]⁺.

### Step 5. (R)-4-(1,4-dimethyl-1H-pyrazol-5-yl)-3-fluoro-6-(3-methylmorpholino)-N-(1H-pyrazol-5-yl)pyridin-2-amine (33)

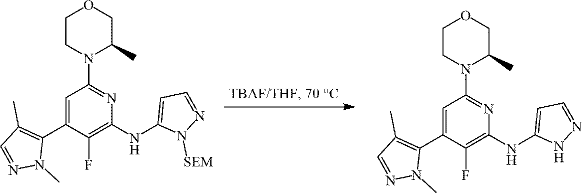

A mixture of 4-(1,4-dimethyl-1H-pyrazol-5-yl)-3-fluoro-6-[(3R)-3-methylmorpholin-4-yl]-N-(1-{[2-(trimethylsilyl)ethoxy]methyl}-1H-pyrazol-5-yl)pyridin-2-amine (417 mg, 0.83 mmol) in TBAF solution (1.0 M in THF, 8 mL, 8 mmol) was stirred at 70° C. for 5 h. LC-MS showed the reaction was complete. The reaction mixture was diluted with EA (40 mL), then washed with water (20 mL×2) and brine, dried over anhydrous Na₂SO₄, filtered and concentrated. The residue was purified by column chromatography on silica gel (PE : EA = 1:1, V/V) to obtain a brown solid (252 mg), which was further purified by Prep-HPLC (C₁₈, 10-95%, MeOH in H₂O with 0.1% HCOOH) to give the desired product (66.9 mg, yield: 21%). LC/MS (ESI): m/z 372 [M+H]⁺. ¹H NMR (400 MHz, DMSO) δ 12.18 (s, 1H), 8.89 (s, 1H), 7.59 (s, 1H), 7.37 (s, 1H), 6.51 (s, 1H), 5.94 (s, 1H), 4.20 (d, J = 4.8 Hz, 1H), 3.97 - 3.89 (m, 1H), 3.77 = 3.69 (m, 5H), 3.63 (dd, J = 11.2, 2.7 Hz, 1H), 3.51 (s, 1H), 3.05 (td, J = 12.6, 3.7 Hz, 1H), 1.96 (s, 3H), 1.13 (d, J = 6.6 Hz, 3H).

### Example 28

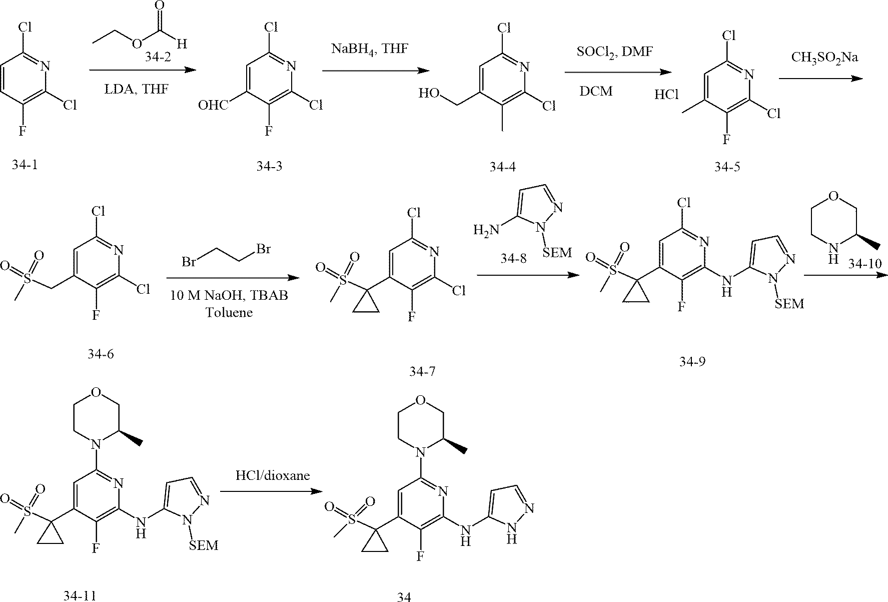

### Step 1. 2,6-dichloro-3-fluoroisonicotinaldehyde (34-3)

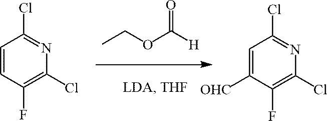

To a solution of 2,6-dichloro-3-fluoropyridine (3 g, 18.07 mmol) in THF (50 mL) at -78° C. was added LDA (2.5 M in THF, 9.4 mL, 23.50 mmol) drop wise. The mixture was stirred at -78° C. for 1 h, then a solution of ethyl formate (2.2 mL, 27.11 mmol) in THF (2 mL) was added drop wise. The mixture was stirred at -78° C. for an additional 1 h. LC-MS showed the reaction was complete. The mixture was quenched with saturated NH₄Cl aqueous solution and extracted with EA (50 mL×3). The combined organic phase was washed with brine, dried over anhydrous Na₂SO₄, filtered and concentrated to dryness. The residue was purified by column chromatography on silica gel (PE : EA = 10:1, V/V) to give the desired product (1.7 g, yield: 48%). ¹H NMR (400 MHz, DMSO) δ 10.11 (s, 1H), 7.91 (d, J= 4.0 Hz, 1H).

### Step 2. (2,6-dichloro-3-fluoropyridin-4-yl)methanol (34-4)

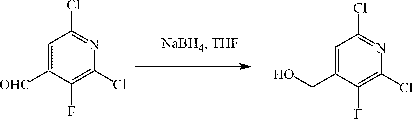

To a solution of 2,6-dichloro-3-fluoroisonicotinaldehyde (1.7 g, 8.76 mmol) in THF (30 mL) at 0° C. was added NaBH₄ (590 mg, 17.53 mmol) portion wise. After the addition, the mixture was stirred at 0° C. for 1 h. LC-MS showed the reaction was complete. The reaction mixture was quenched with saturated NH₄Cl aqueous solution and extracted with EA (40 mL×3). The combined organic phase was washed with brine, dried over anhydrous Na₂SO₄, filtered and concentrated to dryness. The residue was purified by column chromatography on silica gel (PE : EA = 5:1, V/V) to give the desired product (1.62 g, yield: 95%). LC/MS (ESI): m/z 196 [M+H]⁺.

### Step 3. 2,6-dichloro-4-(chloromethyl)-3-fluoropyridine (34-5)

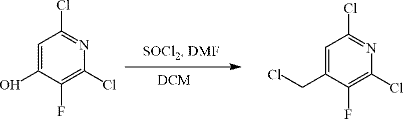

To a solution of (2,6-dichloro-3-fluoropyridin-4-yl)methanol (1.6 g, 8.16 mmol) and DMF (0.05 mL, 0.68 mmol) in DCM (30 mL) at 0° C. was added SOCl₂ (1.2 mL, 16.33 mmol) drop wise. The mixture was stirred at room temperature for 16h. LC-MS showed the reaction was complete. The reaction mixture was concentrated to give the desired product (1.7 g, yield: 97%). LC/MS (ESI) m/z: 214 [M+H]⁺.

### Step 4. 2,6-dichloro-3-fluoro-4-((methylsulfonyl)methyl)pyridine (34-6)

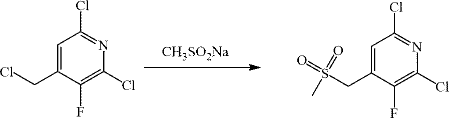

To a solution of 2,6-dichloro-4-(chloromethyl)-3-fluoropyridine (1.7 g, 7.93 mmol) in DMF (30 mL) at 0° C. was added CH₃SO₂Na (1.21 g, 11.89 mmol) portion wise. The mixture was stirred at room temperature for 4 h. LC-MS showed the reaction was complete. The reaction mixture was poured into H₂O (20 mL) and extracted with EA (30 mL×3). The combined organic phase was washed with brine, dried over anhydrous Na₂SO₄, filtered and concentrated to dryness. The residue was purified by flash column chromatography on silica gel (PE:EA = 3:1, V/V) to give the desired product (1.76 g, yield: 86%). LC/MS (ESI): m/z 258 [M+H]⁺.

### Step 5. 2,6-dichloro-3-fluoro-4-(1-(methylsulfonyl)cyclopropyl)pyridine (34-7)

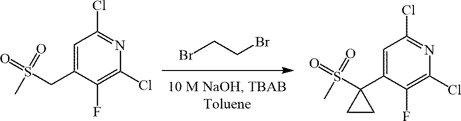

To a solution of 2,6-dichloro-3-fluoro-4-((methylsulfonyl)methyl)pyridine (1.76 g, 6.82 mmol), 1,2-dibromoethane (1.5 mL, 17.05 mmol) and TBAB (440 mg, 1.36 mmol) in Toluene (60 mL) was added NaOH (10 M in H₂O, 6.82 mL, 68.19 mmol). The mixture was stirred at 60° C. for 3 h. LC-MS showed the reaction was complete. The reaction mixture was poured into H₂O (30 mL) and extracted with EA (30 mL×3). The combined organic phase was washed with brine, dried over anhydrous Na₂SO₄, filtered and concentrated. The residue was purified by column chromatography on silica gel (PE : EA = 2:1, V/V) to give the desired product (1.6 g, yield: 83%). LC/MS (ESI): m/z 284 [M+H]⁺.

### Step 6. 6-chloro-3-fluoro-4-(1-(methylsulfonyl)cyclopropyl)-N-(1-((2-(trimethylsilyl) ethoxy)methyl)-1H-pyrazol-5-yl)pyridin-2-amine (34-9)

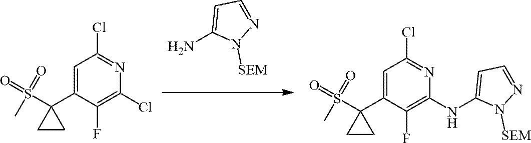 [SMILES: CS(=O)(=O)C1(c2cc(Cl)nc(Cl)c2F)CC1 ; C[Si](C)(C)CCOCn1nccc1N ; C[Si](C)(C)CCOCn1nccc1Nc1nc(Cl)cc(C2(S(C)(=O)=O)CC2)c1F]

To a solution of 2,6-dichloro-3-fluoro-4-(1-(methylsulfonyl)cyclopropyl)pyridine (600 mg, 2.11 mmol), 1-((2-(trimethylsilyl)ethoxy)methyl)-1H-pyrazol-5-amine (450 mg, 2.11 mmol), XantPhos (244 mg, 0.42 mmol) in dioxane (15 mL) were added Pd₂(dba)₃ (193 mg, 0.21 mmol) and Cs₂CO₃ (1.38 g, 4.22 mmol). The mixture was stirred at 100° C. for 6 h under N₂ atmosphere. LC-MS showed the reaction was complete. The reaction mixture was diluted with EA (40 mL), then washed with water and brine, dried over anhydrous Na₂SO₄, filtered and concentrated. The residue was purified by column chromatography on silica gel (PE : EA = 1:1, V/V) to give the desired product (715 mg, yield: 73.5%). LC/MS (ESI): m/z 461 [M+H]⁺.

### Step 7. (R)-3-fluoro-6-(3-methylmorpholino)-4-(1-(methylsulfonyl)cyclopropyl)-N-(1-((2-(trimethylsilyl)ethoxy)methyl)-1H-pyrazol-5-yl)pyridin-2-amine (34-11)

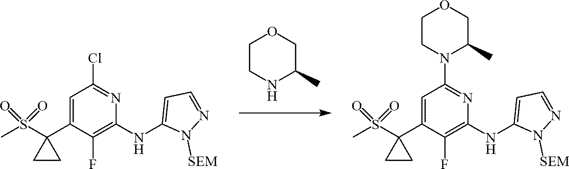

To a solution of 6-chloro-3-fluoro-4-(1-(methylsulfonyl)cyclopropyl)-N-(1-((2-(tri methylsilyl)ethoxy)methyl)-1H-pyrazol-5-yl)pyridin-2-amine (640 mg, 1.39 mmol), (3R)-3-methylmorpholine (281 mg, 2.78 mmol) and RuPhos (130 mg, 0.28 mmol) in dioxane (15 mL) were added Pd₂(dba)₃ (127 mg, 0.14 mmol) and Cs₂CO₃ (1.36 g, 4.16 mmol). The mixture was stirred at 100° C. for 16 h under N₂ atmosphere. LC-MS showed the reaction was complete. The reaction mixture was diluted with EA (40 mL), then washed with water and brine, dried over anhydrous Na₂SO₄, filtered and concentrated. The residue was purified by flash column chromatography on silica gel (DCM : MeOH = 50:1, V/V) to give the desired product (450 mg, yield: 62%). LC/MS (ESI): m/z 527 [M+H]⁺.

### Step 8. (R)-3-fluoro-6-(3-methylmorpholino)-4-(1-(methylsulfonyl)cyclopropyl)-N-(1H-pyrazol-5-yl)pyridin-2-amine (34)

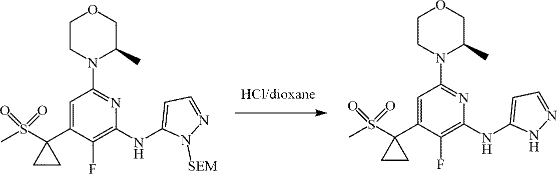

A mixture of (R)-3-fluoro-6-(3-methylmorpholino)-4-(1-(methylsulfonyl)cyclo propyl)-N-(1-((2-(trimethylsilyl)ethoxy)methyl)-1H-pyrazol-5-yl)pyridin-2-amine (450 mg, 0.86 mmol) in HCl solution (4 M in dioxane, 5 mL) was stirred at 60° C. for 1.5 h. LC-MS showed the reaction was complete. The mixture was concentrated under vacuo. The residue was purified by flash column chromatography on silica gel (DCM : MeOH = 30:1, V/V) to afford a pale-yellow oil, which was further purified by Prep-HPLC (C₁₈, 10-95%, MeOH in H₂O with 0.1% HCOOH) to give the desired product (45 mg, yield: 13%). LC/MS (ESI): m/z 396 [M+H]⁺. ¹H NMR (400 MHz, DMSO) δ 12.17 (s, 1H), 8.80 (s, 1H), 7.58 (s, 1H), 6.47 (s, 1H), 6.11 (s, 1H), 4.22 - 4.15 (m, 1H), 3.93 (dd, J = 11.2, 3.1 Hz, 1H), 3.74 -3.66 (m, 2H), 3.61 (dd, J = 11.3, 2.7 Hz, 1H), 3.48 - 3.46 (m, 1H), 3.04 (dd, J = 12.5, 3.7 Hz, 1H), 2.98 (s, 3H), 1.70 - 1.62 (m, 2H), 1.37 - 1.30 (m, 2H), 1.11 (d, J = 6.6 Hz, 3H).

### Example 29

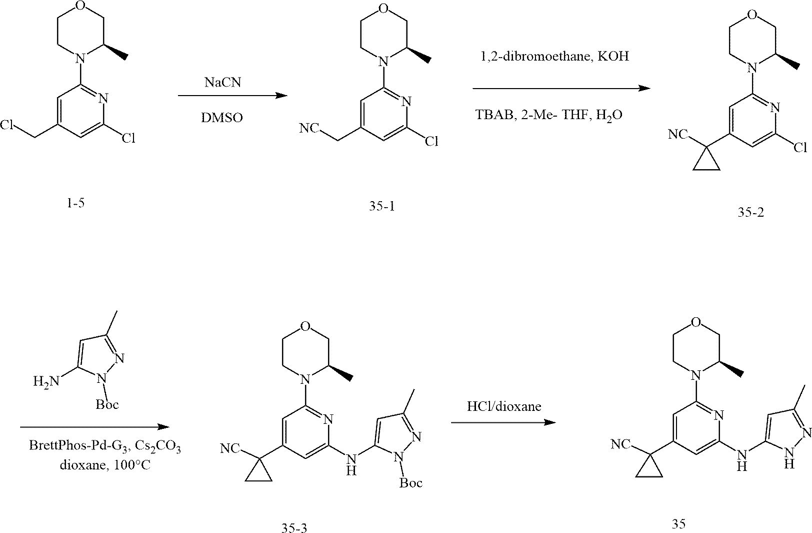

### Step 1. 2-{2-chloro-6-[(3R)-3-methylmorpholin-4-yl]pyridin-4-yl}acetonitrile (35-1)

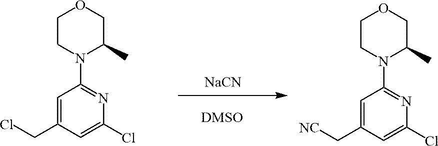

To a solution of (3R)-4-[6-chloro-4-(chloromethyl)pyridin-2-yl]-3-methylmorpholine (778 mg, 2.98 mmol) in DMSO (15 mL) was added NaCN (219 mg, 4.47 mmol) portion wise. The mixture was stirred at ambient temperature overnight. LC-MS showed the reaction was complete. The mixture was diluted with EA (50 mL), then washed with water and brine, dried over anhydrous Na₂SO₄, filtered and concentrated. The residue was purified by column chromatography on silica gel (PE : EA = 3:1, V/V) to give the desired product (253 mg, yield: 34%). LC/MS ESI (m/z): 252 [M+H]⁺.

### Step 2. 1-{2-chloro-6-[(3R)-3-methylmorpholin-4-yl]pyridin-4-yl}cyclopropane-1-carbonitrile (35-2)

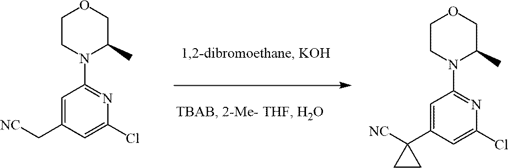

A mixture of 2-{2-chloro-6-[(3R)-3-methylmorpholin-4-yl]pyridin-4-yl}acetonitrile (80 mg, 0.32 mmol), 1,2-dibromoethane (0.06 mL, 0.64 mmol), KOH (500 mg, 8.91 mmol) and TBAB (21 mg, 0.06 mmol) in 2-Methyltetrahydrofuran (2 mL) and H₂O (0.5 mL) was stirred at 60° C. overnight under nitrogen atmosphere. LC-MS showed the reaction was complete. The mixture was diluted with EA (50 mL), then washed with water and brine, dried over anhydrous Na₂SO₄, filtered and concentrated. The residue was purified by column chromatography on silica gel (PE : EA = 2:1, V/V) to give the desired product (64 mg, yield: 73%). LC/MS ESI (m/z): 278 [M+H]⁺.

### Step 3. Tert-butyl 5-{[4-(1-cyanocyclopropyl)-6-[(3R)-3-methylmorpholin-4-yl] pyridin-2-yl]amino}-3-methyl-1H-pyrazole-1-carboxylate (35-3)

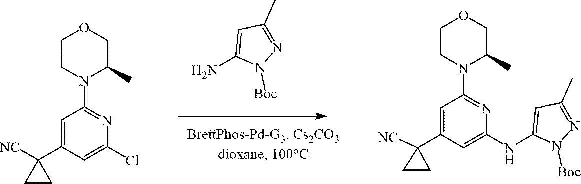

A mixture of 1-{2-chloro-6-[(3R)-3-methylmorpholin-4-yl]pyridin-4-yl}cyclo propane-1-carbonitrile (64 mg, 0.23 mmol), tert-butyl 5-amino-3-methyl-1H-pyrazole-1-carboxylate (68 mg, 0.35 mmol), BrettPhos-Pd-G3 (20 mg, 0.02 mmol) and Cs₂CO₃ (225 mg, 0.69 mmol) in dioxane (6 mL) was stirred at 100° C. overnight under nitrogen atmosphere. LC-MS showed the reaction was complete. The mixture was diluted with EA (50 mL), then washed with water and brine, dried over anhydrous Na₂SO₄, filtered and concentrated. The residue was purified by column chromatography on silica gel (PE : EA = 1:1, V/V) to give the desired product (62 mg, yield: 61%). LC/MS ESI (m/z): 439 [M+H]⁺.

### Step 4. 1-{2-[(3-methyl-1H-pyrazol-5-yl)amino]-6-[(3R)-3-methylmorpholin-4-yl]pyridin-4-yl}cyclopropane-1-carbonitrile (35)

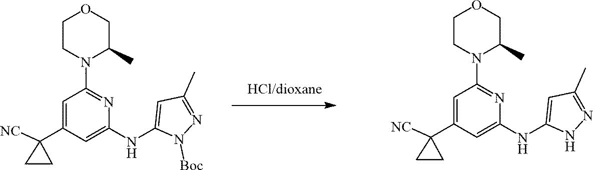

A solution of tert-butyl 5-{[4-(1-cyanocyclopropyl)-6-[(3R)-3-methylmorpholin-4-yl] pyridin-2-yl]amino}-3-methyl-1H-pyrazole-1-carboxylate (62 mg, 0.14 mmol) in HCl solution (4 M in dioxane, 4 mL) was stirred at ambient temperature overnight. LC-MS showed the reaction was complete. The mixture was concentrated under reduced pressure to dryness. The residue was purified by prep-HPLC (C₁₈, 10-95%, MeOH in H₂O with 0.1% HCOOH) to give the desired product (15.4 mg, yield: 32%). LC/MS ESI (m/z): 339 [M+H]⁺. ¹H NMR (400 MHz, DMSO) δ 11.72 (br, 1H), 8.81 (s, 1H), 6.57 (s, 1H), 5.99 (s, 1H), 5.77 (s, 1H), 4.30 (d, J = 6.5 Hz, 1H), 3.92 (dd, J = 11.2, 3.2 Hz, 1H), 3.76 (d, J = 12.7 Hz, 1H), 3.71 (d, J = 11.2 Hz, 1H), 3.60 (dd, J = 11.2, 2.8 Hz, 1H), 3.47 - 3.44 (m, 1H), 3.03 (dd, J = 12.6, 9.0 Hz, 1H), 2.17 (s, 3H), 1.71 (dd, J = 7.5, 4.4 Hz, 2H), 1.50 (dd, J = 7.7, 4.4 Hz, 2H), 1.11 (d, J = 6.6 Hz, 3H).

### Example 30

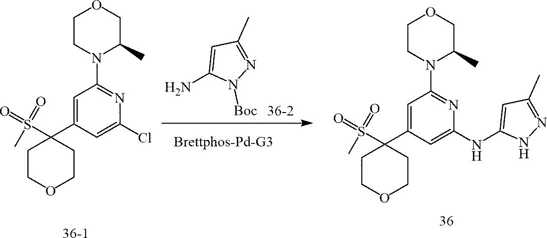

### Step 1. (R)-N-(3-methyl-1H-pyrazol-5-yl)-6-(3-methylmorpholino)-4-(4-(methyl sulfonyl)tetrahydro-2H-pyran-4-yl)pyridin-2-amine (36)

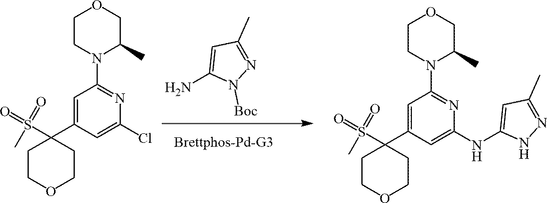

To a solution of (R)-4-(6-chloro-4-(4-(methylsulfonyl)tetrahydro-2H-pyran-4-yl) pyridin-2-yl)-3-methylmorpholine (100 mg, 0.27 mmol), tert-butyl 5-amino-3-methyl-1H-pyrazole-1-carboxylate (105 mg, 0.53 mmol) and Cs₂CO₃ (261 mg, 0.80 mmol) in dioxane (8 mL) was added BrettPhos-Pd-G3 (24 mg, 0.027 mmol). The mixture was stirred at 100° C. for 16 h under N₂ atmosphere. LC-MS showed the reaction was complete. The reaction mixture was diluted with EA (60 mL), then washed with water and brine, dried over anhydrous Na₂SO₄, filtered and concentrated to dryness. The residue was purified by Prep-HPLC (C₁₈, 10-95%, MeOH in H₂O with 0.1% HCOOH) to give the desired product (12 mg, yield: 10 %). LC/MS (ESI): m/z 436 [M+H]⁺. ¹H NMR (400 MHz, DMSO) δ 11.69 (s, 1H), 8.82 (s, 1H), 6.70 (s, 1H), 6.17 (s, 1H), 6.05 (s, 1H), 4.31 - 4.23 (m, 1H), 3.963.83 (m, 4H), 3.72 (d, J = 11.2 Hz, 1H), 3.64 (dd, J = 11.2, 2.7 Hz, 1H), 3.49 (td, J = 11.7, 2.8 Hz, 1H), 3.26 -3.18 (m, 2H), 3.05 (td, J = 12.7, 3.6 Hz, 1H), 2.71 (s, 3H), 2.43 (d, J = 13.7 Hz, 2H), 2.24 - 2.16 (m, 5H), 1.12 (d, J = 6.6 Hz, 3H).

### Example 31

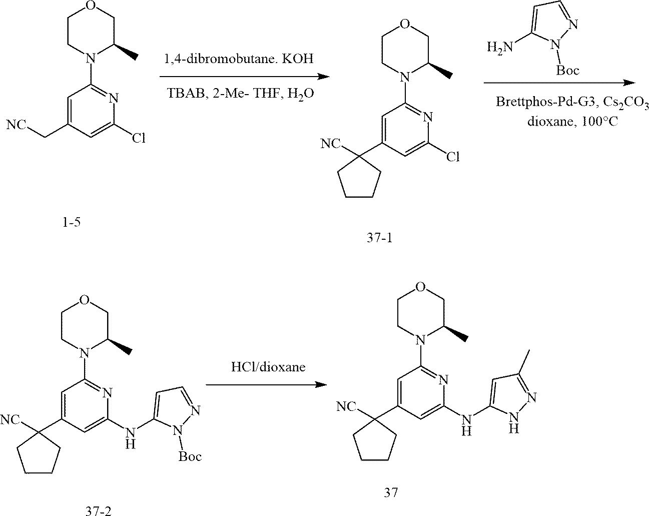

### Step 1. 1-{2-chloro-6-[(3R)-3-methylmorpholin-4-yl]pyridin-4-yl}cyclopentane-1-carbonitrile (37-1)

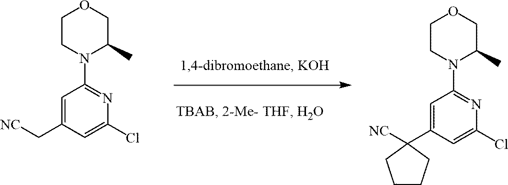

A mixture of 2-{2-chloro-6-[(3R)-3-methylmorpholin-4-yl]pyridin-4-yl}acetonitrile (210 mg, 0.83 mmol), 1,4-dibromobutane (1 mL, 8.34 mmol), KOH (3 g, 53.47 mmol) and TBAB (54 mg, 0.17 mmol) in co-solvent of 2-methyltetrahydrofuran (15 mL) and H₂O (3 mL) was stirred at 70° C. overnight. LC-MS showed the reaction was complete. The mixture was diluted with EA (50 mL), then washed with water and brine, dried over anhydrous Na₂SO₄, filtered and concentrated. The residue was purified by column chromatography on silica gel (PE : EA = 3:1, V/V) to give the desired product (192 mg, yield: 75%). LC/MS ESI (m/z): 306 [M+H]⁺.

### Step 2. Tert-butyl 5-{[4-(1-cyanocyclopentyl)-6-[(3R)-3-methylmorpholin-4-yl] pyridin-2-yl]amino}-1H-pyrazole-1-carboxylate (37-2)

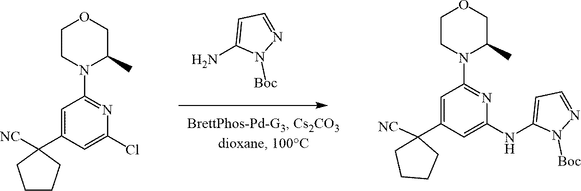

To a solution of 1-{2-chloro-6-[(3R)-3-methylmorpholin-4-yl]pyridin-4-yl}cyclo pentane-1-carbonitrile (100 mg, 0.33 mmol) and tert-butyl 5-amino-1H-pyrazole-1-carboxylate (90 mg, 0.49 mmol) in dioxane (10 mL) were added BrettPhos-Pd-G3 (29.6 mg, 0.03 mmol) and Cs₂CO₃ (319.6 mg, 0.98 mmol). The mixture was stirred at 100° C. overnight under nitrogen atmosphere. LC-MS showed the reaction was complete. The mixture was diluted with EA (50 mL), then washed with water and brine, dried over anhydrous Na₂SO₄, filtered and concentrated. The residue was purified by column chromatography on silica gel (PE : EA = 1:1, V/V) to give the desired product (104 mg, yield: 70%). LC/MS ESI (m/z): 453 [M+H]⁺.

### Step 3. 1-{2-[(3R)-3-methylmorpholin-4-yl]-6-[(1H-pyrazol-5-yl)amino]pyridin-4-yl}cyclopentane-1-carbonitrile (37)

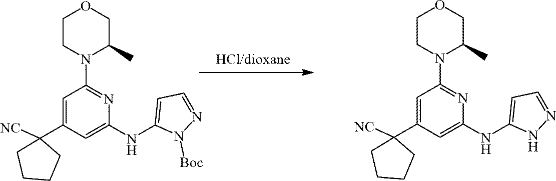

A mixture of tert-butyl 5-{[4-(1-cyanocyclopentyl)-6-[(3R)-3-methylmorpholin-4-yl]pyridin-2-yl]amino}-1H-pyrazole-1-carboxylate (104 mg, 0.23 mmol ) in HCl solution (4 M in dioxane, 4 mL) was stirred at ambient temperature overnight. LC-MS showed the reaction was complete. The mixture was concentrated under reduced pressure to dryness. The residue was purified by prep-HPLC (C₁₈, 10-95%, MeOH in H₂O with 0.1% HCOOH) to give the desired product (42.5 mg, yield: 52%). LC/MS ESI (m/z): 353 [M+H]⁺. ¹H NMR (400 MHz, DMSO) δ 12.14 (s, 1H), 9.01 (s, 1H), 7.54 (d, J = 2.2 Hz, 1H), 6.59 (s, 1H), 6.30 (d, J = 2.2 Hz, 1H), 6.07 (d, J = 0.9 Hz, 1H), 4.35 - 4.29 (m, 1H), 3.93 (dd, J = 11.2, 3.3 Hz, 1H), 3.79 (d, J = 13.0 Hz, 1H), 3.73 (d, J = 11.2 Hz, 1H), 3.62 (dd, J = 11.3, 2.8 Hz, 1H), 3.48 (dd, J = 11.8, 2.9 Hz, 1H), 3.09 - 3.02 (m, 1H), 2.34 - 2.28 (m, 2H), 2.10 - 2.03 (m, 2H), 1.90 -1.85 (m, 4H), 1.13 (d, J = 6.6 Hz, 3H).

### Example 32

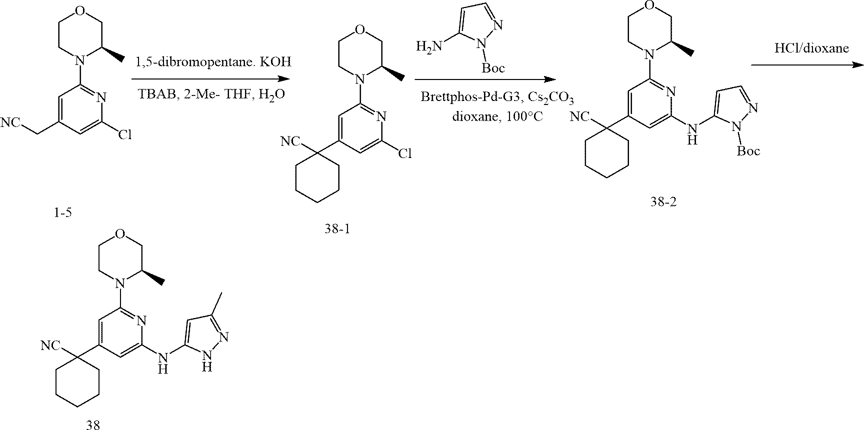

### Step 1. 1-{2-chloro-6-[(3R)-3-methylmorpholin-4-yl]pyridin-4-yl}cyclohexane-1-carbonitrile (38-1)

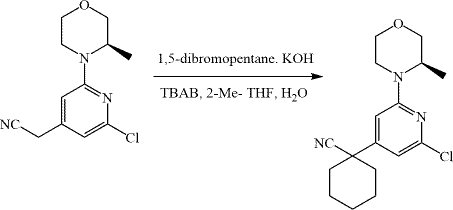

A mixture of 2-{2-chloro-6-[(3R)-3-methylmorpholin-4-yl]pyridin-4-yl}acetonitrile (210 mg, 0.83 mmol), 1,5-dibromopentane (1.2 mL, 8.34 mmol), KOH (3 g, 53.47 mmol) and TBAB (54 mg, 0.17 mmol) in co-solvent of 2-methyltetrahydrofuran (15 mL) and H₂O (3 mL) was stirred at 70° C. overnight. LC-MS showed the reaction was complete. The mixture was diluted with EA (50 mL), then washed with water and brine, dried over anhydrous Na₂SO₄, filtered and concentrated. The residue was purified by column chromatography on silica gel (PE : EA = 3:1, V/V) to give the desired product (194 mg, yield: 73%). LC/MS ESI (m/z): 320 [M+H]⁺.

### Step 2. Tert-butyl 5-{[4-(1-cyanocyclohexyl)-6-[(3R)-3-methylmorpholin-4-yl] pyridin-2-yl]amino}-1H-pyrazole-1-carboxylate (38-2)

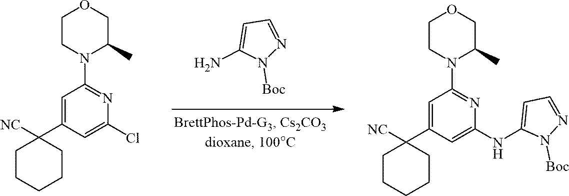

To a solution of 1-{2-chloro-6-[(3R)-3-methylmorpholin-4-yl]pyridin-4-yl}cyclohexane-1-carbonitrile (100 mg, 0.31 mmol) and tert-butyl 5-amino-1H-pyrazole-1-carboxylate (86 mg, 0.47 mmol) in dioxane (10 mL) were added BrettPhos-Pd-G3 (28 mg, 0.03 mmol) and Cs₂CO₃ (306 mg, 0.94 mmol). The mixture was stirred at 100° C. overnight under nitrogen atmosphere. LC-MS showed the reaction was complete. The mixture was diluted with EA (50 mL), then washed with water and brine, dried over anhydrous Na₂SO₄, filtered and concentrated. The residue was purified by column chromatography on silica gel (PE : EA = 1:1, V/V) to give the desired product (102 mg, yield: 70%). LC/MS ESI (m/z): 467 [M+H]⁺.

### Step 3. 1-{2-[(3R)-3-methylmorpholin-4-yl]-6-[(1H-pyrazol-5-yl)amino]pyridin-4-yl} cyclohexane-1-carbonitrile (38)

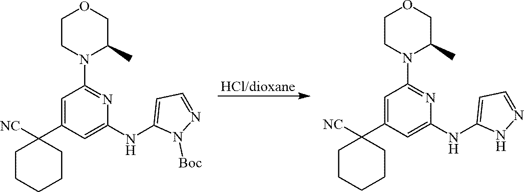

A mixture of tert-butyl 5-{[4-(1-cyanocyclohexyl)-6-[(3R)-3-methylmorpholin-4-yl] pyridin-2-yl]amino}-1H-pyrazole-1-carboxylate (102 mg, 0.22 mmol) in HCl solution (4 M in dioxane, 4 mL) was stirred at ambient temperature overnight. LC-MS showed the reaction was complete. The mixture was concentrated under reduced pressure to dryness. The residue was purified by prep-HPLC (C₁₈, 10-95%, MeOH in H₂O with 0.1% HCOOH) to give the desired product (36.6 mg, yield: 46%). LC/MS ESI (m/z): 367 [M+H]⁺. ¹H NMR (400 MHz, DMSO) δ 12.17 (s, 1H), 9.00 (s, 1H), 7.53 (d, J = 2.2 Hz, 1H), 6.63 (s, 1H), 6.29 (d, J = 2.1 Hz, 1H), 6.11 (d, J = 0.8 Hz, 1H), 4.32 (d, J = 6.6 Hz, 1H), 3.93 (dd, J = 11.2, 3.3 Hz, 1H), 3.80 (d, J = 11.3 Hz, 1H), 3.73 (d, J = 11.1 Hz, 1H), 3.62 (dd, J = 11.3, 2.8 Hz, 1H), 3.47 (td, J = 11.8, 3.0 Hz, 1H), 3.05 (td,J = 12.6, 3.7 Hz, 1H), 2.00 (d, J = 12.4 Hz, 2H), 1.81 (dd, J = 16.4, 6.6 Hz, 4H), 1.73 (d, J = 13.6 Hz, 1H), 1.66 - 1.56 (m, 2H), 1.34 - 1.24 (m, 1H), 1.12 (d, J = 6.6 Hz, 3H).

### Example 33

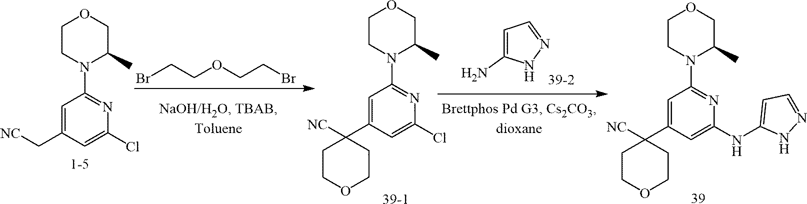

### Step 1. (R)-4-(2-chloro-6-(3-methylmorpholino)pyridin-4-yl)tetrahydro-2H-pyran-4-carbonitrile (39-1)

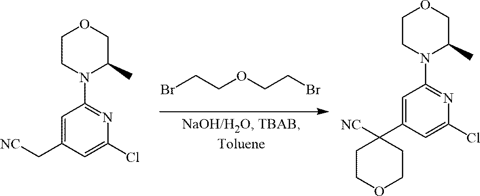

A mixture of 2-{2-chloro-6-[(3R)-3-methylmorpholin-4-yl]pyridin-4-yl}acetonitrile (180 mg, 0.72 mmol), 1-bromo-2-(2-bromoethoxy)ethane (660 mg, 2.85 mmol), TBAB (46 mg, 0.14 mmol) and NaOH (10.0 M in H₂O, 14.0 mmol, 1.4 mL) in toluene (10 mL) was stirred at 60° C. for 2 h. LC-MS showed the reaction was complete. The reaction mixture was diluted with EA (40 mL), then washed with water and brine, dried over anhydrous Na₂SO₄, filtered and concentrated under vacuo. The residue was purified by column chromatography on silica gel (PE : EA = 2: 1, V/V) to afford the desired product (157 mg, yield: 68%). LC/MS (ESI): m/z 322 [M+H]⁺.

### Step 2. (R)-4-(2-((1H-pyrazol-5-yl)amino)-6-(3-methylmorpholino)pyridin-4-yl) tetrahydro-2H-pyran-4-carbonitrile (39)

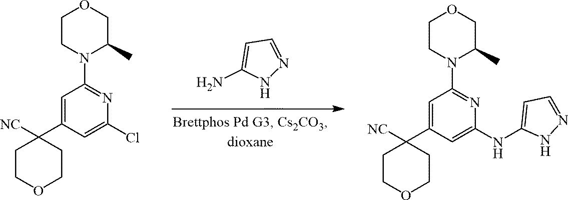

A mixture of 4-{2-chloro-6-[(3R)-3-methylmorpholin-4-yl]pyridin-4-yl}oxane-4-carbonitrile (80 mg, 0.25 mmol), 1H-pyrazol-5-amine (41 mg, 0.49 mmol), BrettPhos-Pd-G3 (22 mg, 0.02 mmol) and Cs₂CO₃ (244 mg, 0.75 mmol) in dioxane (4 mL) was stirred at 110° C. for 16 h under N₂ atmosphere. LC-MS showed the reaction was complete. The reaction mixture was diluted with EA (40 mL), then washed with water and brine, dried over anhydrous Na₂SO₄, filtered and concentrated. The residue was purified by Prep-HPLC (C₁₈, 10-95%, MeOH in H₂O with 0.1% HCOOH) to give the desired product (37 mg, yield: 40%). LC/MS (ESI): m/z 369 [M+H]⁺. ¹H NMR (400 MHz, DMSO) δ 12.06 (s, 1H), 9.04 (s, 1H), 7.54 (d, J = 1.9 Hz, 1H), 6.64 (s, 1H), 6.29 (s, 1H), 6.13 (s, 1H), 4.34 (d, J = 6.9 Hz, 1H), 4.00 (dd, J = 11.2, 3.0 Hz, 2H), 3.93 (dd, J = 11.3, 3.3 Hz, 1H), 3.82 (d, J = 12.8 Hz, 1H), 3.73 (d, J = 11.2 Hz, 1H), 3.68 - 3.59 (m, 3H), 3.47 (td, J = 11.8, 2.9 Hz, 1H), 3.06 (td, J = 12.6, 3.7 Hz, 1H), 2.13 - 1.95 (m, 4H), 1.13 (d, J = 6.6 Hz, 3H).

### Example 34

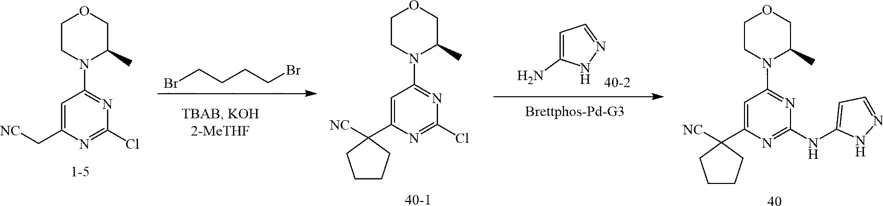

### Step 1. (R)-1-(2-chloro-6-(3-methylmorpholino)pyrimidin-4-yl)cyclopentane-1-carbonitrile (40-1)

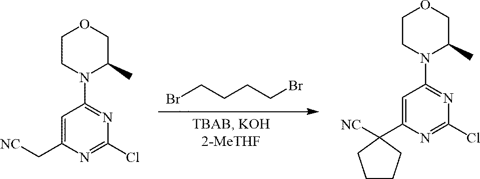

To a solution of (R)-2-(2-chloro-6-(3-methylmorpholino)pyrimidin-4-yl)acetonitrile (200 mg, 0.79 mmol), 1,4-dibromobutane (0.95 mL, 7.91 mmol) and TBAB (26 mg, 0.08 mmol) in 2-MeTHF (15 mL) was added KOH aqueous solution (10 M, 1.58 mL, 15.8 mmol). The mixture was stirred at 70° C. overnight. LC-MS showed the reaction was complete. The reaction mixture was diluted with EA (60 mL), then washed with water and brine, dried over anhydrous Na₂SO₄, filtered and concentrated to dryness. The residue was purified by column chromatography on silica gel (PE : EA = 10:1, V/V) to give the desired product (220 mg, yield: 91%). LC/MS (ESI): m/z 307 [M+H]⁺.

### Step 2. (R)-1-(2-((1H-pyrazol-5-yl)amino)-6-(3-methylmorpholino)pyrimidin-4-yl) cyclopentane-1-carbonitrile (40)

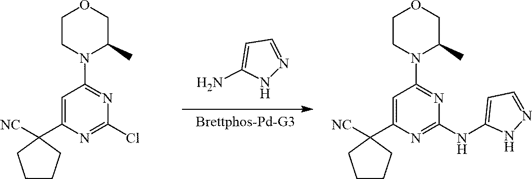

To a suspension of (R)-1-(2-chloro-6-(3-methylmorpholino)pyrimidin-4-yl)cyclo pentane-1-carbonitrile (100 mg, 0.33 mmol), 1H-pyrazol-5-amine (41 mg, 0.49 mmol) and Cs₂CO₃ (319 mg, 0.98 mmol) in dioxane (8 mL) was added BrettPhos-Pd-G3 (29 mg, 0.03 mmol). The mixture was stirred at 100° C. for 16 h. LC-MS showed the reaction was complete. The reaction mixture was diluted with DCM (50 mL), then washed with water and brine, dried over anhydrous Na₂SO₄, filtered and concentrated to dryness. The residue was purified by Prep-HPLC (C₁₈, 10-95%, MeOH in H₂O with 0.1% HCOOH) to give the desired product (30 mg, yield: 26%). LC/MS (ESI): m/z 354.4 [M+H]⁺. ¹H NMR (400 MHz, DMSO) δ 12.09 (s, 1H), 9.19 (s, 1H), 7.52 (s, 1H), 6.49 (s, 1H), 6.28 (s, 1H), 4.40 (d, J = 5.4 Hz, 1H), 4.00 (d, J = 12.9 Hz, 1H), 3.93 (dd, J = 11.3, 3.3 Hz, 1H), 3.72 (d, J = 11.4 Hz, 1H), 3.59 (dd, J = 11.4, 2.9 Hz, 1H), 3.46 - 3.40 (m, 1H), 3.14 (td, J = 12.9, 3.7 Hz, 1H), 2.31 - 2.22 (m, 4H), 1.89 - 1.82 (m, 4H), 1.19 (d, J = 6.7 Hz, 3H).

### Example 35

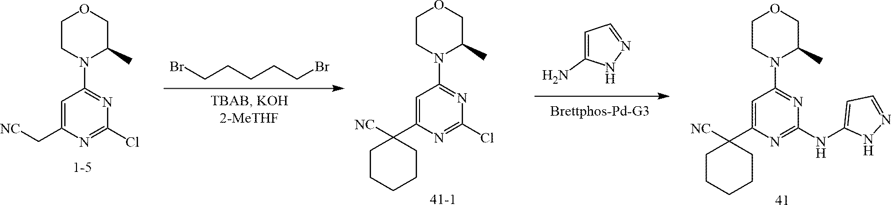

### Step 1. (R)-1-(2-chloro-6-(3-methylmorpholino)pyrimidin-4-yl)cyclohexane-1-carbonitrile (41-1)

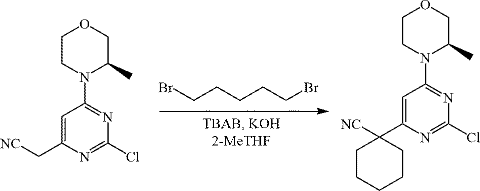

To a suspension of (R)-2-(2-chloro-6-(3-methylmorpholino)pyrimidin-4-yl) acetonitrile (200 mg, 0.79 mmol), 1,5-dibromopentane (1.08 mL, 7.91 mmol) and TBAB (26 mg, 0.08 mmol) in 2-MeTHF (15 mL) was added KOH aqueous solution (10 M, 1.58 mL, 15.8 mmol). The mixture was stirred at 70° C. for 16 h. LC-MS showed the reaction was complete. The reaction mixture was diluted with EA (60 mL), then washed with water and brine, dried over anhydrous Na₂SO₄, filtered and concentrated to dryness. The residue was purified by column chromatography on silica gel (PE : EA = 10:1, V/V) to give the desired product (210 mg, yield: 83%). LC/MS (ESI): m/z 321 [M+H]⁺.

### Step 2. (R)-1-(2-((1H-pyrazol-5-yl)amino)-6-(3-methylmorpholino)pyrimidin-4-yl)cyclohexane-1-carbonitrile (41)

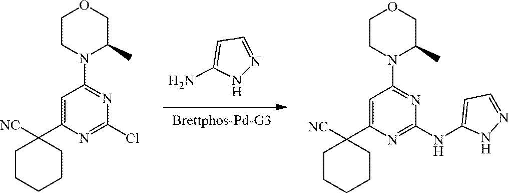

To a suspension of (R)-1-(2-chloro-6-(3-methylmorpholino)pyrimidin-4-yl)cyclo hexane-1-carbonitrile (100 mg, 0.31 mmol), 1H-pyrazol-5-amine (39 mg, 0.47 mmol) and Cs₂CO₃ (305 mg, 0.94 mmol) in dioxane (8 mL) was added BrettPhos-Pd-G3 (28 mg, 0.03 mmol). The mixture was stirred at 100° C. for 16 h. LC-MS showed the reaction was complete. The reaction mixture was diluted with EA (50 mL), then washed with water and brine, dried over anhydrous Na₂SO₄, filtered and concentrated to dryness. The residue was purified by Prep-HPLC (C₁₈, 10-95%, MeOH in H₂O with 0.1% HCOOH) to give the desired product (20 mg, yield: 17.5%). LC/MS (ESI): m/z 368.5 [M+H]⁺. ¹H NMR (400 MHz, DMSO) δ 12.09 (s, 1H), 9.18 (s, 1H), 7.51 (s, 1H), 6.52 (s, 1H), 6.26 (s, 1H), 4.46 - 4.34 (m, 1H), 4.01 (d, J = 13.3 Hz, 1H), 3.93 (dd, J = 11.4, 3.3 Hz, 1H), 3.72 (d, J = 11.4 Hz, 1H), 3.58 (dd, J = 11.4, 3.0 Hz, 1H), 3.43 (td, J = 11.9, 2.9 Hz, 1H), 3.14 (td, J = 12.8, 3.6 Hz, 1H), 2.09 -2.02 (m, 2H), 1.95 - 1.86 (m, 2H), 1.85 - 1.78 (m, 2H), 1.77 - 1.70 (m, 1H), 1.65 -1.53 (m, 2H), 1.32 - 1.22 (m, 1H), 1.19 (d, J = 6.7 Hz, 3H).

### Example 36

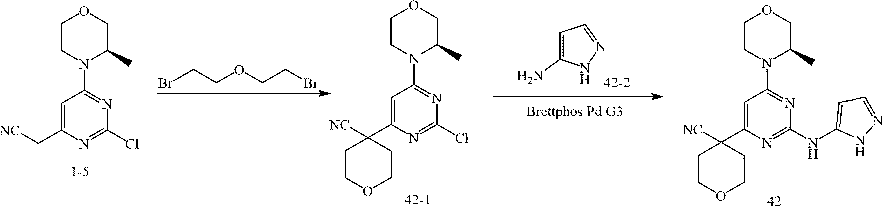

### Step 1. (R)-4-(2-chloro-6-(3-methylmorpholino)pyrimidin-4-yl)tetrahydro-2H-pyran-4-carbonitrile (42-1)

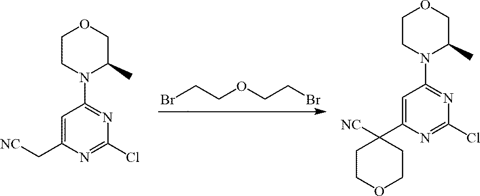

To a solution of (R)-2-(2-chloro-6-(3-methylmorpholino)pyrimidin-4-yl)acetonitrile (200 mg, 0.79 mmol) in Toluene (10 mL) were added 1-bromo-2-(2-bromo ethoxy)ethane (367 mg, 1.58 mmol), Sodium hydroxide (10 M in H₂O, 0.79 mL, 7.91 mmol) and TBAB (52 mg, 0.16 mmol). The reaction was stirred at 60° C. overnight. LC-MS showed the reaction was complete. The mixture was diluted with EA (60 mL), then washed with water and brine, dried over anhydrous Na₂SO₄, filtered and concentrated. The residue was purified by flash chromatography on silica gel (PE : EA = 3:1, V/V) to afford the desired product (180 mg, yield: 70%). LC/MS (ESI): m/z 323 [M+H]⁺.

### Step 2. (R)-4-(2-((1H-pyrazol-5-yl)amino)-6-(3-methylmorpholino)pyrimidin-4-yl) tetrahydro-2H-pyran-4-carbonitrile (42)

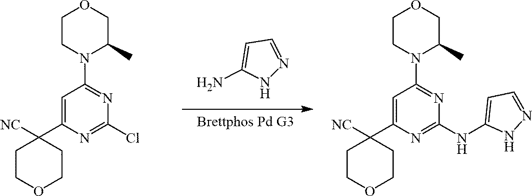

To a solution of (R)-4-(2-chloro-6-(3-methylmorpholino)pyrimidin-4-yl)tetrahydro -2H-pyran-4-carbonitrile (80 mg, 0.25 mmol) in dioxane (8 mL) were added 1H-pyrazol-5-amine (31 mg, 0.37 mmol), Cs₂CO₃ (162 mg, 0.50 mmol) and BrettPhos-Pd-G3 (45 mg, 0.05 mmol). The mixture was stirred at 100° C. overnight under nitrogen atmosphere. LC-MS showed the reaction was complete. The mixture was diluted with EA (60 mL), then washed with water and brine, dried over anhydrous Na₂SO₄, filtered and concentrated. The residue was purified by Prep -HPLC (C18, 10-95%, MeOH in H₂O with 0.1% HCOOH) to afford the desired product (15 mg, yield: 16%). LC/MS (ESI): m/z 370 [M+H]⁺. ¹HNMR (400 MHz, DMSO) δ 12.10 (s, 1H), 9.14 (s, 1H), 7.54 (s, 1H), 6.60 (s, 1H), 6.29 (s, 1H), 4.43 (s, 1H), 4.07 - 3.96 (m, 3H), 3.93 (dd, J = 11.4, 3.4 Hz, 1H), 3.72 (d, J = 11.4 Hz, 1H), 3.63 (dd, J = 12.0, 10.3 Hz, 2H), 3.57 (d, J = 3.0 Hz, 1H), 3.43 (td, J = 11.9, 2.9 Hz, 1H), 3.15 (td, J = 12.9, 3.6 Hz, 1H), 2.20 - 2.12 (m, 2H), 2.03 (d, J = 12.3 Hz, 2H), 1.20 (d, J = 6.7 Hz, 3H).

### Example 37

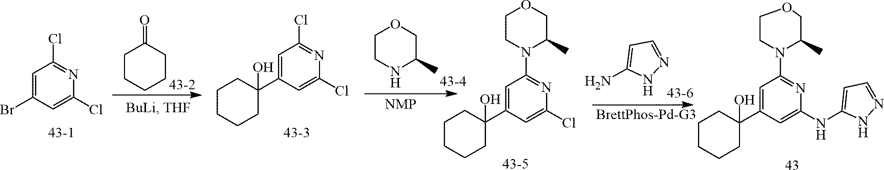

### Step 1. 1-(2,6-dichloropyridin-4-yl)cyclohexan-1-ol (43-3)

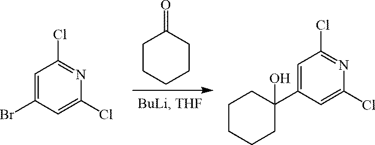

To a solution of 4-bromo-2,6-dichloropyridine (300 mg, 1.32 mmol) and cyclo- hexanone (156 mg, 1.59 mmol) in THF (8 mL) at -78° C. was added n-BuLi solution (2.5 M in THF, 0.74 mL, 1.85 mmol) drop wise. The mixture was stirred at -78° C. for 1 h. LC-MS showed the reaction was complete. The mixture was quenched with saturated NH₄Cl aqueous solution and extracted with EA (30 mL×3) thrice. The combined organic phase was washed with brine, dried over anhydrous Na₂SO₄, filtered and concentrated to dryness. The residue was purified by column chromatography on silica gel (PE : EA= 10:1, V/V) to give the desired product (252 mg, yield: 77%). LC/MS (ESI): m/z 246 [M+H]⁺.

### Step 2. (R)-1-(2-chloro-6-(3-methylmorpholino)pyridin-4-yl)cyclohexan-1-ol (43-5)

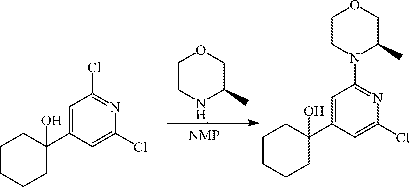

To a solution of 1-(2,6-dichloropyridin-4-yl)cyclohexan-1-ol (250 mg, 1.02 mmol) in NMP (5 mL) was added (3R)-3-methylmorpholine (308 mg, 3.05 mmol). The mixture was stirred at 150° C. for 16 h. LC-MS showed the reaction was complete. The reaction mixture was diluted with EA (60 mL), then washed with water and brine, dried over anhydrous Na₂SO₄, filtered and concentrated to dryness. The residue was purified by column chromatography on silica gel (PE : EA = 5:1, V/V) to give the desired product (42 mg, yield: 13.3%). LC/MS (ESI): m/z 311 [M+H]⁺.

### Step 3. (R)-1-(2-((1H-pyrazol-5-yl)amino)-6-(3-methylmorpholino)pyridin-4-yl) cyclohexan-1-ol (43)

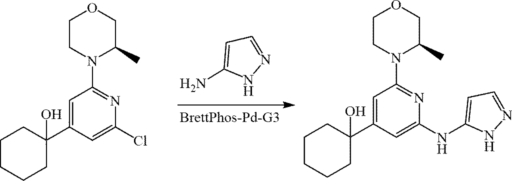

To a suspension of (R)-1-(2-chloro-6-(3-methylmorpholino)pyridin-4-yl)cyclohexan-1-ol (42 mg, 0.135 mmol), 1H-pyrazol-5-amine (23 mg, 0.270 mmol) and Cs₂CO₃ (132 mg, 0.405 mmol) in dioxane (6 mL) was added BrettPhos-Pd-G3 (12 mg, 0.01 mmol). The mixture was stirred at 100° C. for 16 h under N₂ atmosphere. LC-MS showed the reaction was complete. The reaction mixture was diluted with EA (40 mL), then washed with water and brine, dried over anhydrous Na₂SO₄, filtered and concentrated to dryness. The residue was purified by Prep-HPLC (C₁₈, 10-95%, MeOH in H₂O with 0.1% HCOOH) to give the desired product (10 mg, yield: 21%). LC/MS (ESI): m/z 358 [M+H]⁺. ¹H NMR (400 MHz, DMSO) δ 12.17 (s, 1H), 8.74 (s, 1H), 7.50 (d, J = 2.2 Hz, 1H), 6.54 (s, 1H), 6.28 (d, J = 2.0 Hz, 1H), 6.15 (s, 1H), 4.60 (s, 1H), 4.32 - 4.23 (m, 1H), 3.93 (dd, J = 11.1, 3.1 Hz, 1H), 3.77 - 3.68 (m, 2H), 3.62 (dd, J = 11.2, 2.8 Hz, 1H), 3.47 (td, J = 11.7, 2.9 Hz, 1H), 3.03 (td, J = 12.5, 3.6 Hz, 1H), 1.73 - 1.53 (m, 7H), 1.51 - 1.43 (m, 2H), 1.27 - 1.17 (m, 1H), 1.11 (d, J = 6.6 Hz, 3H).

### Example 38

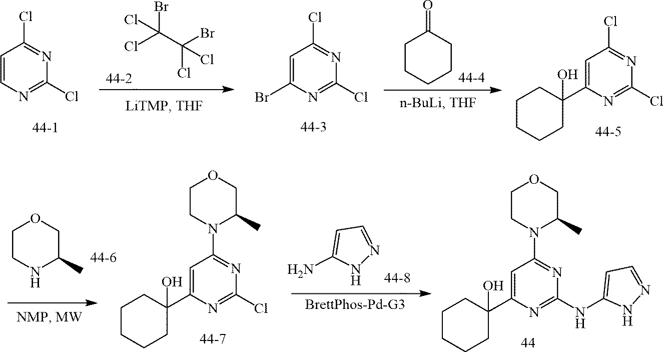

### Step 1. 4-bromo-2,6-dichloropyrimidine (44-3)

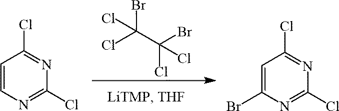

To a solution of 2,4-dichloropyrimidine (1 g, 6.71 mmol) in THF (50 mL) at -60° C. was added Lithium tetramethylpiperidide solution (1.0 M in THF, 8.0 mL, 8.05 mmol) dropwise. The mixture was stirred at -60° C. for 1 h, then a solution of 1,2-dibromo-1,1,2,2-tetrachloroethane (3.28 g, 10.07 mmol) in THF (5 mL) was added dropwise. The resulting mixture was stirred at -60° C. for an additional 2 h. LC-MS showed the reaction was complete. The reaction mixture was quenched with saturated NH₄Cl aqueous solution and extracted with EA (60 mL). The organic layer was separated, then washed with brine, dried over anhydrous Na₂SO₄, filtered and concentrated. The residue was purified by flash chromatography on silica gel (PE : EA = 50:1, V/V) to afford the desired product (600 mg, yield: 39%). LC/MS (ESI): m/z 228 [M+H]⁺.

### Step 2. 1-(2,6-dichloropyrimidin-4-yl)cyclohexan-1-ol (44-5)

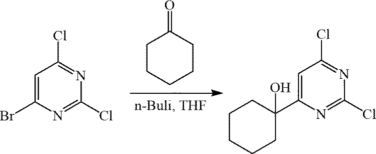

To a solution of 4-bromo-2,6-dichloropyrimidine (600 mg, 2.63 mmol) and cyclohexanone (0.32 mL, 3.16 mmol) in THF (15 mL) at -60° C. was added n-Butyllithium (2.5 M in THF, 1.5 mL, 3.69 mmol) dropwise. The mixture was stirred at -60° C. for 30 min. LC-MS showed the reaction was complete. The reaction mixture was quenched with saturated NH₄Cl aqueous solution and extracted with EA (60 mL). The organic layer was separated, then washed with brine, dried over anhydrous Na₂SO₄, filtered and concentrated. The residue was purified by flash chromatography on silica gel (PE : EA = 20:1, V/V) to afford the desired product (200 mg, yield: 30%). LC/MS (ESI): m/z 248 [M+H]⁺.

### Step 3. (R)-1-(2-chloro-6-(3-methylmorpholino)pyrimidin-4-yl)cyclohexan-1-ol (44-7)

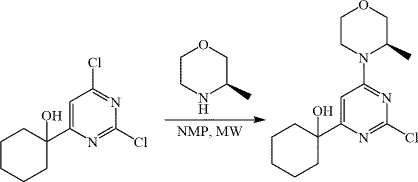

To a solution of 1-(2,6-dichloropyrimidin-4-yl)cyclohexan-1-ol (200 mg, 0.81 mmol) in NMP (5 mL) was added (R)-3-methylmorpholine (246 mg, 2.43 mmol). The mixture was stirred at 120° C. for 1 h under microwave irradiation. LC-MS showed the reaction was complete. The mixture was diluted with EA (60 mL), then washed with water and brine, dried over anhydrous Na₂SO₄, filtered and concentrated. The residue was purified by flash chromatography on silica gel (PE : EA = 10:1, V/V) to afford the desired product (200 mg, yield: 79%). LC/MS (ESI): m/z 312 [M+H]⁺.

### Step 4. (R)-1-(2-((1H-pyrazol-5-yl)amino)-6-(2-methylpiperidin-1-yl)pyrimidin-4-yl) cyclohexan-1-ol (44)

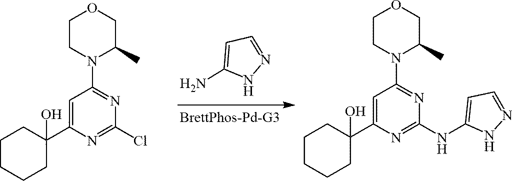

To a solution of (R)-1-(2-chloro-6-(3-methylmorpholino)pyrimidin-4-yl)cyclohexan-1-ol (80 mg, 0.26 mmol) in Dioxane (2 mL) were added 1H-pyrazol-5-amine (32 mg, 0.39 mmol), Cs₂CO₃ (167 mg, 0.51 mmol) and BrettPhos-Pd-G3 (23 mg, 0.03 mmol). The mixture was stirred at 80° C. overnight under nitrogen atmosphere. LC-MS showed the reaction was complete. The mixture was diluted with EA (50 mL), then washed with water and brine, dried over anhydrous Na₂SO₄, filtered and concentrated. The residue was purified by Prep -HPLC (C18, 10-95%, MeOH in H₂O with 0.1% HCOOH) to afford the desired product (20 mg, yield: 21%). LC/MS (ESI) m/z: 317 [M+H]⁺. ¹H NMR (400 MHz, DMSO) δ 8.99 (s, 1H), 7.50 (d, J= 2.0 Hz, 1H), 6.41 (d, J = 1.7 Hz, 1H), 6.38 (s, 1H), 4.86 (s, 1H), 4.35 (d, J = 4.7 Hz, 1H), 3.94 (dd, J= 19.7, 8.5 Hz, 2H), 3.73 (d, J= 11.3 Hz, 1H), 3.58 (d, J= 8.5 Hz, 1H), 3.43 (d, J= 2.9 Hz, 1H), 3.12 (dd, J= 12.6, 9.2 Hz, 1H), 1.85 (dt, J= 12.2, 7.2 Hz, 2H), 1.69 (dd, J = 24.2, 11.5 Hz, 3H), 1.49 (t, J = 13.8 Hz, 4H), 1.24 (s, 1H), 1.18 (d, J= 6.7 Hz, 3H).

### Example 39

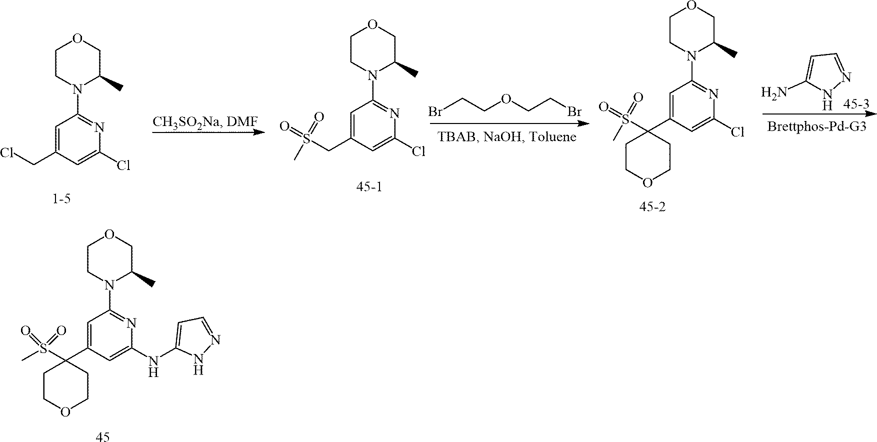

### Step 1. (R)-4-(6-chloro-4-((methylsulfonyl)methyl)pyridin-2-yl)-3-methylmorpholine (45-1)

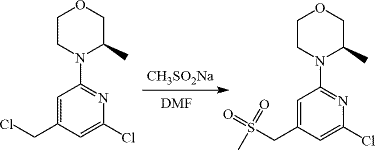

To a solution of (3R)-4-[6-chloro-4-(chloromethyl)pyridin-2-yl]-3-methylmorpholine (2 g, 7.66 mmol) in DMF (40 mL) was added CH₃SO₂Na (1.56 g, 15.32 mmol). The mixture was stirred at room temperature for 16 h. LC-MS showed the reaction was complete. The reaction mixture was diluted with EA (100 mL), then washed with water and brine, dried over anhydrous Na₂SO₄, filtered and concentrated to dryness. The residue was purified by column chromatography on silica gel (PE : EA = 10:1, V/V) to give the desired product (1.6 g, yield: 68%). LC/MS (ESI): m/z 305 [M+H]⁺.

### Step 2. (R)-4-(6-chloro-4-(4-(methylsulfonyl)tetrahydro-2H-pyran-4-yl)pyridin-2-yl)-3-methylmorpholine (45-2)

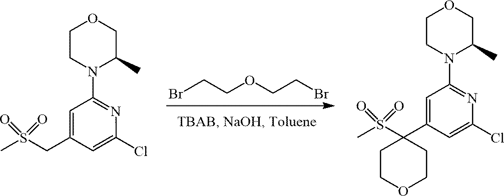

To a solution of (R)-4-(6-chloro-4-((methylsulfonyl)methyl)pyridin-2-yl)-3-methylmorpholine (800 mg, 2.62 mmol), 1-bromo-2-(2-bromoethoxy)ethane (1.83 g, 7.87 mmol) and TBAB (170 mg, 0.53 mmol) in Toluene (26 mL) was added NaOH aqueous solution (10 M, 2.63 mL, 26.25 mmol). The mixture was stirred at 60° C. for 16 h. LC-MS showed the reaction was complete. The reaction mixture was diluted with EA (60 mL), then washed with water and brine, dried over anhydrous Na₂SO₄, filtered and concentrated to dryness. The residue was purified by column chromatography on silica gel (PE : EA = 10:1, V/V) to give the desired product (550 mg, yield: 56%). LC/MS (ESI): m/z 375 [M+H]⁺.

### Step 3. (R)-6-(3-methylmorpholino)-4-(4-(methylsulfonyl)tetrahydro-2H-pyran-4-yl)-N-(1H-pyrazol-5-yl)pyridin-2-amine (45)

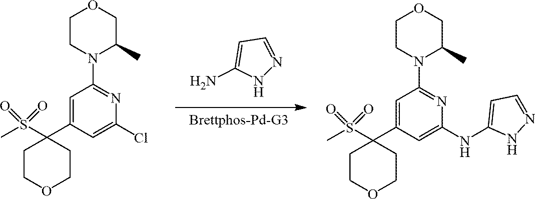

To a solution of (R)-4-(6-chloro-4-(4-(methylsulfonyl)tetrahydro-2H-pyran-4-yl) pyridin-2-yl)-3-methylmorpholine (100 mg, 0.27 mmol), 1H-pyrazol-5-amine (44 mg, 0.53 mmol) and Cs₂CO₃ (261 mg, 0.80 mmol) in dioxane (8 mL) was added BrettPhos -Pd-G3 (24 mg, 0.03 mmol). The mixture was stirred at 100° C. for 5 h. LC-MS showed the reaction was complete. The reaction mixture was diluted with EA (60 mL), then washed with water and brine, dried over anhydrous Na₂SO₄, filtered and concentrated to dryness. The residue was purified by Prep-HPLC (C₁₈, 10-95%, MeOH in H₂O with 0.1% HCOOH) to give the desired product (37 mg, yield: 33%). LC/MS (ESI): m/z 422 [M+H]⁺. ¹H NMR (400 MHz, DMSO) δ 11.94 (s, 1H), 8.98 (s, 1H), 7.54 (d, J = 2.2 Hz, 1H), 6.61 (s, 1H), 6.35 (d, J = 2.1 Hz, 1H), 6.18 (s, 1H), 4.32 - 4.23 (m, 1H), 3.97 - 3.83 (m, 4H), 3.73 (d, J = 11.1 Hz, 1H), 3.64 (dd, J = 11.2, 2.8 Hz, 1H), 3.52 - 3.46 (m, 1H), 3.26 - 3.20 (m, 2H), 3.06 (td, J = 12.7, 3.7 Hz, 1H), 2.71 (s, 3H), 2.44 (d, J = 13.7 Hz, 2H), 2.25 - 2.14 (m, 2H), 1.12 (d, J = 6.6 Hz, 3H).

### Example 40

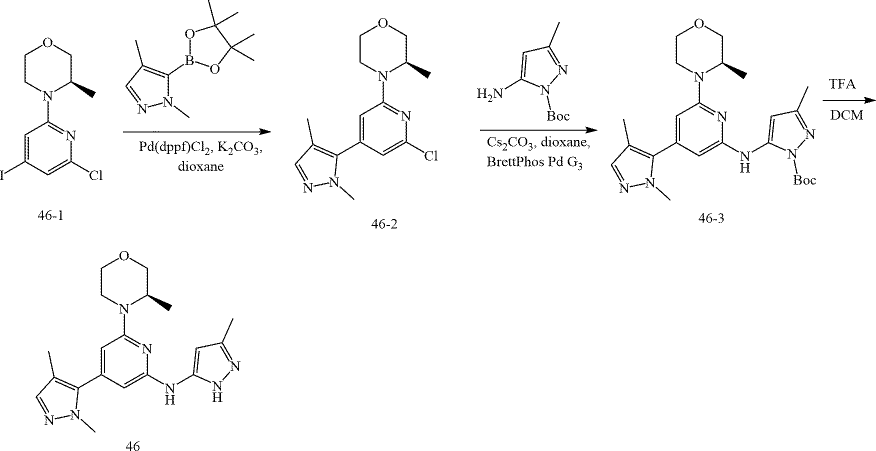

### Step 1: (R)-4-(6-chloro-4-(1,4-dimethyl-1H-pyrazol-5-yl)pyridin-2-yl)-3-methylmorpholine (46-2)

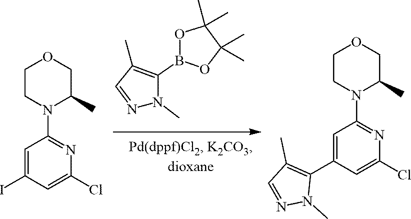

To a solution of (R)-4-(6-chloro-4-iodopyridin-2-yl)-3-methylmorpholine (150 mg, 0.44 mmol) and 1,4-dimethyl-5-(4,4,5,5-tetramethyl-1,3,2-dioxaborolan-2-yl)-1H-pyrazole (108.24 mg, 0.49 mmol) in dioxane (5 mL) were added K₂CO₃ (121.44 mg, 0.89 mmol) and Pd(dppf)Cl₂ (32.20 mg, 0.04 mmol). The mixture was charged with N₂ twice, then stirred at 90° C. for 12 hrs. The reaction mixture was diluted with water (10 mL) and extracted with EA (15 mL × 2). The combined organic layer was washed with brine (10 mL), dried over anhydrous Na₂SO₄, filtered and concentrated in vacuo. The resulting mixture was purified by flash chromatography eluting with PE/EtOAc (3:1, V/V) to afford the desired product (110 mg, yield: 80.93%).

### Step 2: Tert-butyl (R)-5-((4-(1,4-dimethyl-1H-pyrazol-5-yl)-6-(3-methylmorpholino)pyridin-2-yl)amino)-3-methyl-1H-pyrazole-1-carboxylate (46-3)

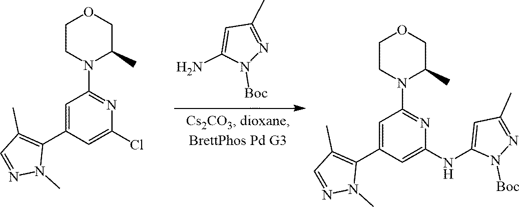

To a solution of (R)-4-(6-chloro-4-(1,4-dimethyl-1H-pyrazol-5-yl)pyridin-2-yl)-3-methylmorpholine (110 mg, 0.36 mmol) and tert-butyl 5-amino-3-methyl-1H-pyrazole-1-carboxylate (84.86 mg, 0.43 mmol) in dioxane (5 mL) were added CS₂CO₃ (350.47 mg, 1.08 mmol) and BrettPhos Pd G3 (32.67 mg, 0.036 mmol). The mixture was charged with N₂ twice, then stirred at 90° C. overnight. The reaction was diluted with water and extracted with EtOAc (10 mL×2). The combined organic layer was washed with brine (10 mL), dried over anhydrous Na₂SO₄, filtered and concentrated in vacuo. The resulting mixture was purified by flash chromatography eluting with PE/EtOAc (2:1, V/V) to afford the desired product (120 mg, yield: 71.58%).

### Step 3: (R)-4-(1,4-dimethyl-1H-pyrazol-5-yl)-N-(3-methyl-1H-pyrazol-5-yl)-6-(3-methylmorpholino)pyridin-2-amine (46)

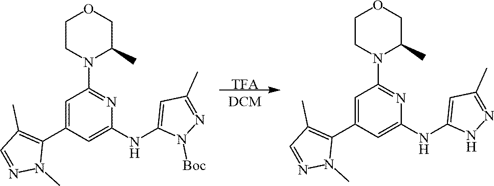

A mixture of tert-butyl (R)-5-((4-(1,4-dimethyl-1H-pyrazol-5-yl)-6-(3-methyl morpholino)pyridin-2-yl)amino)-1H-pyrazole-1-carboxylate (50 mg, 0.11 mmol) in DCM (3 mL) was added TFA (1 mL), the mixture was stirred at room temperature for 1 h. The resulting mixture was concentrated in vacuo. The saturated solution of NaHCO3 was added to the mixture until pH=7~8, and extracted with DCM (10 mL×2). The combined organic layer was washed with brine (10 mL), dried over anhydrous Na₂SO₄, filtered and concentrated in vacuo. The residue was purified by Pre-TLC (DCM/MeOH=10/1, V/V) to afford the desired product (10 mg, yield 25.45%). LC/MS (ESI) m/z: 368.1 [M+H]⁺. ¹H NMR (400 MHz, DMSO-d₆) δ ppm 11.69 (br s, 1 H) 8.88 (br s, 1 H) 7.31 (s, 1 H) 6.49 (br s, 1 H) 6.05 (br s, 1 H) 5.98 -6.01 (m, 1 H) 4.29 (br d, J=6.16 Hz, 1 H) 3.86 - 3.96 (m, 2 H) 3.73 (s, 3 H) 3.69 -3.72 (m, 1 H) 3.61 - 3.66 (m, 1 H) 3.45 - 3.52 (m, 1 H) 3.07 (td, J=12.58, 3.34 Hz, 1 H) 2.18 (s, 3 H) 1.98 (s, 3 H) 1.15 (d, J=6.54 Hz, 3 H).

### Example 41

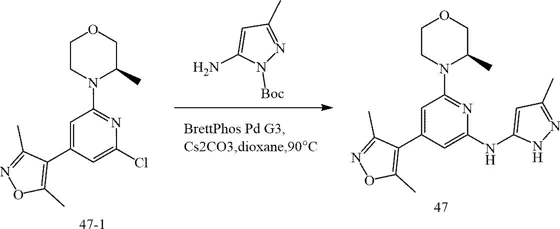

### Step 1. (R)-4-(3,5-dimethylisoxazol-4-yl)-N-(3-methyl-1H-pyrazol-5-yl)-6-(3-methylmorpholino)pyridin-2-amine (47)

To a (R)-4-(6-chloro-4-(3,5-dimethylisoxazol-4-yl)pyridin-2-yl)-3-methylmorpholine (150 mg, 0.49 mmol) and tert-butyl 5-amino-1H-pyrazole-1-carboxylate (116 mg, 0.59 mmol) in dioxane (8 mL) were added Cs₂CO₃ (400 mg, 1.23 mmol) and BrettPhos Pd G3 (45 mg, 0.049 mmol). The mixture was charged with N₂ twice, then stirred at 90° C. overnight. The reaction was diluted with water and extracted with EtOAc (30 mL×2). The combined organic layer was washed with brine (50 mL), dried over anhydrous Na₂SO₄, filtered and concentrated in vacuo. The residue was purified by Pre-TLC (DCM/MeOH=10/1) to afford the desired product (30 mg, yield 16.67%). LC/MS (ESI) m/z: 369.1 [M+H]⁺.

¹H NMR (400 MHz, DMSO-d6) δ ppm 11.68 (br s, 1 H) 8.80 (br s, 1 H) 6.52 (br s, 1 H) 6.05 (br s, 1 H) 5.98 (s, 1 H) 4.33 - 4.34 (m, 1 H) 4.29 (br d, J=6.52 Hz, 1 H) 3.93 (br dd, J=11.16, 3.01 Hz, 1 H) 3.82 - 3.88 (m, 1 H) 3.70 - 3.74 (m, 1 H) 3.61 -3.65 (m, 1 H) 3.48 (td, J=11.72, 2.82 Hz, 1 H) 3.06 (td, J=12.68, 3.64 Hz, 1 H) 2.43 (s, 3 H) 2.25 (s, 3 H) 2.18 (s, 3 H) 1.15 (d, J=6.64 Hz, 3 H).

### Example 42

### Biochemical Assays

### Assay 1: ATR Inhibition Assay

Detection of ATR kinase activity utilized the Mobility shift assay to measure the phosphorylation of the substrate protein FAM-RAD17 (GL, Cat. No. 514318, Lot. No. P19042-MJ524315). The assay was developed and conducted at Chempartner. All the test compounds were dissolved in 100% DMSO at concentration of 20 mM, then prepare compounds and conducted the assay as follows:

1) Transfer 80 µl20 mM compound to 40 µl of 100% DMSO in a 96-well plate.

2) Serially dilute the compound by transferring 20 µl to 60 µl of 100% DMSO in the next well and so forth for a total of 10 concentrations.

3) Add 100 µl of 100% DMSO to two empty wells for no compound control and no enzyme control in the same 96-well plate. Mark the plate as source plate.

4) Transfer 40 µl of compound from source plate to a new 384-well plate as the intermediate plate.

5) Transfer 60 nl compounds to assay plate by Echo.

6) Add ATR kinase (Eurofins, Cat. No. 14-953, Lot. No. D14JP007N) into Kinase base buffer (50 mM HEPES, pH 7.5; 0.0015% Brij-35; 0.01% Triton) to prepare 2x enzyme solution, then add 10 µl of 2x enzyme solution to each well of the 384-well assay plate, incubate at room temperature for 10 min.

7) Add FAM-RAD17 and ATP (Sigma, Cat. No. A7699-1G, CAS No. 987-65-5) in the kinase base buffer to prepare 2x peptide solution, then add 10 µl to the assay plate.

8) Incubate at 28° C. for specified period of time. Add 40 µl of stop buffer (100 mM HEPES, pH 7.5; 0.015% Brij-35; 0.2% Coating Reagent #3; 50 mM EDTA) to stop reaction.

9) Collect data on Caliper. Convert conversion values to inhibition values.

$$\text{Precent inhibition = (max-conversion)/(max-min)} ∗ \text{100}$$

wherein “max” stands for DMSO control; “min” stands for low control.

Fit the data in XLFit excel add-in version 5.4.0.8 to obtain IC50 values. Equation used is:

$$Y = \text{Bottom + (Top-Bottom)/(1+(IC50/X)\textasciicircum HillSlope}$$

wherein X means concentration in a format not transformed to logarithms.

The following Table 2 lists the IC50 values for exemplary compounds of Formula (I).

TABLE 2

| Compound No. | ATR IC₅₀ (nM) |
|---|---|
| 1 | 14.4 |
| 4 | 16.3 |
| 5 | 18.5 |
| 6 | 16 |
| 7 | 28 |
| 8 | 13.2 |
| 9 | 22 |
| 10 | 10 |
| 12 | 13 |
| 19 | 10.3 |
| 23 | 15.5 |
| 24 | 17.0 |
| 25 | 11.8 |
| 26 | 21.99 |
| 27 | 15.7 |
| 28 | 30.4 |
| 35 | 33.3 |
| 38 | 12.3 |
| 41 | 2.5 |
| 42 | 19.7 |
| 43 | 52.7 |
| 44 | 39.3 |

### Assay 2: Tumor Cell Anti-proliferation Assay (CTG Assay)

Human colorectal cancer cells HT-29 (HTB-38) and LoVo (CCL-229) were selected for the CTG assay, the two cell lines were originally obtained from the American Type Culture Collection (ATCC). Add FBS and appropriate additives into base medium to prepare complete medium, then briefly rinse the cell layer with 0.25% (w/v) Trypsin-0.038% (w/v) EDTA solution to remove all traces of serum that contains trypsin inhibitor, after that, add appropriate volume of Trypsin-EDTA solution to flask and observe cells under an inverted microscope until cell layer is dispersed, at last, add appropriate volume of complete growth medium and aspirate cells by gently pipetting. Collect and count numbers with Vi-cell XR and adjust cell density, seed cells into 96-well opaque-walled clear bottom tissue-culture treated plates in the CO2 incubator for 20-24 hours. All the test compounds will be at 10 mM in DMSO. Compounds are then added to the cell media in 3-fold serial dilutions, the final DMSO concentration is 0.5%. Incubate plates for 96 h at 5% CO₂, 37° C. Before the measurement, transfer the appropriate volume of CellTiter-Glo Buffer into the amber bottle containing CellTiter-Glo substrate to reconstitute the lyophilized enzyme/substrate mixture, mix gently, this forms the CellTiter-Glo Reagent (Promega Cat. No. G7573). Equilibrate the plate and its contents to room temperature for approximately 30 minutes, then add 100 µL of CellTiter-Glo Reagent to the assay plate, mix contents for 2 minutes on an orbital shaker to induce cell lysis, incubate at room temperature for 10 minutes to stabilize luminescent signal, at last paste the clear bottom with white back seal and record luminescence with Enspire. IC₅₀ and GI₅₀ values were calculated with XLFit curve fitting software using 4 Parameter Logistic Model Y=Bottom + (Top-Bottom)/(1+(IC50/X)^HillSlope).

The following Table 3 provides the IC50 (Y=50%) values for exemplary compounds of Formula (I).

TABLE 3

| Compound No. | LoVo IC50 (nM) |
|---|---|
| 1 | 334 |
| 4 | 385 |
| 5 | 653 |
| 6 | 401 |
| 7 | 872 |
| 8 | 397.8 |
| 9 | 538 |
| 10 | 262.9 |
| 11 | 1307 |
| 12 | 611 |
| 13 | 807 |
| 15 | 515 |
| 17 | 594 |
| 19 | 225 |
| 22 | 192 |
| 23 | 388 |
| 24 | 428 |
| 25 | 309 |
| 26 | 517 |
| 27 | 564 |
| 28 | 406 |
| 33 | 842 |
| 34 | 442 |
| 35 | 718 |
| 36 | 966 |
| 37 | 800 |
| 38 | 492 |
| 39 | 844 |
| 40 | 698 |
| 41 | 370 |
| 42 | 640 |
| 43 | 653 |
| 44 | 397 |
| 45 | 1012 |
| 46 | 410 |
| 47 | 390 |

The foregoing description is considered as illustrative only of the principles of the present disclosure. Further, since numerous modifications and changes will be readily apparent to those skilled in the art, it is not desired to limit the invention to the exact construction and process shown as described above. Accordingly, all suitable modifications and equivalents may be considered to fall within the scope of the invention as defined by the claims that follow.

1. A compound having Formula (I): 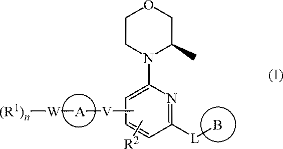
or a pharmaceutically acceptable salt thereof, wherein
Ring A is absent, 3- to 6-membered cycloalkyl, 5- to 6-membered heterocyclyl or 5- to 6-membered heteroaryl;
V is a direct bond, carbonyl or alkyl optionally substituted with one or more R^{c};
W and L are each independently a direct bond, —O—, —S—, or —N(R^{a})—;
R¹ is alkyl, cyano, hydroxyl, —S(O)₂CH₃, or —S(O)(NH)CH₃;
R² is hydrogen, halogen or alkyl optionally substituted with one or more R^{b};
Ring B is 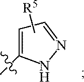
R⁵ is selected from the group consisting of hydrogen, halogen, hydroxyl, cyano, alkyl, alkenyl, alkynyl, heteroalkyl, heteroalkenyl, heteroalkynyl and haloalkyl;
R^{a} is hydrogen or alkyl;
R^{b} is hydroxyl or halogen;
R^{c} is hydroxyl, halogen or alkyl;
n is 0, 1, 2, or 3.

2-82. (canceled)

83. The compound of claim 1, or a pharmaceutically acceptable salt thereof, wherein Ring A is selected from the group consisting of absent, cyclopropyl, cyclopentyl, cyclohexyl, pyrazolyl, tetrahydropyranyl, thiazolyl, triazolyl, pyridyl and isoxazolyl.

84. The compound of claim 1, or a pharmaceutically acceptable salt thereof, wherein:
Ring A is absent, and W is —N(R^{a})—, wherein R^{a} is hydrogen or methyl, or
Ring A is absent, and W is a direct bond.

85. The compound of claim 1, or a pharmaceutically acceptable salt thereof, wherein
Ring A is 3- to 6-membered cycloalkyl, 5- to 6-membered heterocyclyl or 5- to 6-membered heteroaryl, preferably cyclopropyl, cyclohexyl, tetrahydropyranyl, thiazolyl, triazolyl, pyridyl or isoxazolyl, and R¹ is alkyl, hydroxyl, —S(O)₂CH₃ or —S(O)(NH)CH₃, or
Ring A is 5- to 6-membered heterocyclyl, preferably pyrazolyl, triazolyl or isoxazolyl, and R¹ is alkyl, or
Ring A is absent, and R¹ is cyano or —S(O)₂CH₃.

86. The compound of claim 1, or a pharmaceutically acceptable salt thereof, wherein L is —O—, —S— or -N(R^{a})-, Ring B is 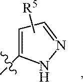
wherein R
^{a} is hydrogen, R⁵ is hydrogen or alkyl.

87. The compound of claim 1, or a pharmaceutically acceptable salt thereof, wherein R² is hydrogen, fluoro, or C₁₋₃ alkyl substituted with one or more R^{b}, wherein R^{b} is hydroxyl or fluoro.

88. The compound of claim 1, or a pharmaceutically acceptable salt thereof, having a formula selected from the group consisting of: 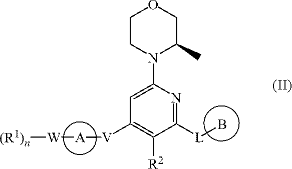 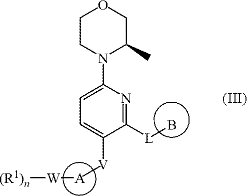 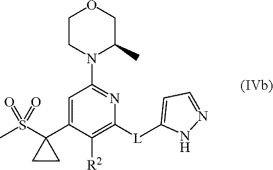 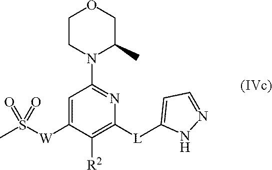 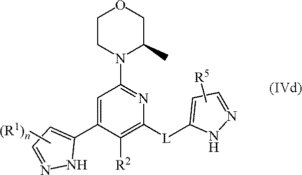 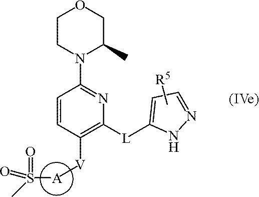 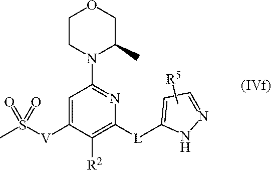 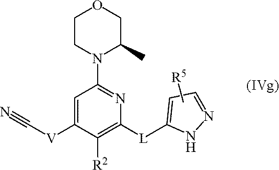
wherein
U is O or NH;
V is a direct bond, carbonyl or alkyl optionally substituted with one or more R^{c};
W and L are each independently —O—, —S— or —N(R^{a})—;
R¹ is alkyl;
R² is hydrogen, halogen, or alkyl substituted with one or more R^{b};
R⁵ is hydrogen or alkyl;
R^{a} is hydrogen or alkyl;
R^{b} is hydroxyl or halogen; and
R^{c} is hydroxyl, halogen or alkyl.

89. A compound having Formula (V): 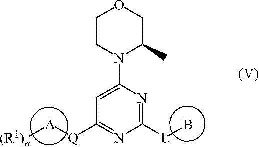
or a pharmaceutically acceptable salt thereof, wherein
Ring A is absent, 3- to 6-membered cycloalkyl, 5- to 6-membered heterocyclyl or 5- to 6-membered heteroaryl;
Q is a direct bond or alkyl optionally substituted with one or more R^{d};
L is —O—, —S— or —N(R^{a})—;
Ring B is 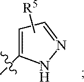
R^{a} is hydrogen or alkyl;
R^{d} is hydroxyl, halogen or alkyl;
R¹ is selected from the group consisting of cyano, hydroxyl, halogen, —S(O)₂CH₃, and -S(O)(NH)CH₃;
R⁵ is selected from the group consisting of hydrogen, halogen, hydroxyl, cyano, alkyl, alkenyl, alkynyl, heteroalkyl, heteroalkenyl, heteroalkynyl and haloalkyl;
n is 0, 1, 2, or 3.

90. The compound of claim 89, or a pharmaceutically acceptable salt thereof, wherein Ring A is absent, 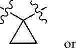 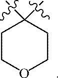
.

91. The compound of claim 89, or a pharmaceutically acceptable salt thereof, wherein
Q is alkyl and Ring A is absent, or
Q is a direct bond and Ring A is 3- to 6-membered cycloalkyl or 5- to 6-membered heterocyclyl.

92. The compound of claim 89, or a pharmaceutically acceptable salt thereof, wherein
Ring A is absent, 3- to 6-membered cycloalkyl or 5- to 6-membered heterocyclyl, and R¹ is —S(O)₂CH₃ or —S(O)(NH)CH₃, or
Ring A is absent or 3- to 6-membered cycloalkyl, and R¹ is cyano, hydroxyl or halogen.

93. The compound of claim 89, or a pharmaceutically acceptable salt thereof, wherein Ring B is 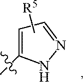
wherein R
⁵ is hydrogen or alkyl.

94. A compound selected from the group consisting of: 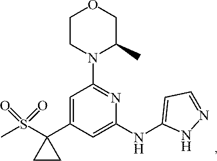 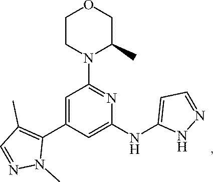 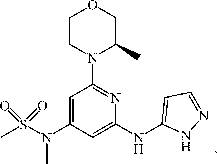 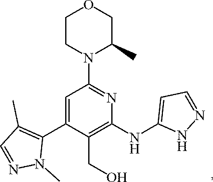 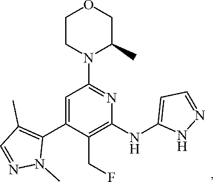 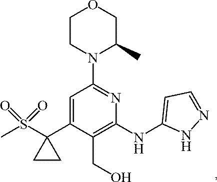 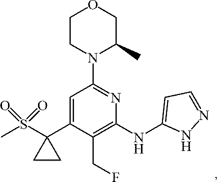 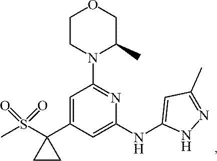 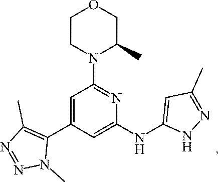 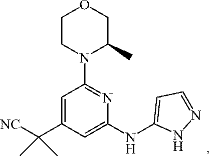 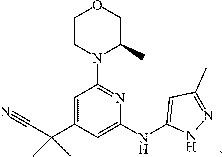 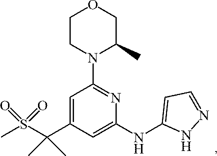 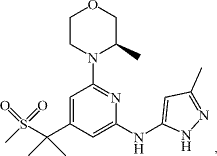 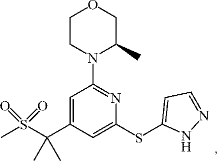 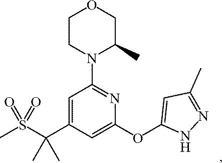 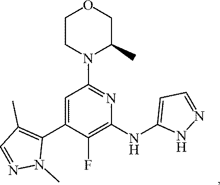 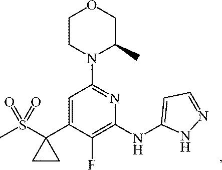 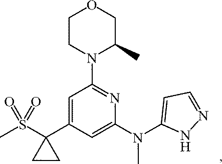 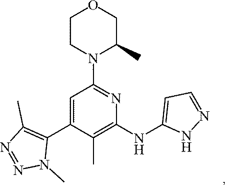 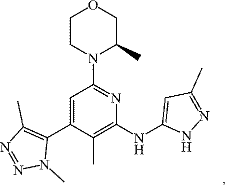 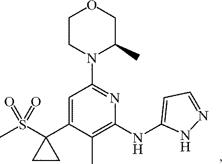 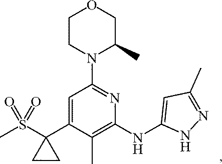 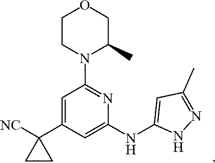 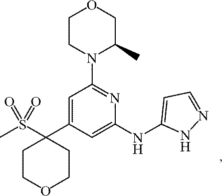 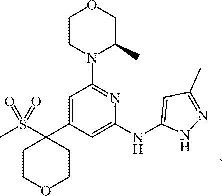 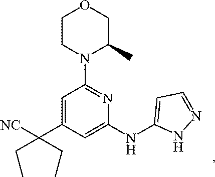 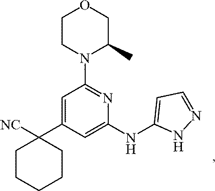 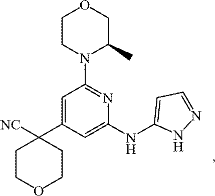 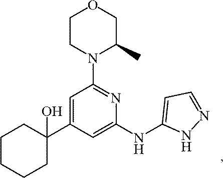 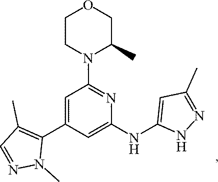 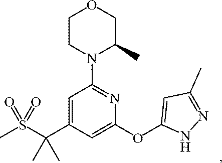 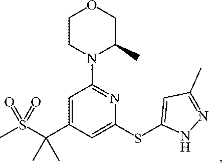 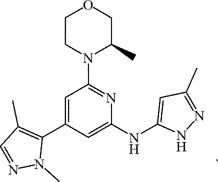 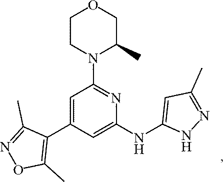 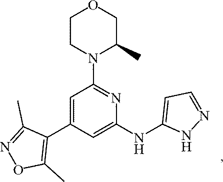 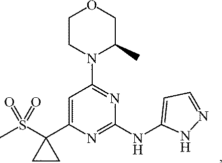 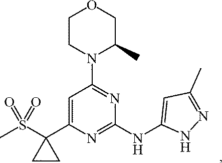 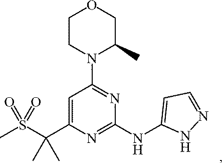 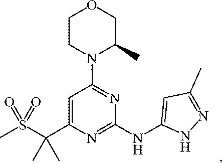 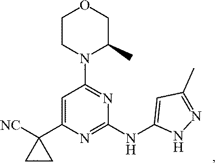 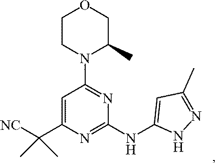 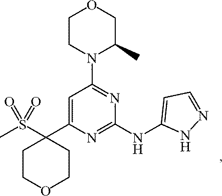 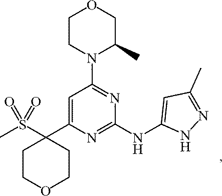 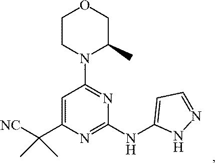 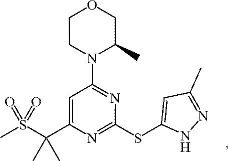 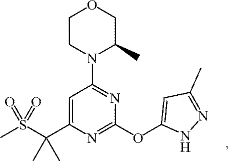 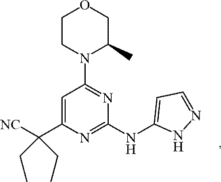 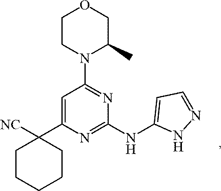 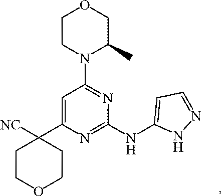 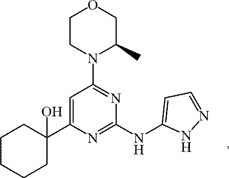 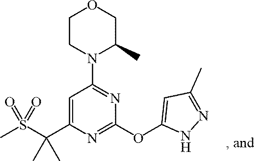 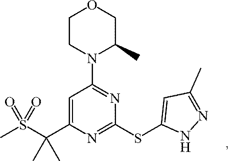
or a pharmaceutically acceptable salt thereof.

95. A pharmaceutical composition comprising the compound of claim 1 or a pharmaceutically acceptable salt thereof, and a pharmaceutically acceptable carrier.

96. A pharmaceutical composition comprising the compound of claim 89 or a pharmaceutically acceptable salt thereof, and a pharmaceutically acceptable carrier.

97. A pharmaceutical composition comprising the compound of claim 94 or a pharmaceutically acceptable salt thereof, and a pharmaceutically acceptable carrier.

98. A method for treating cancer, comprising administering an effective amount of a compound of claim 1 or a pharmaceutically acceptable salt thereof to a subject in need thereof.

99. A method for treating cancer, comprising administering an effective amount of a compound of claim 89 or a pharmaceutically acceptable salt thereof to a subject in need thereof.

100. A method for treating cancer, comprising administering an effective amount of a compound of claim 94 or a pharmaceutically acceptable salt thereof to a subject in need thereof.

101. A method for inhibiting ATR kinase in a subject in need thereof, comprising administering an effective amount of a compound of claim 1 to the subject.

102. A method for inhibiting ATR kinase in a subject in need thereof, comprising administering an effective amount of a compound of claim 89 to the subject.

103. A method for inhibiting ATR kinase in a subject in need thereof, comprising administering an effective amount of a compound of claim 94 to the subject.
